(19) United States
(12) Patent Application Publication
(10) Pub. No.: US 2022/0184070 A1
(43) Pub. Date: Jun. 16, 2022

(54) LOW-DOSE TRIPLE COMBINATION FORMULATION

(71) Applicant: The George Institute for Global Health, Newtown, Sydney, NSW (AU)

(72) Inventors: Anthony RODGERS, Newtown, Sydney, NSW (AU); Stephen MACMAHON, Newtown, Sydney, NSW (AU)

(21) Appl. No.: 17/598,122

(22) PCT Filed: Mar. 25, 2020
(86) PCT No.: PCT/IB2020/000141

§ 371 Date: Sep. 24, 2021

Related U.S. Application Data
(60) Provisional application No. 62/823,575, filed on Mar. 25, 2019.

(51) Int. Cl. A61K 31/4985; A61K 31/155; A61K 31/64; A61P 3/10

(52) U.S. Cl. CPC A61K 31/4985; A61P 3/10; A61K 31/64; A61K 31/155

(57) ABSTRACT

Pharmaceutical compositions useful in the treatment of metabolic disorders including diabetes, the compositions comprising a) a dipeptidyl peptidase IV (DPP IV) inhibitor such as sitagliptin, b) a biguanide such as metformin, and c) a sulfonylurea such as glimepiride, wherein each one of the DPPIV inhibitor, biguanide and sulfonylurea are at a dose that is at about 20-75% of the lowest diabetes therapeutic dose (LDTD).

### CROSS-REFERENCE

This application claims the benefit of U.S. Provisional Patent Application No. 62/823,575 filed on Mar. 25, 2019, which is incorporated by reference in its entirety.

### BACKGROUND

Diabetes mellitus (DM), commonly referred to as diabetes, is a group of metabolic disorders in which there are high blood sugar levels over a prolonged period. Symptoms of high blood sugar include frequent urination, increased thirst, and increased hunger. If left untreated, diabetes can cause many complications.

### SUMMARY OF THE DISCLOSURE

Provided herein, in certain embodiments, are pharmaceutical compositions comprising: a) a dipeptidyl peptidase IV (DPP IV) inhibitor; b) a biguanide; c) a sulfonylurea; and d) at least one pharmaceutically-acceptable excipient wherein (a), (b), and (c) are each at about 20% to about 75% of the lowest diabetes therapeutic dose (LDTD). Provided herein, in certain embodiments, are pharmaceutical compositions consisting essentially of: a) a DPP IV inhibitor; b) a biguanide; c) a sulfonylurea; and d) at least one pharmaceutically-acceptable excipient; wherein (a), (b), and (c) are each at about 20% to about 75% of the lowest diabetes therapeutic dose (LDTD). Provided herein, in certain embodiments, are pharmaceutical compositions, comprising: a) a dipeptidyl peptidase IV (DPP IV) inhibitor; b) a biguanide; c) a sulfonylurea; and d) at least one pharmaceutically-acceptable excipient wherein (a), (b), and (c) are each at about 65%-75% of the lowest diabetes therapeutic dose (LDTD) for each of the (a) and (b), and (c) is at about 45%-55% of the lowest diabetes therapeutic dose (LDTD) for (c). Provided herein, in certain embodiments, are pharmaceutical compositions consisting essentially of: a) a DPP IV inhibitor; b) a biguanide; c) a sulfonylurea; and d) at least one pharmaceutically-acceptable excipient; wherein (a), (b), and (c) are each at about 65%-75% of the lowest diabetes therapeutic dose (LDTD) for each of the (a) and (b), and (c) is at about 45%-55% of the lowest diabetes therapeutic dose (LDTD) for (c). In some embodiments, the DPP IV inhibitor is a gliptin. In some embodiments, the DPP IV inhibitor is selected from sitagliptin, vildagliptin, saxagliptin, linagliptin, gemigliptin, anagliptin, teneligliptin, alogliptin, trelagliptin, omarigliptin, evogliptin, gosogliptin, dutogliptin, or the pharmaceutically acceptable salt or hydrate thereof. In some embodiments, the DPP IV inhibitor is sitagliptin or the pharmaceutically acceptable salt thereof. In some embodiments, the DPP IV inhibitor is sitagliptin phosphate. In some embodiments, the biguanide is metformin or the pharmaceutically acceptable salt or hydrate thereof. In some embodiments, the biguanide is metformin hydrochloride. In some embodiments, the metformin is formulated for immediate release. In some embodiments, the metformin is formulated for slow release. In some embodiments, the sulfonylurea is selected from acetohexamide, carbutamide, chlorpropamide, glycyclamide (tolhexamide), metahexamide, tolazamide, tolbutamide, glibenclamide (glyburide), glibornuride, gliclazide, glipizide, gliquidone, glisoxepide, glyclopyramide, glimepiride, or the pharmaceutically acceptable salt or hydrate thereof. In some embodiments, the sulfonylurea is glimepiride. In some embodiments, the dose of each (a), (b), and (c) is from about 40% to about 75% of the lowest diabetes therapeutic dose (LDTD). In some embodiments, the dose of each (a), (b), and (c) is from about 60% to about 75% of the lowest diabetes therapeutic dose (LDTD). In some embodiments, the dose of each (a), (b), and (c) is from about 65% to about 75% of the lowest diabetes therapeutic dose (LDTD). In some embodiments, the dose of each (a), (b), and (c) is about 70% of the lowest diabetes therapeutic dose (LDTD). In some embodiments, the dose of each (a), (b), and (c) is from about 40% to about 70% of the lowest diabetes therapeutic dose (LDTD). In some embodiments, the dose of each (a), (b), and (c) is from about 40% to about 60% of the lowest diabetes therapeutic dose (LDTD). In some embodiments, the dose of each (a), (b), and (c) is from about 45% to about 55% of the lowest diabetes therapeutic dose (LDTD). In some embodiments, the dose of each (a), (b), and (c) is about 50% of the lowest diabetes therapeutic dose (LDTD). In some embodiments, the DPP IV inhibitor is about 50% of the lowest diabetes therapeutic dose (LDTD) for the DPP IV inhibitor. In some embodiments, the DPP IV inhibitor is sitagliptin, and the dose of sitagliptin is about 12.5 mg. In some embodiments, the DPP IV inhibitor is about 70% of the lowest diabetes therapeutic dose (LDTD) for the DPP IV inhibitor. In some embodiments, the DPP IV inhibitor is sitagliptin, and the dose of sitagliptin is about 17.5 mg. In some embodiments, the biguanide is about 50% of the lowest diabetes therapeutic dose (LDTD) for the biguanide. In some embodiments, the biguanide is metformin hydrochloride, and the dose of metformin hydrochloride is about 250 mg. In some embodiments, the biguanide is about 70% of the lowest diabetes therapeutic dose (LDTD) for the biguanide. In some embodiments, the biguanide is metformin hydrochloride, and the dose of metformin hydrochloride is about 350 mg. In some embodiments, the sulfonylurea is about 70% of the lowest diabetes therapeutic dose (LDTD) for the sulfonylurea. In some embodiments, the sulfonylurea is glimepiride, and the dose of sulfonylurea is about 0.7 mg. In some embodiments, the sulfonylurea is about 50% of the lowest diabetes therapeutic dose (LDTD) for the sulfonylurea. In some embodiments, the sulfonylurea is glimepiride, and the dose of sulfonylurea is about 0.5 mg. In some embodiments, the DPP IV inhibitor is sitagliptin, the biguanide is metformin, and the sulfonylurea is glimepiride. In some embodiments, the dose of sitagliptin is from about 5.0 mg to about 18.75 mg, the dose of metformin is from about 100 mg to about 375 mg, and the dose of glimepiride is from about 0.2 mg to about 0.75 mg. In some embodiments, the dose of sitagliptin is from about 10 mg to about 16.25 mg, the dose of metformin is from about 200 mg to about 325 mg, and the dose of glimepiride is from about 0.4 mg to about 0.65 mg. In some embodiments, the dose of sitagliptin is from about 10 mg to about 15 mg, the dose of metformin is from about 200 mg to about 300 mg, and the dose of glimepiride is from about 0.4 mg to about 0.6 mg. In some embodiments, the dose of sitagliptin is from about 11.25 mg to about 13.75 mg, the dose of metformin is from about 225 mg to about 275 mg, and the dose of glimepiride is from about 0.45 mg to about 0.55 mg. In some embodiments, the dose of sitagliptin is about 12.5 mg, the dose of metformin is about 250 mg, and the dose of glimepiride is about 0.5 mg. In some embodiments, the dose of sitagliptin is about 17.5 mg, the dose of metformin is about 350 mg, and the dose of glimepiride is about 0.5 mg. In some embodiments, the dose of each (a), (b), and (c) is from about 30% to about 40% of the lowest diabetes therapeutic dose (LDTD). In some embodiments, the dose of each (a), (b), and (c) is from about 30% to about 35% of the lowest diabetes therapeutic dose (LDTD). In some embodiments, the sulfonylurea is about 33% of the lowest diabetes therapeutic dose (LDTD) for the sulfonylurea. In some embodiments, the sulfonylurea is glimepiride, and the dose of sulfonylurea is about 0.33 mg. In some embodiments, the dose of sitagliptin is about 8.25 mg, the dose of metformin is about 165 mg, and the dose of glimepiride is about 0.33 mg. In some embodiments, the dose of sitagliptin is from about 7.5 mg to about 10 mg, the dose of metformin is from about 150 mg to about 200 mg, and the dose of glimepiride is from about 0.3 mg to about 0.4 mg. In some embodiments, the dose of each (a), (b), and (c) is from about 20% to about 30% of the lowest diabetes therapeutic dose (LDTD). In some embodiments, the dose of each (a), (b), and (c) is from about 22% to about 28% of the lowest diabetes therapeutic dose (LDTD). In some embodiments, the sulfonylurea is about 25% of the lowest diabetes therapeutic dose (LDTD) for the sulfonylurea. In some embodiments, the sulfonylurea is glimepiride, and the dose of sulfonylurea is about 0.25 mg. In some embodiments, the DPP IV inhibitor is about 25% of the lowest diabetes therapeutic dose (LDTD) for the DPP IV inhibitor. In some embodiments, the DPP IV inhibitor is sitagliptin, and the dose of sitagliptin is about 6.25 mg and the dose of metformin is about 150 mg. In some embodiments, the biguanide is about 25% of the lowest diabetes therapeutic dose (LDTD) for the biguanide. In some embodiments, the biguanide is metformin hydrochloride, and the dose of metformin hydrochloride is about 125 mg. In some embodiments, the pharmaceutical composition comprises: (a) sitagliptin as a DPP IV; (b) metformin as a biguanide; and (c) glimepiride as a sulfonylurea. In some embodiments, the dose of sitagliptin is from about 5 mg to about 7.5 mg, the dose of metformin is from about 100 mg to about 150 mg, and the dose of glimepiride is from about 0.2 mg to about 0.3 mg. In some embodiments, the dose of sitagliptin is about 6.25 mg, the dose of metformin is about 150 mg, and the dose of glimepiride is about 0.25 mg. In some embodiments, (a), (b), and (c) are provided in one formulation. In some embodiments, (a), (b), and (c) are each provided in a separate formulation. In some embodiments, two of the (a), (b), and (c) are provided in one formulation. In some embodiments, the pharmaceutical composition is in the form of pill, tablet or capsule. In some embodiments, the pharmaceutical composition is suitable for oral administration.

Provided herein, in certain embodiments, are methods of treating diabetes in a subject in need thereof, comprising administering any one of the pharmaceutical compositions disclosed herein. In some embodiments, the treatment results in an improvement, slowing the progression of, or delaying a metabolic disorder such as diabetes mellitus, impaired glucose tolerance, impaired fasting blood glucose, hyperglycemia, postprandial hyperglycemia, overweight, obesity, metabolic syndrome, impaired renal function, gestational diabetes, new onset diabetes after transplantation (NODAT) and complications associated therewith, and post-transplant metabolic syndrome (PTMS) and complications associated therewith. In some embodiments, the treatment results in an improvement, slowing the progression of, or delaying a metabolic disorder that is greater than that obtained with the full lowest diabetes therapeutic dose (LDTD) dose of any one of (a), (b), and (c) in the pharmaceutical composition. In some embodiments, the treatment results in greater long term tolerability and reduced risk of side effects when compared to treatment with the lowest diabetes therapeutic dose (LDTD) of any one of (a), (b), and (c) in the pharmaceutical composition. In some embodiments, the treatment is the initial or first-line treatment of diabetes. In some embodiments, the subject is not receiving any diabetes therapy prior to treatment. In some embodiments, the subject is receiving diabetes therapy prior to treatment and treatment with the formulations disclosed herein is second-line or maintenance treatment.

Provided herein, in certain embodiments, are pharmaceutical compositions, comprising: a) a low-dose, therapeutically-effective amount of a dipeptidyl peptidase IV (DPP IV) inhibitor; b) a low-dose, therapeutically-effective amount of a biguanide; c) a low-dose, therapeutically-effective amount of a sulfonylurea; and d) a pharmaceutically acceptable excipient, wherein (a), (b), and (c) are each at about 20% to about 75% of a lowest diabetes therapeutic dose (LDTD). In some embodiments, the DPP IV inhibitor is a gliptin. In some embodiments, the DPP IV inhibitor is sitagliptin, vildagliptin, saxagliptin, linagliptin, gemigliptin, anagliptin, teneligliptin, alogliptin, trelagliptin, omarigliptin, evogliptin, gosogliptin, dutogliptin, or a pharmaceutically acceptable salt or hydrate thereof. In some embodiments, the DPP IV inhibitor is sitagliptin or a pharmaceutically acceptable salt thereof. In some embodiments, the DPP IV inhibitor is sitagliptin phosphate. In some embodiments, the biguanide is metformin or a pharmaceutically acceptable salt or hydrate thereof. In some embodiments, the biguanide is metformin hydrochloride. In some embodiments, the metformin is formulated for immediate release. In some embodiments, the metformin is formulated for slow release. In some embodiments, the sulfonylurea is acetohexamide, carbutamide, chlorpropamide, glycyclamide (tolhexamide), metahexamide, tolazamide, tolbutamide, glibenclamide (glyburide), glibornuride, gliclazide, glipizide, gliquidone, glisoxepide, glyclopyramide, glimepiride, or a pharmaceutically acceptable salt or hydrate thereof. In some embodiments, the sulfonylurea is glimepiride. In some embodiments, a dose of each (a), (b), and (c) is from about 40% to about 70% of the lowest diabetes therapeutic dose (LDTD). In some embodiments, a dose of each (a), (b), and (c) is from about 40% to about 60% of the lowest diabetes therapeutic dose (LDTD). In some embodiments, a dose of each (a), (b), and (c) is from about 45% to about 55% of the lowest diabetes therapeutic dose (LDTD). In some embodiments, the DPP IV inhibitor is about 70% of the lowest diabetes therapeutic dose (LDTD) for the DPP IV inhibitor. In some embodiments, the DPP IV inhibitor is sitagliptin, and a dose of sitagliptin is about 17.5 mg. In some embodiments, the DPP IV inhibitor is about 50% of the lowest diabetes therapeutic dose (LDTD) for the DPP IV inhibitor. In some embodiments, the DPP IV inhibitor is sitagliptin, and a dose of sitagliptin is about 12.5 mg. In some embodiments, the biguanide is about 70% of the lowest diabetes therapeutic dose (LDTD) for the biguanide. In some embodiments, the biguanide is metformin hydrochloride, and a dose of metformin hydrochloride is about 350 mg. In some embodiments, the biguanide is about 50% of the lowest diabetes therapeutic dose (LDTD) for the biguanide. In some embodiments, the biguanide is metformin hydrochloride, and a dose of metformin hydrochloride is about 250 mg. In some embodiments, the sulfonylurea is about 50% of the lowest diabetes therapeutic dose (LDTD) for the sulfonylurea. In some embodiments, the sulfonylurea is glimepiride, and a dose of the glimepiride is about 0.5 mg. In some embodiments, the DPP IV inhibitor is sitagliptin, the biguanide is metformin, and the sulfonylurea is glimepiride. In some embodiments, a dose of sitagliptin is from about 5.0 mg to about 18.75 mg, a dose of metformin is from about 100 mg to about 375 mg, and a dose of glimepiride is from about 0.2 mg to about 0.75 mg. In some embodiments, the dose of sitagliptin is from about 10 mg to about 16.25 mg, the dose of metformin is from about 200 mg to about 325 mg, and the dose of glimepiride is from about 0.4 mg to about 0.65 mg. In some embodiments, the dose of sitagliptin is from about 10 mg to about 15 mg, the dose of metformin is from about 200 mg to about 300 mg, and the dose of glimepiride is from about 0.4 mg to about 0.6 mg. In some embodiments, the dose of sitagliptin is from about 11.25 mg to about 13.75 mg, the dose of metformin is from about 225 mg to about 275 mg, and the dose of glimepiride is from about 0.45 mg to about 0.55 mg. In some embodiments, the dose of sitagliptin is about 12.5 mg, the dose of metformin is about 250 mg, and the dose of glimepiride is about 0.5 mg. In some embodiments, the dose of each (a), (b), and (c) is from about 30% to about 40% of the lowest diabetes therapeutic dose (LDTD). In some embodiments, the dose of each (a), (b), and (c) is from about 30% to about 35% of the lowest diabetes therapeutic dose (LDTD). In some embodiments, the sulfonylurea is about 33% of the lowest diabetes therapeutic dose (LDTD) for the sulfonylurea. In some embodiments, the sulfonylurea is glimepiride, and the dose of sulfonylurea is about 0.33 mg. In some embodiments, the DPP IV inhibitor is sitagliptin, the biguanide is metformin, and the sulfonylurea is glimepiride. In some embodiments, the dose of sitagliptin is from about 7.5 mg to about 10 mg, the dose of metformin is from about 150 mg to about 200 mg, and the dose of glimepiride is from about 0.3 mg to about 0.4 mg. In some embodiments, the dose of sitagliptin is about 8.25 mg, the dose of metformin is about 165 mg, and the dose of glimepiride is about 0.33 mg. In some embodiments, the dose of each (a), (b), and (c) is from about 20% to about 30% of the lowest diabetes therapeutic dose (LDTD). In some embodiments, the dose of each (a), (b), and (c) is from about 22% to about 28% of the lowest diabetes therapeutic dose (LDTD). In some embodiments, the sulfonylurea is about 25% of the lowest diabetes therapeutic dose (LDTD) for the sulfonylurea. In some embodiments, the sulfonylurea is glimepiride, and the dose of sulfonylurea is about 0.25 mg. In some embodiments, the DPP IV inhibitor is sitagliptin, the biguanide is metformin, and the sulfonylurea is glimepiride. In some embodiments, the dose of sitagliptin is from about 5mg to about 7.5 mg, the dose of metformin is from about 100 mg to about 150 mg, and the dose of glimepiride is from about 0.2 mg to about 0.3 mg. In some embodiments, the dose of sitagliptin is about 6.25 mg, the dose of metformin is about 150 mg, and the dose of glimepiride is about 0.25 mg. In some embodiments, the pharmaceutical composition is in the form of pill, tablet, or capsule. In some embodiments, the pharmaceutical composition is suitable for oral administration. In some embodiments, the pharmaceutical composition does not comprise any further additional anti-hyperglycemic or anti-diabetic agents. In some embodiments, the combination of a), b), and c) produces a synergistic effect. In some embodiments, the pharmaceutical composition produces a larger decrease in 2 hour post-prandial glucose as compared to a maximum decrease in the post-prandial glucose obtained from about 100 mg of sitagliptin. In some embodiments, the pharmaceutical composition produces a larger decrease in 2 hour post-prandial glucose as compared to a maximum decrease in the post-prandial glucose obtained from about 850 mg of metformin. In some embodiments, the pharmaceutical composition produces a larger decrease in 2 hour post-prandial glucose as compared to a maximum decrease in the post-prandial glucose obtained from about 1700 mg of metformin.

Provided herein, in certain embodiments, are pharmaceutical compositions, comprising: a) a low-dose, therapeutically-effective amount of a dipeptidyl peptidase IV (DPP IV) inhibitor; b) a low-dose, therapeutically-effective amount of a biguanide; c) a low-dose, therapeutically-effective amount of a sulfonylurea; and d) a pharmaceutically acceptable excipient, wherein (a) and (b) are each at about 65%-75% of a lowest diabetes therapeutic dose (LDTD), and (c) is at about 45%-55% of the lowest diabetes therapeutic dose (LDTD). In some embodiments, the DPP IV inhibitor is sitagliptin and a dose of sitagliptin is from about 16.25 mg to about 18.75 mg. In some embodiments, the biguanide is metformin and a dose of metformin is from about 325 mg to about 375 mg. In some embodiments, the sulfonylurea is glimepiride, and a dose of glimepiride from about 0.45 mg to about 0.55 mg. In some embodiments, the DPP IV inhibitor is at about 70% of the lowest diabetes therapeutic dose (LDTD) for the DPP IV inhibitor. In some embodiments, the biguanide is at about 70% of the lowest diabetes therapeutic dose (LDTD) for the biguanide. In some embodiments, the sulfonylurea is at about 50% of the lowest diabetes therapeutic dose (LDTD) for the sulfonylurea. In some embodiments, the DPP IV inhibitor is sitagliptin, the biguanide is metformin, and the sulfonylurea is glimepiride. In some embodiments, the dose of sitagliptin is about 17.5 mg, the dose of metformin is about 350 mg, and the dose of glimepiride is about 0.5 mg. In some embodiments, the DPP IV inhibitor is sitagliptin and the dose of the sitagliptin is about 17.5 mg. In some embodiments, the biguanide is metformin and the dose of the metformin is about 350 mg. In some embodiments, the sulfonylurea is glimepiride and the dose of the glimepiride is about 0.5 mg. In some embodiments, the pharmaceutical composition is suitable for oral administration. In some embodiments, the pharmaceutical composition is in the form of pill, tablet or capsule. In some embodiments, the metformin is formulated for immediate release. In some embodiments, the metformin is formulated for slow release. In some embodiments, the pharmaceutical composition does not comprise any further additional anti-hyperglycemic or anti-diabetic agents. In some embodiments, the combination of a), b), and c) produces a synergistic effect. In some embodiments, the pharmaceutical composition produces a larger decrease in 2 hour post-prandial glucose as compared to a maximum decrease in the post-prandial glucose obtained from about 100 mg of sitagliptin. In some embodiments, the pharmaceutical composition produces a larger decrease in 2 hour post-prandial glucose as compared to a maximum decrease in the post-prandial glucose obtained from about 850 mg of metformin. In some embodiments, the pharmaceutical composition produces a larger decrease in 2 hour post-prandial glucose as compared to a maximum decrease in the post-prandial glucose obtained from about 1700 mg of metformin.

Provided herein, in certain embodiments, are pharmaceutical compositions, comprising a combination of: a) about 17.5 mg of sitagliptin; b) about 350 mg of metformin; c) about 0.5 mg of glimepiride; and d) at least one pharmaceutically-acceptable excipient. In some embodiments, the combination is synergistic. In some embodiments, the pharmaceutical composition is in the form of pill, tablet, or capsule. In some embodiments, the pharmaceutical composition is suitable for oral administration. In some embodiments, the metformin is formulated for immediate release. In some embodiments, the metformin is formulated for slow release. In some embodiments, the pharmaceutical composition does not comprise any further additional anti-hyperglycemic or anti-diabetic agents. In some embodiments, the combination of a), b), and c) produces a synergistic effect. In some embodiments, the pharmaceutical composition produces a larger decrease in 2 hour post-prandial glucose as compared to a maximum decrease in the post-prandial glucose obtained from about 100 mg of sitagliptin. In some embodiments, the pharmaceutical composition produces a larger decrease in 2 hour post-prandial glucose as compared to a maximum decrease in the post-prandial glucose obtained from about 850 mg of metformin. In some embodiments, the pharmaceutical composition produces a larger decrease in 2 hour post-prandial glucose as compared to a maximum decrease in the post-prandial glucose obtained from about 1700 mg of metformin.

Provided herein, in certain embodiments, are synergistic, ultra-low dose, anti-diabetic drug combinations, consisting of: a) about 16.25 mg to about 18.75 mg of sitagliptin, or a salt or hydrate thereof; b) about 325 mg to about 375 mg of metformin, or a salt or hydrate thereof; c) about 0.45 mg to about 0.55 mg of glimepiride, or a salt or hydrate thereof; and d) at least one excipient. In some embodiments, the combination does not comprise any further additional anti-hyperglycemic or anti-diabetic agents. In some embodiments, the combination of a), b), and c) produces a synergistic effect. In some embodiments, the combination produces a larger decrease in 2 hour post-prandial glucose as compared to a maximum decrease in the post-prandial glucose obtained from about 100 mg of sitagliptin, from about 850 mg of metformin, or from about 1700 mg of metformin.

Provided herein, in certain embodiments, are methods of treating diabetes in a subject in need thereof comprising administering the pharmaceutical composition as described herein. In some embodiments, the subject has persisting elevation of blood sugar after treatment with one or two of a DPP IV inhibitor, a biguanide, or a sulfonylurea at the LDTD or higher dose. In some embodiments, the administration of the pharmaceutical composition is an initial or first-line treatment of diabetes.

Provided herein, in certain embodiments, are methods of improving, slowing the progression of, or delaying a metabolic disorder, wherein the metabolic disorder comprises diabetes mellitus, impaired glucose tolerance, impaired fasting blood glucose, hyperglycemia, postprandial hyperglycemia, overweight, obesity, metabolic syndrome, impaired renal function, gestational diabetes, new onset diabetes after transplantation (NODAT) and complications associated therewith, or post-transplant metabolic syndrome (PTMS) and complications associated therewith, comprising administering to a subject in need thereof the pharmaceutical composition as described herein.

Provided herein, in certain embodiments, are methods of treating diabetes in a subject in need thereof comprising administering a synergistic, ultra-low dose, anti-diabetic drug combination, consisting of: a) about 16.25 mg to about 18.75 mg of sitagliptin, or a salt or hydrate thereof; b) about 325 mg to about 375 mg of metformin, or a salt or hydrate thereof; c) about 0.45 mg to about 0.55 mg of glimepiride, or a salt or hydrate thereof; and d) at least one excipient. In some embodiments, the combination does not comprise any further additional anti-hyperglycemic or anti-diabetic agents. In some embodiments, the combination of a), b), and c) produces a synergistic effect. In some embodiments, the combination produces a larger decrease in 2 hour post-prandial glucose as compared to a maximum decrease in the post-prandial glucose obtained from about 100 mg of sitagliptin, from about 850 mg of metformin, or from about 1700 mg of metformin.

### BRIEF DESCRIPTION OF THE DRAWINGS

FIG. 1 shows the study design of the Composition A Phase I clinical trial.

FIG. 2 exemplifies a time course of plasma concentration of glucose (mean and standard error) before and after the administration of a single dose of placebo and Composition A in patients with type 2 diabetes (n=30) in a crossover study.

FIG. 3 exemplifies a time course of serum concentration of insulin (mean and standard error) before and after the administration of a single dose of placebo and Composition A in patients with type 2 diabetes (n=30) in a crossover study.

FIG. 4 exemplifies a graph of effect of single dose of sitagliptin 100 mg on plasma glucose (PG).

### DETAILED DESCRIPTION OF THE DISCLOSURE

Disclosed herein, in certain embodiments, are pharmaceutical compositions for the treatment of diabetes, comprising a low dose, therapeutically-effective amount of a DPP IV inhibitor (e.g., sitagliptin), a low dose, therapeutically-effective amount of a biguanide (e.g., metformin), and a low dose, therapeutically-effective amount of a sulfonylurea (e.g. glimepiride). In some embodiments, the dose of each component is below the lowest dose approved for the treatment of diabetes. In some embodiments, the low dose produces no or essentially no therapeutic effect as a monotherapy.

The present disclosure recognizes the technical effects of low-dose combination therapy set forth herein. Surprisingly, the combination of a low dose amount of a DPP IV inhibitor (e.g., sitagliptin), a low dose amount of a biguanide (e.g., metformin), and a low dose amount of a sulfonylurea (e.g., glimepiride) produces a therapeutic effect that is greater than the effect of any of the individual components administered as a monotherapy at the equivalent dosages. In some instances, the individual components produce no or essentially no therapeutic effect at equivalent dosages when administered as monotherapies.

The use of low dose amounts of each of the components results in beneficial effects, including but not limited to, avoiding or ameliorating negative side effects while retaining or improving benefits. Known side-effects of DPP IV inhibitors (e.g., sitagliptin), biguanides (e.g., metformin), and sulfonylureas (e.g. glimepiride) include upset stomach, nausea, and low blood sugar. Long-term side effects also include decreased absorption of vitamin B12 and lactic acidosis. Reducing these side-effects allows increased patient compliance and for the early introduction of combination therapy to improve therapeutic effects. Described herein in one aspect are low-dose combination compositions for the treatment of diabetes, including the initial or first-line treatment of diabetes.

### Certain Terminology

As used in the specification and appended claims, unless specified to the contrary, the following terms have the meaning indicated below.

As used herein and in the appended claims, the singular forms “a,” “and,” and “the” include plural referents unless the context clearly dictates otherwise. Thus, for example, reference to “an agent” includes a plurality of such agents, and reference to “the composition” includes reference to one or more compositions (or to a plurality of compositions) and equivalents thereof known to those skilled in the art, and so forth. When ranges are used herein for physical properties, such as molecular weight, or chemical properties, such as chemical formulae, all combinations and sub-combinations of ranges and specific embodiments therein are intended to be included. The term “about” when referring to a number or a numerical range means that the number or numerical range referred to is an approximation within experimental variability (or within statistical experimental error), and thus in some embodiments, the number or numerical range varies between 1% and 10% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

“Pharmaceutically acceptable salt” as used herein includes both acid and base addition salts. In some embodiments, the pharmaceutically acceptable salt of any one of the compounds described herein is the form approved for use by the US Food and Drug Administration. Preferred pharmaceutically acceptable salts of the compounds described herein are pharmaceutically acceptable acid addition salts and pharmaceutically acceptable base addition salts. “Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S.M. et al., “Pharmaceutical Salts,” Journal of Pharmaceutical Science, 66:1-19 (1997), which is hereby incorporated by reference in its entirety). Acid addition salts of basic compounds, in some embodiments, are prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts, in some embodiments, are formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

As used herein, “hydrates” are compounds that contain either stoichiometric or non-stoichiometric amounts of water, and, in some embodiments, are formed during the process of crystallization with water. Hydrates are meant to include the hydrates of any one of the compounds described herein that is approved for use by the US Food and Drug Administration.

The term “acceptable” with respect to a formulation, composition or ingredient, as used herein, means having no persistent detrimental effect on the general health of the subject being treated.

The terms “administer,” “administering,” “administration,” and the like, as used herein, refer to the methods that, in some embodiments, are used to enable delivery of compounds or compositions to the desired site of biological action. These methods include, but are not limited to, oral routes, intraduodenal routes, parenteral injection (including intravenous, subcutaneous, intraperitoneal, intramuscular, intravascular or infusion), topical, and rectal administration. Those of skill in the art are familiar with administration techniques that can be employed with the compounds and methods described herein. In some embodiments, the compounds and compositions described herein are administered orally.

The term “subject,” “patient” or “individual” encompasses mammals. Examples of mammals include, but are not limited to, any member of the Mammalian class: humans, non-human primates such as chimpanzees, and other apes and monkey species. In one aspect, the mammal is a human. None of “subject,” “patient,” or “individual” should be construed as requiring or not requiring the intervention of a medical professional.

As used herein, “treatment” or “treating” or “palliating” or “ameliorating” are used interchangeably herein. These terms refer to an approach for obtaining beneficial or desired results including but not limited to anti-diabetic effect, therapeutic benefit and/or a prophylactic benefit. By “therapeutic benefit” or “anti-diabetic effect” is meant eradication or amelioration of the underlying disorder being treated. A therapeutic benefit is achieved with the eradication or amelioration of one or more of the physiological symptoms associated with the underlying disorder (e.g., an improvement in: hyperglycemia, polyuria, polydipsia, polyphagia, diabetic dermadromes, etc.) such that an improvement is observed in the patient, notwithstanding that the patient, in some embodiments, is afflicted with the underlying disorder. Also, a therapeutic benefit is achieved with the eradication or amelioration of one or more of the complications associated with the underlying disorder (e.g., cardiovascular disease). For prophylactic benefit, the compositions, in some embodiments, are administered to a patient at risk of developing a particular disease, or to a patient reporting one or more of the physiological symptoms of a disease, even though a diagnosis of this disease, in some embodiments, has not been made.

The terms “diabetes” and “diabetes mellitus” are used interchangeably herein. These terms refers to type 1 diabetes mellitus, type 2 diabetes mellitus, complications of diabetes mellitus, and of neighboring disease states. As used herein, diabetes or diabetes mellitus (DM) refers to a group of metabolic disorders in which there are high blood sugar levels over a prolonged period.

### Triple Compositions

Described herein are pharmaceutical compositions comprising: (a) a dipeptidyl peptidase IV (DPP IV) inhibitor; (b) a biguanide; and (c) a sulfonylurea;
wherein (a), (b), and (c) are each at about 20% to about 75% of the lowest diabetes therapeutic dose (LDTD).

Described herein are pharmaceutical compositions consisting essentially of: (a) a dipeptidyl peptidase IV (DPP IV) inhibitor; (b) a biguanide; and (c) a sulfonylurea;
wherein (a), (b), and (c) are each at about 20% to about 75% of the lowest diabetes therapeutic dose (LDTD).

Described herein are pharmaceutical compositions comprising: a) a dipeptidyl peptidase IV (DPP IV) inhibitor; b) a biguanide; and c) a sulfonylurea;
wherein (a) and (b) are each at about 65%-75% of the lowest diabetes therapeutic dose (LDTD) for each of the (a) and (b), and (c) is at about 45%-55% of the lowest diabetes therapeutic dose (LDTD) for (c).

Described herein are pharmaceutical compositions consisting essentially of: a) a DPP IV inhibitor; b) a biguanide; and c) a sulfonylurea;
wherein (a) and (b) are each at about 65%-75% of the lowest diabetes therapeutic dose (LDTD) for each of the (a) and (b), and (c) is at about 45%-55% of the lowest diabetes therapeutic dose (LDTD) for (c).

Described herein are pharmaceutical compositions comprising (a) a DPP IV inhibitor; (b) a biguanide; and (c) a sulfonylurea; wherein (a), (b), and (c) are each at about 30% to about 70% of the lowest diabetes therapeutic dose (LDTD).

Described herein are pharmaceutical compositions consisting essentially of: (a) a DPP IV inhibitor; (b) a biguanide; and (c) a sulfonylurea; wherein (a), (b), and (c) are each at about 30% to about 70% of the lowest diabetes therapeutic dose (LDTD).

In some embodiments, the DPP IV inhibitor is a gliptin. In some embodiments, the DPP IV inhibitor is selected from sitagliptin, vildagliptin, saxagliptin, linagliptin, gemigliptin, anagliptin, teneligliptin, alogliptin, trelagliptin, omarigliptin, evogliptin, gosogliptin, dutogliptin, or the pharmaceutically acceptable salt or hydrate thereof.

In some embodiments, the DPP IV inhibitor is sitagliptin or the pharmaceutically acceptable salt thereof. In some embodiments, the DPP IV inhibitor is sitagliptin phosphate.

In some embodiments, the biguanide is metformin or the pharmaceutically acceptable salt or hydrate thereof. In some embodiments, the biguanide is metformin hydrochloride.

In some embodiments, the metformin is formulated for immediate release. In some embodiments, the metformin is formulated for slow release.

In some embodiments, the sulfonylurea is selected from acetohexamide, carbutamide, chlorpropamide, glycyclamide (tolhexamide), metahexamide, tolazamide, tolbutamide, glibenclamide (glyburide), glibornuride, gliclazide, glipizide, gliquidone, glisoxepide, glyclopyramide, glimepiride, or the pharmaceutically acceptable salt or hydrate thereof. In some embodiments, the sulfonylurea is glimepiride.

In some embodiments, the dose of each (a), (b), and (c) is from about 40% to about 75% of the lowest diabetes therapeutic dose (LDTD).

In some embodiments, the dose of each (a), (b), and (c) is from about 60% to about 75% of the lowest diabetes therapeutic dose (LDTD). In some embodiments, the dose of each (a), (b), and (c) is from about 65% to about 75% of the lowest diabetes therapeutic dose (LDTD). In some embodiments, the dose of each (a), (b), and (c) is about 70% of the lowest diabetes therapeutic dose (LDTD).

In some embodiments, the dose of each (a), (b), and (c) is from about 40% to about 70% of the lowest diabetes therapeutic dose (LDTD).

In some embodiments, the dose of each (a), (b), and (c) is from about 40% to about 60% of the lowest diabetes therapeutic dose (LDTD). In some embodiments, the dose of each (a), (b), and (c) is from about 45% to about 55% of the lowest diabetes therapeutic dose (LDTD). In some embodiments, the dose of each (a), (b), and (c) is about 50% of the lowest diabetes therapeutic dose (LDTD).

In some embodiments, the dose of each (a), (b), and (c) is from about 40% to about 70% of the lowest diabetes therapeutic dose (LDTD). In some embodiments, the dose of each (a), (b), and (c) is from about 35% to about 65% of the lowest diabetes therapeutic dose (LDTD). In some embodiments, the dose of each (a), (b), and (c) is from about 40% to about 60% of the lowest diabetes therapeutic dose (LDTD). In some embodiments, the dose of each (a), (b), and (c) is from about 45% to about 55% of lowest diabetes therapeutic dose (LDTD).

In some embodiments, the DPP IV inhibitor is about 50% of the lowest diabetes therapeutic dose (LDTD) for the DPP IV inhibitor. In some embodiments, the DPP IV inhibitor is sitagliptin or a pharmaceutically acceptable salt or hydrate thereof, and the dose of sitagliptin or the pharmaceutically acceptable salt or hydrate thereof is about 12.5 mg. In some embodiments, the DPP IV inhibitor is about 70% of the lowest diabetes therapeutic dose (LDTD) for the DPP IV inhibitor. In some embodiments, the DPP IV inhibitor is sitagliptin or a pharmaceutically acceptable salt or hydrate thereof, and the dose of sitagliptin or the pharmaceutically acceptable salt or hydrate thereof is about 17.5 mg.

In some embodiments, the biguanide is about 50% of the lowest diabetes therapeutic dose (LDTD) for the biguanide. In some embodiments, the biguanide is metformin hydrochloride, and the dose of metformin hydrochloride is about 250 mg. In some embodiments, the biguanide is about 70% of the lowest diabetes therapeutic dose (LDTD) for the biguanide. In some embodiments, the biguanide is metformin hydrochloride, and the dose of metformin hydrochloride is about 350 mg.

In some embodiments, the sulfonylurea is about 50% of the lowest diabetes therapeutic dose (LDTD) for the sulfonylurea. In some embodiments, the sulfonylurea is glimepiride or a pharmaceutically acceptable salt or hydrate thereof, and the dose of sulfonylurea is about 0.5 mg. In some embodiments, the sulfonylurea is about 70% of the lowest diabetes therapeutic dose (LDTD) for the sulfonylurea. In some embodiments, the sulfonylurea is glimepiride or a pharmaceutically acceptable salt or hydrate thereof, and the dose of sulfonylurea is about 0.7 mg.

In some embodiments, the DPP IV inhibitor is sitagliptin or a pharmaceutically acceptable salt or hydrate thereof, the biguanide is metformin or a pharmaceutically acceptable salt or hydrate thereof, and the sulfonylurea is glimepiride or a pharmaceutically acceptable salt or hydrate thereof. In some embodiments, the DPP IV inhibitor is sitagliptin, the biguanide is metformin, and the sulfonylurea is glimepiride.

In some embodiments, the dose of sitagliptin or a pharmaceutically acceptable salt or hydrate thereof is from about 5.0 mg to about 18.75 mg, the dose of metformin or a pharmaceutically acceptable salt or hydrate thereof is from about 100 mg to about 375 mg, and the dose of glimepiride or a pharmaceutically acceptable salt or hydrate thereof is from about 0.2 mg to about 0.75 mg. In some embodiments, the dose of sitagliptin or a pharmaceutically acceptable salt or hydrate thereof is from about 10 mg to about 16.25 mg, the dose of metformin or a pharmaceutically acceptable salt or hydrate thereof is from about 200 mg to about 325 mg, and the dose of glimepiride or a pharmaceutically acceptable salt or hydrate thereof is from about 0.4 mg to about 0.65 mg. In some embodiments, the dose of sitagliptin or a pharmaceutically acceptable salt or hydrate thereof is from about 10 mg to about 15 mg, the dose of metformin or a pharmaceutically acceptable salt or hydrate thereof is from about 200 mg to about 300 mg, and the dose of glimepiride is from about 0.4 mg to about 0.6 mg. In some embodiments, the dose of sitagliptin or a pharmaceutically acceptable salt or hydrate thereof is from about 11.25 mg to about 13.75 mg, the dose of metformin or a pharmaceutically acceptable salt or hydrate thereof is from about 225 mg to about 275 mg, and the dose of glimepiride or a pharmaceutically acceptable salt or hydrate thereof is from about 0.45 mg to about 0.55 mg. In some embodiments, the dose of sitagliptin or a pharmaceutically acceptable salt or hydrate thereof is about 12.5 mg, the dose of metformin or a pharmaceutically acceptable salt or hydrate thereof is about 250 mg, and the dose of glimepiride or a pharmaceutically acceptable salt or hydrate thereof is about 0.5 mg. In some embodiments, the dose of sitagliptin or a pharmaceutically acceptable salt or hydrate thereof is about 17.5 mg, the dose of metformin or a pharmaceutically acceptable salt or hydrate thereof is about 350 mg, and the dose of glimepiride or a pharmaceutically acceptable salt or hydrate thereof is about 0.5 mg.

In some embodiments, the dose of sitagliptin is from about 5.0 mg to about 18.75 mg, the dose of metformin is from about 100 mg to about 375 mg, and the dose of glimepiride is from about 0.2 mg to about 0.75 mg. In some embodiments, the dose of sitagliptin is from about 10 mg to about 16.25 mg, the dose of metformin is from about 200 mg to about 325 mg, and the dose of glimepiride is from about 0.4 mg to about 0.65 mg. In some embodiments, the dose of sitagliptin is from about 10 mg to about 15 mg, the dose of metformin is from about 200 mg to about 300 mg, and the dose of glimepiride is from about 0.4 mg to about 0.6 mg. In some embodiments, the dose of sitagliptin is from about 11.25 mg to about 13.75 mg, the dose of metformin is from about 225 mg to about 275 mg, and the dose of glimepiride is from about 0.45 mg to about 0.55 mg. In some embodiments, the dose of sitagliptin is about 12.5 mg, the dose of metformin is about 250 mg, and the dose of glimepiride is about 0.5 mg. In some embodiments, the dose of sitagliptin is about 17.5 mg, the dose of metformin is about 350 mg, and the dose of glimepiride is about 0.5 mg.

In some embodiments, the dose of each (a), (b), and (c) is from about 30% to about 40% of the lowest diabetes therapeutic dose (LDTD). In some embodiments, the dose of each (a), (b), and (c) is from about 30% to about 35% of the lowest diabetes therapeutic dose (LDTD).

In some embodiments, the sulfonylurea is about 33% of the lowest diabetes therapeutic dose (LDTD) for the sulfonylurea. In some embodiments, the sulfonylurea is glimepiride or a pharmaceutically acceptable salt or hydrate thereof, and the dose of sulfonylurea is about 0.33 mg. In some embodiments, the dose of sitagliptin or a pharmaceutically acceptable salt or hydrate thereof is about 8.25 mg, the dose of metformin or a pharmaceutically acceptable salt or hydrate thereof is about 165 mg, and the dose of glimepiride or a pharmaceutically acceptable salt or hydrate thereof is about 0.33 mg. In some embodiments, the dose of sitagliptin or a pharmaceutically acceptable salt or hydrate thereof is from about 7.5 mg to about 10 mg, the dose of metformin or a pharmaceutically acceptable salt or hydrate thereof is from about 150 mg to about 200 mg, and the dose of glimepiride or a pharmaceutically acceptable salt or hydrate thereof is from about 0.3 mg to about 0.4 mg.

In some embodiments, the dose of each (a), (b), and (c) is from about 20% to about 30% of the lowest diabetes therapeutic dose (LDTD). In some embodiments, the dose of each (a), (b), and (c) is from about 22% to about 28% of the lowest diabetes therapeutic dose (LDTD). In some embodiments, the sulfonylurea is about 25% of the lowest diabetes therapeutic dose (LDTD) for the sulfonylurea. In some embodiments, the sulfonylurea is glimepiride or a pharmaceutically acceptable salt or hydrate thereof, and the dose of sulfonylurea is about 0.25 mg. In some embodiments, the DPP IV inhibitor is about 25% of the lowest diabetes therapeutic dose (LDTD) for the DPP IV inhibitor. In some embodiments, the DPP IV inhibitor is sitagliptin or a pharmaceutically acceptable salt or hydrate thereof, and the dose of sitagliptin or the pharmaceutically acceptable salt or hydrate thereof is about 6.25 mg. In some embodiments, the biguanide is about 25% of the lowest diabetes therapeutic dose (LDTD) for the biguanide. In some embodiments, the biguanide is metformin hydrochloride, and the dose of metformin hydrochloride is about 125 mg.

In some embodiments, the pharmaceutical composition comprises: (a) sitagliptin as a DPP W; (b) metformin as a biguanide; and (c) glimepiride as a sulfonylurea. In some embodiments, the pharmaceutical composition comprises: (a) sitagliptin or a pharmaceutically acceptable salt or hydrate thereof as a DPP IV; (b) metformin or a pharmaceutically acceptable salt or hydrate thereof as a biguanide; and (c) glimepiride or a pharmaceutically acceptable salt or hydrate thereof as a sulfonylurea. In some embodiments, the dose of sitagliptin or a pharmaceutically acceptable salt or hydrate thereof is from about 5 mg to about 7.5 mg, the dose of metformin or a pharmaceutically acceptable salt or hydrate thereof is from about 100 mg to about 150 mg, and the dose of glimepiride or a pharmaceutically acceptable salt or hydrate thereof is from about 0.2 mg to about 0.3 mg. In some embodiments, the dose of sitagliptin or a pharmaceutically acceptable salt or hydrate thereof is about 6.25 mg, the dose of metformin or a pharmaceutically acceptable salt or hydrate thereof is about 150 mg, and the dose of glimepiride or a pharmaceutically acceptable salt or hydrate thereof is about 0.25 mg

In some embodiments, the biguanide is metformin or a pharmaceutically acceptable salt or hydrate thereof and the dose of metformin or the pharmaceutically acceptable salt or hydrate thereof is from about 325 mg to about 375 mg. In some embodiments, the biguanide is metformin and the dose of metformin is from about 325 mg to about 375 mg.

In some embodiments, the sulfonylurea is glimepiride or a pharmaceutically acceptable salt or hydrate thereof, and the dose of glimepiride or the pharmaceutically acceptable salt or hydrate thereof from about 0.45 mg to about 0.55 mg. In some embodiments, the sulfonylurea is glimepiride, and the dose of glimepiride from about 0.45 mg to about 0.55 mg.

In some embodiments, the DPP IV inhibitor is at about 70% of the lowest diabetes therapeutic dose (LDTD) for the DPP IV inhibitor.

In some embodiments, the biguanide is at about 70% of the lowest diabetes therapeutic dose (LDTD) for the biguanide.

In some embodiments, the sulfonylurea is at about 50% of the lowest diabetes therapeutic dose (LDTD) for the sulfonylurea.

In some embodiments, the DPP IV inhibitor is sitagliptin or a pharmaceutically acceptable salt or hydrate thereof, the biguanide is metformin or a pharmaceutically acceptable salt or hydrate thereof, and the sulfonylurea is glimepiride or a pharmaceutically acceptable salt or hydrate thereof. In some embodiments, the DPP IV inhibitor is sitagliptin, the biguanide is metformin, and the sulfonylurea is glimepiride.

In some embodiments, the dose of sitagliptin or a pharmaceutically acceptable salt or hydrate thereof is about 17.5 mg, the dose of metformin or a pharmaceutically acceptable salt or hydrate thereof is about 350 mg, and the dose of glimepiride or a pharmaceutically acceptable salt or hydrate thereof is about 0.5 mg. In some embodiments, the dose of sitagliptin is about 17.5 mg, the dose of metformin is about 350 mg, and the dose of glimepiride is about 0.5 mg.

In some embodiments, the DPP IV inhibitor is sitagliptin or a pharmaceutically acceptable salt or hydrate thereof and the dose of the sitagliptin or the pharmaceutically acceptable salt or hydrate thereof is about 17.5 mg. In some embodiments, the DPP IV inhibitor is sitagliptin and the dose of the sitagliptin is about 17.5 mg.

In some embodiments, the biguanide is metformin or a pharmaceutically acceptable salt or hydrate thereof and the dose of the metformin or the pharmaceutically acceptable salt or hydrate thereof is about 350 mg. In some embodiments, the biguanide is metformin and the dose of the metformin is about 350 mg.

In some embodiments, (a), (b), and (c) are provided in one formulation. In some embodiments, (a), (b), and (c) are each provided in a separate formulation. In some embodiments, two of the (a), (b), and (c) are provided in one formulation. In some embodiments, the pharmaceutical composition is in the form of pill, tablet or capsule. In some embodiments, the pharmaceutical composition is suitable for oral administration.

In some embodiments, the pharmaceutical compositions described herein comprise at least one pharmaceutically acceptable excipient. In some embodiments, the pharmaceutical compositions comprising (a) a DPP IV inhibitor; (b) a biguanide; and (c) a sulfonylurea described herein are essentially free of additional anti-hyperglycemic or anti-diabetic agents.

In some embodiments, the pharmaceutical composition comprises an anti-diabetic or anti-hyperglycemic combination of anti-diabetic active or anti-hyperglycemic agents, wherein the anti-diabetic or anti-hyperglycemic active agents consist of a DPP IV inhibitor; a biguanide; and a sulfonylurea.

Described herein are pharmaceutical compositions consisting essentially (a) a DPP IV inhibitor; (b) a biguanide; and (c) a sulfonylurea; wherein (a), (b), and (c) are each at about 20% to about 75% of the lowest diabetes therapeutic dose (LDTD).

In some embodiments, the pharmaceutical compositions disclosed herein achieve a significant anti-diabetic effect or therapeutic benefit in a subject with diabetes. In some embodiments, the pharmaceutical compositions disclosed herein achieve a significant anti-diabetic effect or therapeutic benefit in a subject with diabetes with minimum, insignificant, or no side effects. In some embodiments, the combination of the (a) a DPP IV inhibitor; (b) a biguanide; and (c) a sulfonylurea in the composition achieves a synergistic effect.

### DPP IV Inhibitors

As used herein, DPP IV inhibitors are compounds that block the enzyme dipeptidyl peptidase-4 (DPP IV) and reduce glucagon and blood glucose levels.

In some embodiments, the DPP IV inhibitor is a gliptin. In some embodiments, the DPP IV inhibitor is sitagliptin, vildagliptin, saxagliptin, linagliptin, gemigliptin, anagliptin, teneligliptin, alogliptin, trelagliptin, omarigliptin, evogliptin, gosogliptin, dutogliptin, or the pharmaceutically acceptable salt or hydrate thereof.

In some embodiments, the DPP IV inhibitor is sitagliptin, or the pharmaceutically acceptable salt or hydrate thereof. In some embodiments, the DPP IV inhibitor is sitagliptin phosphate, or a hydrate thereof. In some embodiments, the DPP IV inhibitor is vildagliptin, or the pharmaceutically acceptable salt or hydrate thereof. In some embodiments, the DPP IV inhibitor is saxagliptin, or the pharmaceutically acceptable salt or hydrate thereof. In some embodiments, the DPP IV inhibitor is linagliptin, or the pharmaceutically acceptable salt or hydrate thereof. In some embodiments, the DPP IV inhibitor is gemigliptin, or the pharmaceutically acceptable salt or hydrate thereof. In some embodiments, the DPP IV inhibitor is anagliptin, or the pharmaceutically acceptable salt or hydrate thereof. In some embodiments, the DPP IV inhibitor is teneligliptin, or the pharmaceutically acceptable salt or hydrate thereof. In some embodiments, the DPP IV inhibitor is alogliptin. In some embodiments, the DPP IV inhibitor is trelagliptin, or the pharmaceutically acceptable salt or hydrate thereof. In some embodiments, the DPP IV inhibitor is omarigliptin, or the pharmaceutically acceptable salt or hydrate thereof. In some embodiments, the DPP IV inhibitor is evogliptin, or the pharmaceutically acceptable salt or hydrate thereof. In some embodiments, the DPP IV inhibitor is gosogliptin, or the pharmaceutically acceptable salt or hydrate thereof. In some embodiments, the DPP IV inhibitor is dutogliptin, or the pharmaceutically acceptable salt or hydrate thereof.

### Biguanides

As used herein, biguanides are compounds that refer to a class of drugs that function as oral antihyperglycemic drugs used for diabetes mellitus or prediabetes treatment.

In some embodiments, the biguanide is metformin. In some embodiments, the biguanide is metformin hydrochloride, or a hydrate thereof.

### Sulfonylureas

As used herein, sulfonylureas are compounds that increase insulin release from the beta cells in the pancreas.

In some embodiments, the sulfonylurea is acetohexamide, carbutamide, chlorpropamide, glycyclamide (tolhexamide), metahexamide, tolazamide, tolbutamide, glibenclamide (glyburide), glibornuride, gliclazide, glipizide, gliquidone, glisoxepide, glyclopyramide, glimepiride, or the pharmaceutically acceptable salt or hydrate thereof. In some embodiments, the sulfonylurea is acetohexamide, or the pharmaceutically acceptable salt or hydrate thereof. In some embodiments, the sulfonylurea is carbutamide, or the pharmaceutically acceptable salt or hydrate thereof. In some embodiments, the sulfonylurea is chlorpropamide, or the pharmaceutically acceptable salt or hydrate thereof. In some embodiments, the sulfonylurea is glycyclamide (tolhexamide), or the pharmaceutically acceptable salt or hydrate thereof. In some embodiments, the sulfonylurea is metahexamide, or the pharmaceutically acceptable salt or hydrate thereof. In some embodiments, the sulfonylurea is tolazamide, or the pharmaceutically acceptable salt or hydrate thereof. In some embodiments, the sulfonylurea is tolbutamide, or the pharmaceutically acceptable salt or hydrate thereof. In some embodiments, the sulfonylurea is glibenclamide (glyburide), or the pharmaceutically acceptable salt or hydrate thereof. In some embodiments, the sulfonylurea is glibornuride, or the pharmaceutically acceptable salt or hydrate thereof. In some embodiments, the sulfonylurea is gliclazide, or the pharmaceutically acceptable salt or hydrate thereof. In some embodiments, the sulfonylurea is glipizide, or the pharmaceutically acceptable salt or hydrate thereof. In some embodiments, the sulfonylurea is glipizide, or the pharmaceutically acceptable salt or hydrate thereof. In some embodiments, the sulfonylurea is gliquidone, or the pharmaceutically acceptable salt or hydrate thereof. In some embodiments, the sulfonylurea is glisoxepide, or the pharmaceutically acceptable salt or hydrate thereof In some embodiments, the sulfonylurea is glyclopyramide, or the pharmaceutically acceptable salt or hydrate thereof. In some embodiments, the sulfonylurea is glyclopyramide, or the pharmaceutically acceptable salt or hydrate thereof. In some embodiments, the sulfonylurea is glimepiride, or the pharmaceutically acceptable salt or hydrate thereof.

### Lowest Diabetes Therapeutic Dose

As used herein, the lowest diabetes therapeutic dose (LDTD) refers to the lowest strength dose for the single agent for diabetes approved by the US Food and Drug Administration and is not marked as “discontinued” by the Orange Book database (world-wide web at address accessdata.fda.gov/scripts/cder/ob/) as of the filing date of this application. The lowest diabetes therapeutic dose does not include the lowest manufactured dose for cases wherein the lowest diabetes therapeutic dose is not the same as the lowest manufactured dose. Furthermore, the lowest diabetes therapeutic dose does not include the dose as recommended by a physician for cases wherein the lowest diabetes therapeutic dose is not the same dose as recommended by a physician. Further, the lowest diabetes dose of the DPP IV inhibitor, biguanide, and sulfonylurea, described herein refers to the dose of the form of DPP IV inhibitor, biguanide, and sulfonylurea approved for use by the US Food and Drug Administration, which includes the free base, pharmaceutically acceptable salt, or hydrate thereof.

In some embodiments, the dose of the DPP IV inhibitor is from about 20% to about 75% of the lowest diabetes therapeutic dose. In some embodiments, the dose of the DPP IV inhibitor is from about 20% to about 70% of the lowest diabetes therapeutic dose. In some embodiments, the dose of the DPP IV inhibitor is from about 20% to about 65% of the lowest diabetes therapeutic dose. In some embodiments, the dose of the DPP IV inhibitor is from about 20% to about 60% of the lowest diabetes therapeutic dose. In some embodiments, the dose of the DPP IV inhibitor is from about 20% to about 55% of the lowest diabetes therapeutic dose. In some embodiments, the dose of the DPP IV inhibitor is from about 20% to about 50% of the lowest diabetes therapeutic dose. In some embodiments, the dose of the DPP IV inhibitor is from about 20% to about 45% of the lowest diabetes therapeutic dose. In some embodiments, the dose of the DPP IV inhibitor is from about 20% to about 40% of the lowest diabetes therapeutic dose. In some embodiments, the dose of the DPP IV inhibitor is from about 20% to about 35% of the lowest diabetes therapeutic dose. In some embodiments, the dose of the DPP IV inhibitor is from about 20% to about 30% of the lowest diabetes therapeutic dose. In some embodiments, the dose of the DPP IV inhibitor is from about 20% to about 25% of the lowest diabetes therapeutic dose.

In some embodiments, the dose of the DPP IV inhibitor is from about 25% to about 75% of the lowest diabetes therapeutic dose. In some embodiments, the dose of the DPP IV inhibitor is from about 25% to about 70% of the lowest diabetes therapeutic dose. In some embodiments, the dose of the DPP IV inhibitor is from about 25% to about 65% of the lowest diabetes therapeutic dose. In some embodiments, the dose of the DPP IV inhibitor is from about 25% to about 60% of the lowest diabetes therapeutic dose. In some embodiments, the dose of the DPP IV inhibitor is from about 25% to about 55% of the lowest diabetes therapeutic dose. In some embodiments, the dose of the DPP IV inhibitor is from about 25% to about 50% of the lowest diabetes therapeutic dose. In some embodiments, the dose of the DPP IV inhibitor is from about 25% to about 45% of the lowest diabetes therapeutic dose. In some embodiments, the dose of the DPP IV inhibitor is from about 25% to about 40% of the lowest diabetes therapeutic dose. In some embodiments, the dose of the DPP IV inhibitor is from about 25% to about 35% of the lowest diabetes therapeutic dose. In some embodiments, the dose of the DPP IV inhibitor is from about 25% to about 30% of the lowest diabetes therapeutic dose.

In some embodiments, the dose of the DPP IV inhibitor is from about 30% to about 75% of the lowest diabetes therapeutic dose. In some embodiments, the dose of the DPP W inhibitor is from about 30% to about 70% of the lowest diabetes therapeutic dose. In some embodiments, the dose of the DPP IV inhibitor is from about 30% to about 65% of the lowest diabetes therapeutic dose. In some embodiments, the dose of the DPP IV inhibitor is from about 30% to about 60% of the lowest diabetes therapeutic dose. In some embodiments, the dose of the DPP IV inhibitor is from about 30% to about 55% of the lowest diabetes therapeutic dose. In some embodiments, the dose of the DPP IV inhibitor is from about 30% to about 50% of the lowest diabetes therapeutic dose. In some embodiments, the dose of the DPP IV inhibitor is from about 30% to about 45% of the lowest diabetes therapeutic dose. In some embodiments, the dose of the DPP IV inhibitor is from about 30% to about 40% of the lowest diabetes therapeutic dose. In some embodiments, the dose of the DPP IV inhibitor is from about 30% to about 35% of the lowest diabetes therapeutic dose.

In some embodiments, the dose of the DPP IV inhibitor is from about 35% to about 75% of the lowest diabetes therapeutic dose. In some embodiments, the dose of the DPP W inhibitor is from about 35% to about 70% of the lowest diabetes therapeutic dose. In some embodiments, the dose of the DPP IV inhibitor is from about 35% to about 65% of the lowest diabetes therapeutic dose. In some embodiments, the dose of the DPP IV inhibitor is from about 35% to about 60% of the lowest diabetes therapeutic dose. In some embodiments, the dose of the DPP IV inhibitor is from about 35% to about 55% of the lowest diabetes therapeutic dose. In some embodiments, the dose of the DPP IV inhibitor is from about 35% to about 50% of the lowest diabetes therapeutic dose. In some embodiments, the dose of the DPP IV inhibitor is from about 35% to about 45% of the lowest diabetes therapeutic dose. In some embodiments, the dose of the DPP IV inhibitor is from about 35% to about 40% of the lowest diabetes therapeutic dose

In some embodiments, the dose of the DPP IV inhibitor is from about 40% to about 75% of the lowest diabetes therapeutic dose. In some embodiments, the dose of the DPP IV inhibitor is from about 40% to about 70% of the lowest diabetes therapeutic dose. In some embodiments, the dose of the DPP IV inhibitor is from about 40% to about 65% of the lowest diabetes therapeutic dose. In some embodiments, the dose of the DPP IV inhibitor is from about 40% to about 60% of the lowest diabetes therapeutic dose. In some embodiments, the dose of the DPP IV inhibitor is from about 40% to about 55% of the lowest diabetes therapeutic dose. In some embodiments, the dose of the DPP IV inhibitor is from about 40% to about 50% of the lowest diabetes therapeutic dose. In some embodiments, the dose of the DPP IV inhibitor is from about 40% to about 45% of the lowest diabetes therapeutic dose.

In some embodiments, the dose of the DPP IV inhibitor is from about 45% to about 75% of the lowest diabetes therapeutic dose. In some embodiments, the dose of the DPP IV inhibitor is from about 45% to about 70% of the lowest diabetes therapeutic dose. In some embodiments, the dose of the DPP IV inhibitor is from about 45% to about 65% of the lowest diabetes therapeutic dose. In some embodiments, the dose of the DPP IV inhibitor is from about 45% to about 60% of the lowest diabetes therapeutic dose. In some embodiments, the dose of the DPP IV inhibitor is from about 45% to about 55% of the lowest diabetes therapeutic dose. In some embodiments, the dose of the DPP IV inhibitor is from about 45% to about 50% of the lowest diabetes therapeutic dose.

In some embodiments, the dose of the DPP IV inhibitor is from about 50% to about 75% of the lowest diabetes therapeutic dose. In some embodiments, the dose of the DPP IV inhibitor is from about 50% to about 70% of the lowest diabetes therapeutic dose. In some embodiments, the dose of the DPP IV inhibitor is from about 50% to about 65% of the lowest diabetes therapeutic dose. In some embodiments, the dose of the DPP IV inhibitor is from about 50% to about 60% of the lowest diabetes therapeutic dose. In some embodiments, the dose of the DPP IV inhibitor is from about 50% to about 55% of the lowest diabetes therapeutic dose.

In some embodiments, the dose of the DPP IV inhibitor is from about 55% to about 75% of the lowest diabetes therapeutic dose. In some embodiments, the dose of the DPP IV inhibitor is from about 55% to about 70% of the lowest diabetes therapeutic dose. In some embodiments, the dose of the DPP IV inhibitor is from about 55% to about 65% of the lowest diabetes therapeutic dose. In some embodiments, the dose of the DPP IV inhibitor is from about 55% to about 60% of the lowest diabetes therapeutic dose.

In some embodiments, the dose of the DPP IV inhibitor is from about 60% to about 75% of the lowest diabetes therapeutic dose. In some embodiments, the dose of the DPP IV inhibitor is from about 60% to about 70% of the lowest diabetes therapeutic dose. In some embodiments, the dose of the DPP IV inhibitor is from about 60% to about 65% of the lowest diabetes therapeutic dose.

In some embodiments, the dose of the DPP IV inhibitor is from about 65% to about 75% of the lowest diabetes therapeutic dose. In some embodiments, the dose of the DPP IV inhibitor is from about 65% to about 70% of the lowest diabetes therapeutic dose.

In some embodiments, the dose of the DPP IV inhibitor is from about 70% to about 75% of the lowest diabetes therapeutic dose.

In some embodiments, the dose of the biguanide is from about 20% to about 75% of the lowest diabetes therapeutic dose. In some embodiments, the dose of the biguanide is from about 20% to about 70% of the lowest diabetes therapeutic dose. In some embodiments, the dose of the biguanide is from about 20% to about 65% of the lowest diabetes therapeutic dose. In some embodiments, the dose of the biguanide is from about 20% to about 60% of the lowest diabetes therapeutic dose. In some embodiments, the dose of the biguanide is from about 20% to about 55% of the lowest diabetes therapeutic dose. In some embodiments, the dose of the biguanide is from about 20% to about 50% of the lowest diabetes therapeutic dose. In some embodiments, the dose of the biguanide is from about 20% to about 45% of the lowest diabetes therapeutic dose. In some embodiments, the dose of the biguanide is from about 20% to about 40% of the lowest diabetes therapeutic dose. In some embodiments, the dose of the biguanide is from about 20% to about 35% of the lowest diabetes therapeutic dose. In some embodiments, the dose of the biguanide is from about 20% to about 30% of the lowest diabetes therapeutic dose. In some embodiments, the dose of the biguanide is from about 20% to about 25% of the lowest diabetes therapeutic dose.

In some embodiments, the dose of the biguanide is from about 25% to about 75% of the lowest diabetes therapeutic dose. In some embodiments, the dose of the biguanide is from about 25% to about 70% of the lowest diabetes therapeutic dose. In some embodiments, the dose of the biguanide is from about 25% to about 65% of the lowest diabetes therapeutic dose. In some embodiments, the dose of the biguanide is from about 25% to about 60% of the lowest diabetes therapeutic dose. In some embodiments, the dose of the biguanide is from about 25% to about 55% of the lowest diabetes therapeutic dose. In some embodiments, the dose of the biguanide is from about 25% to about 50% of the lowest diabetes therapeutic dose. In some embodiments, the dose of the biguanide is from about 25% to about 45% of the lowest diabetes therapeutic dose. In some embodiments, the dose of the biguanide is from about 25% to about 40% of the lowest diabetes therapeutic dose. In some embodiments, the dose of the biguanide is from about 25% to about 35% of the lowest diabetes therapeutic dose. In some embodiments, the dose of the biguanide is from about 25% to about 30% of the lowest diabetes therapeutic dose.

In some embodiments, the dose of the biguanide is from about 30% to about 75% of the lowest diabetes therapeutic dose. In some embodiments, the dose of the biguanide is from about 30% to about 70% of the lowest diabetes therapeutic dose. In some embodiments, the dose of the biguanide is from about 30% to about 65% of the lowest diabetes therapeutic dose. In some embodiments, the dose of the biguanide is from about 30% to about 60% of the lowest diabetes therapeutic dose. In some embodiments, the dose of the biguanide is from about 30% to about 55% of the lowest diabetes therapeutic dose. In some embodiments, the dose of the biguanide is from about 30% to about 50% of the lowest diabetes therapeutic dose. In some embodiments, the dose of the biguanide is from about 30% to about 45% of the lowest diabetes therapeutic dose. In some embodiments, the dose of the biguanide is from about 30% to about 40% of the lowest diabetes therapeutic dose. In some embodiments, the dose of the biguanide is from about 30% to about 35% of the lowest diabetes therapeutic dose.

In some embodiments, the dose of the biguanide is from about 35% to about 75% of the lowest diabetes therapeutic dose. In some embodiments, the dose of the biguanide is from about 35% to about 70% of the lowest diabetes therapeutic dose. In some embodiments, the dose of the biguanide is from about 35% to about 65% of the lowest diabetes therapeutic dose. In some embodiments, the dose of the biguanide is from about 35% to about 60% of the lowest diabetes therapeutic dose. In some embodiments, the dose of the biguanide is from about 35% to about 55% of the lowest diabetes therapeutic dose. In some embodiments, the dose of the biguanide is from about 35% to about 50% of the lowest diabetes therapeutic dose. In some embodiments, the dose of the biguanide is from about 35% to about 45% of the lowest diabetes therapeutic dose. In some embodiments, the dose of the biguanide is from about 35% to about 40% of the lowest diabetes therapeutic dose.

In some embodiments, the dose of the biguanide is from about 40% to about 75% of the lowest diabetes therapeutic dose. In some embodiments, the dose of the biguanide is from about 40% to about 70% of the lowest diabetes therapeutic dose. In some embodiments, the dose of the biguanide is from about 40% to about 65% of the lowest diabetes therapeutic dose. In some embodiments, the dose of the biguanide is from about 40% to about 60% of the lowest diabetes therapeutic dose. In some embodiments, the dose of the biguanide is from about 40% to about 55% of the lowest diabetes therapeutic dose. In some embodiments, the dose of the biguanide is from about 40% to about 50% of the lowest diabetes therapeutic dose. In some embodiments, the dose of the biguanide is from about 40% to about 45% of the lowest diabetes therapeutic dose.

In some embodiments, the dose of the biguanide is from about 45% to about 75% of the lowest diabetes therapeutic dose. In some embodiments, the dose of the biguanide is from about 45% to about 70% of the lowest diabetes therapeutic dose. In some embodiments, the dose of the biguanide is from about 45% to about 65% of the lowest diabetes therapeutic dose. In some embodiments, the dose of the biguanide is from about 45% to about 60% of the lowest diabetes therapeutic dose. In some embodiments, the dose of the biguanide is from about 45% to about 55% of the lowest diabetes therapeutic dose. In some embodiments, the dose of the biguanide is from about 45% to about 50% of the lowest diabetes therapeutic dose.

In some embodiments, the dose of the biguanide is from about 50% to about 75% of the lowest diabetes therapeutic dose. In some embodiments, the dose of the biguanide is from about 50% to about 70% of the lowest diabetes therapeutic dose. In some embodiments, the dose of the biguanide is from about 50% to about 65% of the lowest diabetes therapeutic dose. In some embodiments, the dose of the biguanide is from about 50% to about 60% of the lowest diabetes therapeutic dose. In some embodiments, the dose of the biguanide is from about 50% to about 55% of the lowest diabetes therapeutic dose.

In some embodiments, the dose of the biguanide is from about 55% to about 75% of the lowest diabetes therapeutic dose. In some embodiments, the dose of the biguanide is from about 55% to about 70% of the lowest diabetes therapeutic dose. In some embodiments, the dose of the biguanide is from about 55% to about 65% of the lowest diabetes therapeutic dose. In some embodiments, the dose of the biguanide is from about 55% to about 60% of the lowest diabetes therapeutic dose.

In some embodiments, the dose of the biguanide is from about 60% to about 75% of the lowest diabetes therapeutic dose. In some embodiments, the dose of the biguanide is from about 60% to about 70% of the lowest diabetes therapeutic dose. In some embodiments, the dose of the biguanide is from about 60% to about 65% of the lowest diabetes therapeutic dose.

In some embodiments, the dose of the biguanide is from about 65% to about 75% of the lowest diabetes therapeutic dose. In some embodiments, the dose of the biguanide is from about 65% to about 70% of the lowest diabetes therapeutic dose.

In some embodiments, the dose of the biguanide is from about 70% to about 75% of the lowest diabetes therapeutic dose.

In some embodiments, the dose of the sulfonylurea is from about 20% to about 75% of the lowest diabetes therapeutic dose. In some embodiments, the dose of the sulfonylurea is from about 20% to about 70% of the lowest diabetes therapeutic dose. In some embodiments, the dose of the sulfonylurea is from about 20% to about 65% of the lowest diabetes therapeutic dose. In some embodiments, the dose of the sulfonylurea is from about 20% to about 60% of the lowest diabetes therapeutic dose. In some embodiments, the dose of the sulfonylurea is from about 20% to about 55% of the lowest diabetes therapeutic dose. In some embodiments, the dose of the sulfonylurea is from about 20% to about 50% of the lowest diabetes therapeutic dose. In some embodiments, the dose of the sulfonylurea is from about 20% to about 45% of the lowest diabetes therapeutic dose. In some embodiments, the dose of the sulfonylurea is from about 20% to about 40% of the lowest diabetes therapeutic dose. In some embodiments, the dose of the Sulfonylurea is from about 20% to about 35% of the lowest diabetes therapeutic dose. In some embodiments, the dose of the sulfonylurea is from about 20% to about 30% of the lowest diabetes therapeutic dose. In some embodiments, the dose of the sulfonylurea is from about 20% to about 25% of the lowest diabetes therapeutic dose.

In some embodiments, the dose of the sulfonylurea is from about 25% to about 75% of the lowest diabetes therapeutic dose. In some embodiments, the dose of the sulfonylurea is from about 25% to about 70% of the lowest diabetes therapeutic dose. In some embodiments, the dose of the sulfonylurea is from about 25% to about 65% of the lowest diabetes therapeutic dose. In some embodiments, the dose of the sulfonylurea is from about 25% to about 60% of the lowest diabetes therapeutic dose. In some embodiments, the dose of the sulfonylurea is from about 25% to about 55% of the lowest diabetes therapeutic dose. In some embodiments, the dose of the sulfonylurea is from about 25% to about 50% of the lowest diabetes therapeutic dose. In some embodiments, the dose of the sulfonylurea is from about 25% to about 45% of the lowest diabetes therapeutic dose. In some embodiments, the dose of the sulfonylurea is from about 25% to about 40% of the lowest diabetes therapeutic dose. In some embodiments, the dose of the sulfonylurea is from about 25% to about 35% of the lowest diabetes therapeutic dose. In some embodiments, the dose of the sulfonylurea is from about 25% to about 30% of the lowest diabetes therapeutic dose.

In some embodiments, the dose of the sulfonylurea is from about 30% to about 75% of the lowest diabetes therapeutic dose. In some embodiments, the dose of the sulfonylurea is from about 30% to about 70% of the lowest diabetes therapeutic dose. In some embodiments, the dose of the sulfonylurea is from about 30% to about 65% of the lowest diabetes therapeutic dose. In some embodiments, the dose of the sulfonylurea is from about 30% to about 60% of the lowest diabetes therapeutic dose. In some embodiments, the dose of the sulfonylurea is from about 30% to about 55% of the lowest diabetes therapeutic dose. In some embodiments, the dose of the sulfonylurea is from about 30% to about 50% of the lowest diabetes therapeutic dose. In some embodiments, the dose of the Sulfonylurea is from about 30% to about 45% of the lowest diabetes therapeutic dose. In some embodiments, the dose of the sulfonylurea is from about 30% to about 40% of the lowest diabetes therapeutic dose. In some embodiments, the dose of the sulfonylurea is from about 30% to about 35% of the lowest diabetes therapeutic dose.

In some embodiments, the dose of the sulfonylurea is from about 35% to about 75% of the lowest diabetes therapeutic dose. In some embodiments, the dose of the sulfonylurea is from about 35% to about 70% of the lowest diabetes therapeutic dose. In some embodiments, the dose of the sulfonylurea is from about 35% to about 65% of the lowest diabetes therapeutic dose. In some embodiments, the dose of the sulfonylurea is from about 35% to about 60% of the lowest diabetes therapeutic dose. In some embodiments, the dose of the sulfonylurea is from about 35% to about 55% of the lowest diabetes therapeutic dose. In some embodiments, the dose of the Sulfonylurea is from about 35% to about 50% of the lowest diabetes therapeutic dose. In some embodiments, the dose of the sulfonylurea is from about 35% to about 45% of the lowest diabetes therapeutic dose. In some embodiments, the dose of the sulfonylurea is from about 35% to about 40% of the lowest diabetes therapeutic dose

In some embodiments, the dose of the sulfonylurea is from about 40% to about 75% of the lowest diabetes therapeutic dose. In some embodiments, the dose of the sulfonylurea is from about 40% to about 70% of the lowest diabetes therapeutic dose. In some embodiments, the dose of the sulfonylurea is from about 40% to about 65% of the lowest diabetes therapeutic dose. In some embodiments, the dose of the sulfonylurea is from about 40% to about 60% of the lowest diabetes therapeutic dose. In some embodiments, the dose of the sulfonylurea is from about 40% to about 55% of the lowest diabetes therapeutic dose. In some embodiments, the dose of the sulfonylurea is from about 40% to about 50% of the lowest diabetes therapeutic dose. In some embodiments, the dose of the sulfonylurea is from about 40% to about 45% of the lowest diabetes therapeutic dose.

In some embodiments, the dose of the sulfonylurea is from about 45% to about 75% of the lowest diabetes therapeutic dose. In some embodiments, the dose of the sulfonylurea is from about 45% to about 70% of the lowest diabetes therapeutic dose. In some embodiments, the dose of the Sulfonylurea is from about 45% to about 65% of the lowest diabetes therapeutic dose. In some embodiments, the dose of the sulfonylurea is from about 45% to about 60% of the lowest diabetes therapeutic dose. In some embodiments, the dose of the sulfonylurea is from about 45% to about 55% of the lowest diabetes therapeutic dose. In some embodiments, the dose of the sulfonylurea is from about 45% to about 50% of the lowest diabetes therapeutic dose.

In some embodiments, the dose of the sulfonylurea is from about 50% to about 75% of the lowest diabetes therapeutic dose. In some embodiments, the dose of the sulfonylurea is from about 50% to about 70% of the lowest diabetes therapeutic dose. In some embodiments, the dose of the sulfonylurea is from about 50% to about 65% of the lowest diabetes therapeutic dose. In some embodiments, the dose of the sulfonylurea is from about 50% to about 60% of the lowest diabetes therapeutic dose. In some embodiments, the dose of the sulfonylurea is from about 50% to about 55% of the lowest diabetes therapeutic dose.

In some embodiments, the dose of the sulfonylurea is from about 55% to about 75% of the lowest diabetes therapeutic dose. In some embodiments, the dose of the sulfonylurea is from about 55% to about 70% of the lowest diabetes therapeutic dose. In some embodiments, the dose of the sulfonylurea is from about 55% to about 65% of the lowest diabetes therapeutic dose. In some embodiments, the dose of the sulfonylurea is from about 55% to about 60% of the lowest diabetes therapeutic dose.

In some embodiments, the dose of the sulfonylurea is from about 60% to about 75% of the lowest diabetes therapeutic dose. In some embodiments, the dose of the sulfonylurea is from about 60% to about 70% of the lowest diabetes therapeutic dose. In some embodiments, the dose of the Sulfonylurea is from about 60% to about 65% of the lowest diabetes therapeutic dose.

In some embodiments, the dose of the Sulfonylurea is from about 65% to about 75% of the lowest diabetes therapeutic dose. In some embodiments, the dose of the sulfonylurea is from about 65% to about 70% of the lowest diabetes therapeutic dose.

In some embodiments, the dose of the sulfonylurea is from about 70% to about 75% of the lowest diabetes therapeutic dose.

In some embodiments, the dose of the DPP IV inhibitor is from about 20% to about 50% of the lowest diabetes therapeutic dose. In some embodiments, the dose of the DPP IV inhibitor is from about 20% to about 45% of the lowest diabetes therapeutic dose. In some embodiments, the dose of the DPP IV inhibitor is from about 20% to about 40% of the lowest diabetes therapeutic dose. In some embodiments, the dose of the DPP IV inhibitor is from about 20% to about 35% of the lowest diabetes therapeutic dose. In some embodiments, the dose of the DPP IV inhibitor is from about 20% to about 30% of the lowest diabetes therapeutic dose. In some embodiments, the dose of the DPP IV inhibitor is from about 20% to about 25% of the lowest diabetes therapeutic dose.

In some embodiments, the dose of the DPP IV inhibitor is from about 25% to about 50% of the lowest diabetes therapeutic dose. In some embodiments, the dose of the DPP IV inhibitor is from about 25% to about 45% of the lowest diabetes therapeutic dose. In some embodiments, the dose of the DPP IV inhibitor is from about 25% to about 40% of the lowest diabetes therapeutic dose. In some embodiments, the dose of the DPP IV inhibitor is from about 25% to about 35% of the lowest diabetes therapeutic dose. In some embodiments, the dose of the DPP IV inhibitor is from about 25% to about 30% of the lowest diabetes therapeutic dose.

In some embodiments, the dose of the DPP IV inhibitor is from about 30% to about 50% of the lowest diabetes therapeutic dose. In some embodiments, the dose of the DPP IV inhibitor is from about 30% to about 45% of the lowest diabetes therapeutic dose. In some embodiments, the dose of the DPP IV inhibitor is from about 30% to about 40% of the lowest diabetes therapeutic dose. In some embodiments, the dose of the DPP IV inhibitor is from about 30% to about 35% of the lowest diabetes therapeutic dose.

In some embodiments, the dose of the DPP IV inhibitor is from about 35% to about 50% of the lowest diabetes therapeutic dose. In some embodiments, the dose of the DPP IV inhibitor is from about 35% to about 45% of the lowest diabetes therapeutic dose. In some embodiments, the dose of the DPP IV inhibitor is from about 35% to about 40% of the lowest diabetes therapeutic dose.

In some embodiments, the dose of the DPP IV inhibitor is from about 40% to about 50% of the lowest diabetes therapeutic dose. In some embodiments, the dose of the DPP W inhibitor is from about 45% to about 50% of the lowest diabetes therapeutic dose.

In some embodiments, the dose of the biguanide is from about 20% to about 50% of the lowest diabetes therapeutic dose. In some embodiments, the dose of the biguanide is from about 20% to about 45% of the lowest diabetes therapeutic dose. In some embodiments, the dose of the biguanide is from about 20% to about 40% of the lowest diabetes therapeutic dose. In some embodiments, the dose of the biguanide is from about 20% to about 35% of the lowest diabetes therapeutic dose. In some embodiments, the dose of the biguanide is from about 20% to about 30% of the lowest diabetes therapeutic dose. In some embodiments, the dose of the biguanide is from about 20% to about 25% of the lowest diabetes therapeutic dose.

In some embodiments, the dose of the biguanide is from about 25% to about 50% of the lowest diabetes therapeutic dose. In some embodiments, the dose of the biguanide is from about 25% to about 45% of the lowest diabetes therapeutic dose. In some embodiments, the dose of the biguanide is from about 25% to about 40% of the lowest diabetes therapeutic dose. In some embodiments, the dose of the biguanide is from about 25% to about 35% of the lowest diabetes therapeutic dose. In some embodiments, the dose of the biguanide is from about 25% to about 30% of the lowest diabetes therapeutic dose.

In some embodiments, the dose of the biguanide is from about 30% to about 50% of the lowest diabetes therapeutic dose. In some embodiments, the dose of the biguanide is from about 30% to about 45% of the lowest diabetes therapeutic dose. In some embodiments, the dose of the biguanide is from about 30% to about 40% of the lowest diabetes therapeutic dose. In some embodiments, the dose of the biguanide is from about 30% to about 35% of the lowest diabetes therapeutic dose.

In some embodiments, the dose of the biguanide is from about 35% to about 50% of the lowest diabetes therapeutic dose. In some embodiments, the dose of the biguanide is from about 35% to about 45% of the lowest diabetes therapeutic dose. In some embodiments, the dose of the biguanide is from about 35% to about 40% of the lowest diabetes therapeutic dose.

In some embodiments, the dose of the biguanide is from about 40% to about 50% of the lowest diabetes therapeutic dose. In some embodiments, the dose of the biguanide is from about 45% to about 50% of the lowest diabetes therapeutic dose.

In some embodiments, the dose of the sulfonylurea is from about 20% to about 50% of the lowest diabetes therapeutic dose. In some embodiments, the dose of the sulfonylurea is from about 20% to about 45% of the lowest diabetes therapeutic dose. In some embodiments, the dose of the is from about 20% to about 40% of the lowest diabetes therapeutic dose. In some embodiments, the dose of the sulfonylurea is from about 20% to about 35% of the lowest diabetes therapeutic dose. In some embodiments, the dose of the sulfonylurea is from about 20% to about 30% of the lowest diabetes therapeutic dose. In some embodiments, the dose of the Sulfonylurea is from about 20% to about 25% of the lowest diabetes therapeutic dose.

In some embodiments, the dose of the sulfonylurea is from about 25% to about 50% of the lowest diabetes therapeutic dose. In some embodiments, the dose of the sulfonylurea is from about 25% to about 45% of the lowest diabetes therapeutic dose. In some embodiments, the dose of the sulfonylurea is from about 25% to about 40% of the lowest diabetes therapeutic dose. In some embodiments, the dose of the Sulfonylurea is from about 25% to about 35% of the lowest diabetes therapeutic dose. In some embodiments, the dose of the sulfonylurea is from about 25% to about 30% of the lowest diabetes therapeutic dose.

In some embodiments, the dose of the sulfonylurea is from about 30% to about 50% of the lowest diabetes therapeutic dose. In some embodiments, the dose of the sulfonylurea is from about 30% to about 45% of the lowest diabetes therapeutic dose. In some embodiments, the dose of the sulfonylurea is from about 30% to about 40% of the lowest diabetes therapeutic dose. In some embodiments, the dose of the sulfonylurea is from about 30% to about 35% of the lowest diabetes therapeutic dose.

In some embodiments, the dose of the sulfonylurea is from about 35% to about 50% of the lowest diabetes therapeutic dose. In some embodiments, the dose of the sulfonylurea is from about 35% to about 45% of the lowest diabetes therapeutic dose. In some embodiments, the dose of the sulfonylurea is from about 35% to about 40% of the lowest diabetes therapeutic dose. In some embodiments, the dose of the sulfonylurea is from about 40% to about 50% of the lowest diabetes therapeutic dose. In some embodiments, the dose of the sulfonylurea is from about 45% to about 50% of the lowest diabetes therapeutic dose.

In some embodiments, the dose of each of the DPP IV inhibitor, the biguanide, and the sulfonylurea is from about 20% to about 40% of the lowest diabetes therapeutic dose.

In some embodiments, the dose of each of the DPP IV inhibitor, the biguanide, and the sulfonylurea is from about 20% to about 30% of the lowest diabetes therapeutic dose.

In some embodiments, the dose of the DPP IV inhibitor is about 20%, about 21%, about 22, about 23%, about 24%, about 25%, about 26%, about 27%, about 28%, about 29%, or about 30% of the lowest diabetes therapeutic dose. In some embodiments, the dose of the DPP IV inhibitor is about 25% of the lowest diabetes therapeutic dose. In some embodiments, the DPP IV inhibitor is sitagliptin. In some embodiments, the dose of sitagliptin is about 5.0, about 5.25, about 5.5, about 5.75, about 6.0, about 6.25, about 6.5, about 6.75, about 7.0, about 7.25, or about 7.5 mg.

In some embodiments, the dose of the biguanide inhibitor is about 20%, about 21%, about 22, about 23%, about 24%, about 25%, about 26%, about 27%, about 28%, about 29%, or about 30% of the lowest diabetes therapeutic dose. In some embodiments, the dose of the biguanide is about 25% of the lowest diabetes therapeutic dose. In some embodiments, the biguanide is metformin or metformin hydrochloride. In some embodiments, the dose of metformin or metformin hydrochloride is about 100, about 105, about, 110, about 115, about 120, about 125, about 130, about 135, about 140, about 145, or about 150 mg.

In some embodiments, the dose of the sulfonylurea is about 20%, about 21%, about 22, about 23%, about 24%, about 25%, about 26%, about 27%, about 28%, about 29%, or about 30% of the lowest diabetes therapeutic dose. In some embodiments, the dose of the sulfonylurea is about 25% of the lowest diabetes therapeutic dose. In some embodiments, the sulfonylurea is glimepiride. In some embodiments, the dose of glimepiride is about 0.20, about 0.21, about 0.22, about 0.23, about 0.24, about 0.25, about 0.26, about 0.27, about 0.28, about 0.29, or about 0.30 mg.

In some embodiments, the lowest diabetes therapeutic dose (LDTD) and the corresponding proposed dose and proposed dose range for the following compounds are as described in Table 1.

TABLE 1

| | | | Proposed |
|---|---|---|---|
| | Lowest Diabetes | Proposed | Dose Range |
| | Therapeutic Dose | Dose (mg) | 20%-30% |
| Agent | (LDTD) (mg) | (25% LDTD) | LDTD (mg) |
| sitagliptin | 25 | 6.25 | 5-7.5 |
| vildagliptin | 50 | 12.5 | 10-15 |
| saxagliptin | 2.5 | 0.625 | 0.5-0.75 |
| linagliptin | 5 | 1.25 | 1-1.5 |
| alogliptin | 6.25 | 1.5625 | 1.25-1.875 |
| metformin | 500 | 125 | 100-150 |
| acetohexamide | 250 | 62.5 | 50-75 |
| tolazamide | 100 | 25 | 20-30 |
| tolbutamide | 250 | 62.5 | 50-75 |
| glibenclamide | 1.25 | 0.3125 | 0.25-0.375 |
| (glyburide) | | | |
| gliclazide | 30 | 7.5 | 6-9 |
| glipizide | 2.5 | 0.625 | 0.5-0.75 |
| gliquidone | 30 | 7.5 | 6-9 |
| glimepiride | 1 | 0.25 | 0.2-0.3 |

In some embodiments, the pharmaceutical composition comprises: (a) sitagliptin as a DPP W inhibitor; (b) metformin as a biguanide; and (c) glimepiride as a sulfonylurea. In some embodiments, the dose of sitagliptin is from about 5 mg to about 7.5 mg, the dose of metformin is from about 100 mg to about 150 mg, and the dose of glimepiride is from about 0.2 mg to about 0.3 mg.

In some embodiments, the dose of each of (a) a DPP IV inhibitor; (b) a biguanide; and (c) a sulfonylurea is about 25% of the lowest diabetes therapeutic dose (LDTD) for each of (a), (b), and (c). In some embodiments, the dose of sitagliptin is about 6.25 mg, the dose of metformin is about 125 mg, and the dose of glimepiride is about 0.25 mg. In some embodiments, the dose of sitagliptin is about 6.25 mg, the dose of metformin hydrochloride is about 125 mg, and the dose of glimepiride is about 0.25 mg.

In some embodiments, the dose of each of the DPP IV inhibitor, the biguanide, and the sulfonylurea is from about 30% to about 40% of the lowest diabetes therapeutic dose.

In some embodiments, the dose of the DPP IV inhibitor is about 30%, about 31%, about 32%, about 33%, about 34%, about 35%, about 36%, about 37%, about 38%, about 39%, or about 40% of the lowest diabetes therapeutic dose. In some embodiments, the dose of the DPP IV inhibitor is about 33% of the lowest diabetes therapeutic dose. In some embodiments, the DPP IV inhibitor is sitagliptin. In some embodiments, the dose of sitagliptin is about 7.5, about 7.75, about 8.0, about 8.25, about 8.5, about 8.75, about 9.0, about 9.25, about 9.5, about 9.75, or about 10 mg.

In some embodiments, the dose of the biguanide inhibitor is about 30%, about 31%, about 32%, about 33%, about 34%, about 35%, about 36%, about 37%, about 38%, about 39%, or about 40% of the lowest diabetes therapeutic dose. In some embodiments, the dose of the biguanide is about 33% of the lowest diabetes therapeutic dose. In some embodiments, the biguanide is metformin or metformin hydrochloride. In some embodiments, the dose of metformin or metformin hydrochloride is about 150, about 155, about 160, about 165, about 170, about 175, about 180, about 185, about 190, about 195, or about 200 mg.

In some embodiments, the dose of the sulfonylurea is about 30%, about 31%, about 32%, about 33%, about 34%, about 35%, about 36%, about 37%, about 38%, about 39%, or about 40% of the lowest diabetes therapeutic dose. In some embodiments, the dose of the sulfonylurea is about 33% of the lowest diabetes therapeutic dose. In some embodiments, the sulfonylurea is glimepiride. In some embodiments, the dose of glimepiride is about 0.30, about 0.31, about 0.32, about 0.33, about 0.34, about 0.35, about 0.36, about 0.37, about 0.38, about 0.39, or about 0.40 mg.

In some embodiments, the lowest diabetes therapeutic dose (LDTD) and the corresponding proposed dose and proposed dose range for the following compounds are as described in Table 2.

TABLE 2

| | | | Proposed |
|---|---|---|---|
| | Lowest Diabetes | Proposed | Dose Range |
| | Therapeutic Dose | Dose (mg) | 30%-40% |
| Agent | (LDTD) (mg) | (33% LDTD) | LDTD (mg) |
| sitagliptin | 25 | 8.25 | 7.5-10 |
| vildagliptin | 50 | 16.5 | 15-20 |
| saxagliptin | 2.5 | 0.825 | 0.75-1 |
| linagliptin | 5 | 1.65 | 1.5-2 |
| alogliptin | 6.25 | 2.0625 | 1.875-2.5 |
| metformin | 500 | 165 | 150-200 |
| acetohexamide | 250 | 62.5 | 75-100 |
| tolazamide | 100 | 0.33 | 30-40 |
| tolbutamide | 250 | 82.5 | 75-100 |
| glibenclamide | 1.25 | 0.4125 | 0.375-0.5 |
| (glyburide) | | | |
| gliclazide | 30 | 9.9 | 9-12 |
| glipizide | 2.5 | 0.825 | 0.75-1 |
| gliquidone | 30 | 9.9 | 9-12 |
| glimepiride | 1 | 0.33 | 0.3-0.4 |

In some embodiments, the pharmaceutical composition comprises: (a) sitagliptin as a DPP W inhibitor; (b) metformin as a biguanide; and (c) glimepiride as a sulfonylurea. In some embodiments, the dose of sitagliptin is from about 7.5 mg to about 10 mg, the dose of metformin is from about 150 mg to about 200 mg, and the dose of glimepiride is from about 0.3 mg to about 0.4 mg.

In some embodiments, the dose of each of (a) a DPP IV inhibitor; (b) a biguanide; and (c) a sulfonylurea is about 33% of the lowest diabetes therapeutic dose (LDTD) for each of (a), (b), and (c). In some embodiments, the dose of sitagliptin is about 8.25 mg, the dose of metformin is about 165 mg, and the dose of glimepiride is about 0.33 mg. In some embodiments, the dose of sitagliptin is about 8.25 mg, the dose of metformin hydrochloride is about 165 mg, and the dose of glimepiride is about 0.33 mg.

In some embodiments, the dose of the DPP IV inhibitor is from about 40% to about 75% of the lowest diabetes therapeutic dose. In some embodiments, the dose of the DPP IV inhibitor is from about 40% to about 70% of the lowest diabetes therapeutic dose. In some embodiments, the dose of the DPP IV inhibitor is from about 40% to about 65% of the lowest diabetes therapeutic dose. In some embodiments, the dose of the DPP IV inhibitor is from about 40% to about 60% of the lowest diabetes therapeutic dose. In some embodiments, the dose of the DPP IV inhibitor is from about 40% to about 55% of the lowest diabetes therapeutic dose. In some embodiments, the dose of the DPP IV inhibitor is from about 40% to about 50% of the lowest diabetes therapeutic dose. In some embodiments, the dose of the DPP IV inhibitor is from about 40% to about 45% of the lowest diabetes therapeutic dose.

In some embodiments, the dose of the DPP IV inhibitor is from about 45% to about 75% of the lowest diabetes therapeutic dose. In some embodiments, the dose of the DPP IV inhibitor is from about 45% to about 70% of the lowest diabetes therapeutic dose. In some embodiments, the dose of the DPP IV inhibitor is from about 45% to about 65% of the lowest diabetes therapeutic dose. In some embodiments, the dose of the DPP IV inhibitor is from about 45% to about 60% of the lowest diabetes therapeutic dose. In some embodiments, the dose of the DPP IV inhibitor is from about 45% to about 55% of the lowest diabetes therapeutic dose. In some embodiments, the dose of the DPP IV inhibitor is from about 45% to about 50% of the lowest diabetes therapeutic dose.

In some embodiments, the dose of the DPP IV inhibitor is from about 50% to about 75% of the lowest diabetes therapeutic dose. In some embodiments, the dose of the DPP IV inhibitor is from about 50% to about 70% of the lowest diabetes therapeutic dose. In some embodiments, the dose of the DPP IV inhibitor is from about 50% to about 65% of the lowest diabetes therapeutic dose. In some embodiments, the dose of the DPP IV inhibitor is from about 50% to about 60% of the lowest diabetes therapeutic dose. In some embodiments, the dose of the DPP IV inhibitor is from about 50% to about 55% of the lowest diabetes therapeutic dose.

In some embodiments, the dose of the DPP IV inhibitor is from about 55% to about 75% of the lowest diabetes therapeutic dose. In some embodiments, the dose of the DPP IV inhibitor is from about 55% to about 70% of the lowest diabetes therapeutic dose. In some embodiments, the dose of the DPP IV inhibitor is from about 55% to about 65% of the lowest diabetes therapeutic dose. In some embodiments, the dose of the DPP IV inhibitor is from about 55% to about 60% of the lowest diabetes therapeutic dose.

In some embodiments, the dose of the DPP IV inhibitor is from about 60% to about 75% of the lowest diabetes therapeutic dose. In some embodiments, the dose of the DPP IV inhibitor is from about 60% to about 70% of the lowest diabetes therapeutic dose. In some embodiments, the dose of the DPP IV inhibitor is from about 60% to about 65% of the lowest diabetes therapeutic dose.

In some embodiments, the dose of the DPP IV inhibitor is from about 65% to about 75% of the lowest diabetes therapeutic dose. In some embodiments, the dose of the DPP IV inhibitor is from about 65% to about 70% of the lowest diabetes therapeutic dose. In some embodiments, the dose of the DPP IV inhibitor is from about 70% to about 75% of the lowest diabetes therapeutic dose.

In some embodiments, the dose of the biguanide is from about 40% to about 75% of the lowest diabetes therapeutic dose. In some embodiments, the dose of the biguanide is from about 40% to about 70% of the lowest diabetes therapeutic dose. In some embodiments, the dose of the biguanide is from about 40% to about 65% of the lowest diabetes therapeutic dose. In some embodiments, the dose of the biguanide is from about 40% to about 60% of the lowest diabetes therapeutic dose. In some embodiments, the dose of the biguanide is from about 40% to about 55% of the lowest diabetes therapeutic dose. In some embodiments, the dose of the biguanide is from about 40% to about 50% of the lowest diabetes therapeutic dose. In some embodiments, the dose of the biguanide is from about 40% to about 45% of the lowest diabetes therapeutic dose.

In some embodiments, the dose of the biguanide is from about 45% to about 75% of the lowest diabetes therapeutic dose. In some embodiments, the dose of the biguanide is from about 45% to about 70% of the lowest diabetes therapeutic dose. In some embodiments, the dose of the biguanide is from about 45% to about 65% of the lowest diabetes therapeutic dose. In some embodiments, the dose of the biguanide is from about 45% to about 60% of the lowest diabetes therapeutic dose. In some embodiments, the dose of the biguanide is from about 45% to about 55% of the lowest diabetes therapeutic dose. In some embodiments, the dose of the biguanide is from about 45% to about 50% of the lowest diabetes therapeutic dose.

In some embodiments, the dose of the biguanide is from about 50% to about 75% of the lowest diabetes therapeutic dose. In some embodiments, the dose of the biguanide is from about 50% to about 70% of the lowest diabetes therapeutic dose. In some embodiments, the dose of the biguanide is from about 50% to about 65% of the lowest diabetes therapeutic dose. In some embodiments, the dose of the biguanide is from about 50% to about 60% of the lowest diabetes therapeutic dose. In some embodiments, the dose of the biguanide is from about 50% to about 55% of the lowest diabetes therapeutic dose.

In some embodiments, the dose of the biguanide is from about 55% to about 75% of the lowest diabetes therapeutic dose. In some embodiments, the dose of the biguanide is from about 55% to about 70% of the lowest diabetes therapeutic dose. In some embodiments, the dose of the biguanide is from about 55% to about 65% of the lowest diabetes therapeutic dose. In some embodiments, the dose of the biguanide is from about 55% to about 60% of the lowest diabetes therapeutic dose.

In some embodiments, the dose of the biguanide is from about 60% to about 75% of the lowest diabetes therapeutic dose. In some embodiments, the dose of the biguanide is from about 60% to about 70% of the lowest diabetes therapeutic dose. In some embodiments, the dose of the biguanide is from about 60% to about 65% of the lowest diabetes therapeutic dose.

In some embodiments, the dose of the biguanide is from about 65% to about 75% of the lowest diabetes therapeutic dose. In some embodiments, the dose of the biguanide is from about 65% to about 70% of the lowest diabetes therapeutic dose. In some embodiments, the dose of the biguanide is from about 70% to about 75% of the lowest diabetes therapeutic dose.

In some embodiments, the dose of the sulfonylurea is from about 40% to about 75% of the lowest diabetes therapeutic dose. In some embodiments, the dose of the sulfonylurea is from about 40% to about 70% of the lowest diabetes therapeutic dose. In some embodiments, the dose of the sulfonylurea is from about 40% to about 65% of the lowest diabetes therapeutic dose. In some embodiments, the dose of the sulfonylurea is from about 40% to about 60% of the lowest diabetes therapeutic dose. In some embodiments, the dose of the sulfonylurea is from about 40% to about 55% of the lowest diabetes therapeutic dose. In some embodiments, the dose of the sulfonylurea is from about 40% to about 50% of the lowest diabetes therapeutic dose. In some embodiments, the dose of the sulfonylurea is from about 40% to about 45% of the lowest diabetes therapeutic dose.

In some embodiments, the dose of the sulfonylurea is from about 45% to about 75% of the lowest diabetes therapeutic dose. In some embodiments, the dose of the sulfonylurea is from about 45% to about 70% of the lowest diabetes therapeutic dose. In some embodiments, the dose of the sulfonylurea is from about 45% to about 65% of the lowest diabetes therapeutic dose. In some embodiments, the dose of the sulfonylurea is from about 45% to about 60% of the lowest diabetes therapeutic dose. In some embodiments, the dose of the sulfonylurea is from about 45% to about 55% of the lowest diabetes therapeutic dose. In some embodiments, the dose of the sulfonylurea is from about 45% to about 50% of the lowest diabetes therapeutic dose.

In some embodiments, the dose of the sulfonylurea is from about 50% to about 75% of the lowest diabetes therapeutic dose. In some embodiments, the dose of the sulfonylurea is from about 50% to about 70% of the lowest diabetes therapeutic dose. In some embodiments, the dose of the sulfonylurea is from about 50% to about 65% of the lowest diabetes therapeutic dose. In some embodiments, the dose of the sulfonylurea is from about 50% to about 60% of the lowest diabetes therapeutic dose. In some embodiments, the dose of the sulfonylurea is from about 50% to about 55% of the lowest diabetes therapeutic dose.

In some embodiments, the dose of the sulfonylurea is from about 55% to about 75% of the lowest diabetes therapeutic dose. In some embodiments, the dose of the sulfonylurea is from about 55% to about 70% of the lowest diabetes therapeutic dose. In some embodiments, the dose of the sulfonylurea is from about 55% to about 65% of the lowest diabetes therapeutic dose. In some embodiments, the dose of the sulfonylurea is from about 55% to about 60% of the lowest diabetes therapeutic dose.

In some embodiments, the dose of the sulfonylurea is from about 60% to about 75% of the lowest diabetes therapeutic dose. In some embodiments, the dose of the sulfonylurea is from about 60% to about 70% of the lowest diabetes therapeutic dose. In some embodiments, the dose of the sulfonylurea is from about 60% to about 65% of the lowest diabetes therapeutic dose.

In some embodiments, the dose of the sulfonylurea is from about 65% to about 75% of the lowest diabetes therapeutic dose. In some embodiments, the dose of the sulfonylurea is from about 65% to about 70% of the lowest diabetes therapeutic dose. In some embodiments, the dose of the sulfonylurea is from about 70% to about 75% of the lowest diabetes therapeutic dose.

In some embodiments, the dose of the sulfonylurea is from about 40% to about 75% of the lowest diabetes therapeutic dose. In some embodiments, the dose of the sulfonylurea is from about 40% to about 70% of the lowest diabetes therapeutic dose. In some embodiments, the dose of the sulfonylurea is from about 40% to about 65% of the lowest diabetes therapeutic dose. In some embodiments, the dose of the sulfonylurea is from about 40% to about 60% of the lowest diabetes therapeutic dose. In some embodiments, the dose of the sulfonylurea is from about 40% to about 55% of the lowest diabetes therapeutic dose. In some embodiments, the dose of the sulfonylurea is from about 40% to about 50% of the lowest diabetes therapeutic dose. In some embodiments, the dose of the sulfonylurea is from about 40% to about 45% of the lowest diabetes therapeutic dose.

In some embodiments, the dose of the sulfonylurea is from about 45% to about 75% of the lowest diabetes therapeutic dose. In some embodiments, the dose of the sulfonylurea is from about 45% to about 70% of the lowest diabetes therapeutic dose. In some embodiments, the dose of the sulfonylurea is from about 45% to about 65% of the lowest diabetes therapeutic dose. In some embodiments, the dose of the sulfonylurea is from about 45% to about 60% of the lowest diabetes therapeutic dose. In some embodiments, the dose of the sulfonylurea is from about 45% to about 55% of the lowest diabetes therapeutic dose. In some embodiments, the dose of the sulfonylurea is from about 45% to about 50% of the lowest diabetes therapeutic dose.

In some embodiments, the dose of the sulfonylurea is from about 50% to about 75% of the lowest diabetes therapeutic dose. In some embodiments, the dose of the sulfonylurea is from about 50% to about 70% of the lowest diabetes therapeutic dose. In some embodiments, the dose of the sulfonylurea is from about 50% to about 65% of the lowest diabetes therapeutic dose. In some embodiments, the dose of the sulfonylurea is from about 50% to about 60% of the lowest diabetes therapeutic dose. In some embodiments, the dose of the sulfonylurea is from about 50% to about 55% of the lowest diabetes therapeutic dose.

In some embodiments, the dose of the sulfonylurea is from about 55% to about 75% of the lowest diabetes therapeutic dose. In some embodiments, the dose of the sulfonylurea is from about 55% to about 70% of the lowest diabetes therapeutic dose. In some embodiments, the dose of the sulfonylurea is from about 55% to about 65% of the lowest diabetes therapeutic dose. In some embodiments, the dose of the sulfonylurea is from about 55% to about 60% of the lowest diabetes therapeutic dose.

In some embodiments, the dose of the sulfonylurea is from about 60% to about 75% of the lowest diabetes therapeutic dose. In some embodiments, the dose of the sulfonylurea is from about 60% to about 70% of the lowest diabetes therapeutic dose. In some embodiments, the dose of the sulfonylurea is from about 60% to about 65% of the lowest diabetes therapeutic dose.

In some embodiments, the dose of the sulfonylurea is from about 65% to about 75% of the lowest diabetes therapeutic dose. In some embodiments, the dose of the sulfonylurea is from about 65% to about 70% of the lowest diabetes therapeutic dose. In some embodiments, the dose of the sulfonylurea is from about 70% to about 75% of the lowest diabetes therapeutic dose.

In some embodiments, the dose of each of the DPP IV inhibitor, the biguanide, and the sulfonylurea is from about 40% to about 60% of the lowest diabetes therapeutic dose.

In some embodiments, the dose of each of the DPP IV inhibitor, the biguanide, and the sulfonylurea is from about 40% to about 50% of the lowest diabetes therapeutic dose.

In some embodiments, the dose of each of the DPP IV inhibitor, the biguanide, and the sulfonylurea is from about 50% to about 60% of the lowest diabetes therapeutic dose.

In some embodiments, the dose of each of the DPP IV inhibitor, the biguanide, and the sulfonylurea is from about 45% to about 55% of the lowest diabetes therapeutic dose.

In some embodiments, the dose of the DPP IV inhibitor is about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60% of the lowest diabetes therapeutic dose. In some embodiments, the dose of the DPP IV inhibitor is about 50% of the lowest diabetes therapeutic dose. In some embodiments, the DPP IV inhibitor is sitagliptin. In some embodiments, the dose of sitagliptin is about 10, about 10.25, about 10.5, about 10.75, about 11, about 11.25, about 11.5, about 11.75, about 12, about 12.25, about 12.5, about 12.75, about 13, about 13.25, about 13.5, about 13.75, about 14, about 14.25, about 14.5, about 14.75, or about 15 mg.

In some embodiments, the dose of the biguanide inhibitor is about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60% of the lowest diabetes therapeutic dose. In some embodiments, the dose of the biguanide is about 50% of the lowest diabetes therapeutic dose. In some embodiments, the biguanide is metformin or metformin hydrochloride. In some embodiments, the dose of metformin or metformin hydrochloride is about 200, about 205, about 210, about 215, about 220, about 225, about 230, about 235, about 240, about 245, about 250, about 255, about 260, about 265, about 270, about 275, about 280, about 285, about 290, about 295, or about 300 mg.

In some embodiments, the dose of the sulfonylurea is about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60% of the lowest diabetes therapeutic dose. In some embodiments, the dose of the sulfonylurea is about 50% of the lowest diabetes therapeutic dose. In some embodiments, the sulfonylurea is glimepiride. In some embodiments, the dose of glimepiride is about 0.40, about 0.40, about 0.41, about 0.42, about 0.43, about 0.44, about 0.45, about 0.46, about 0.47, about 0.48, about 0.49, about 0.50, about 0.51, about 0.52, about 0.53, about 0.54, about 0.55, about 0.56, about 0.57, about 0.58, about 0.59, or about 0.60 mg.

In some embodiments, the lowest diabetes therapeutic dose (LDTD) and the corresponding proposed dose and proposed dose range for the following compounds are as described in Table 3.

TABLE 3

| | Lowest | | | |
|---|---|---|---|---|
| | Diabetes | | Proposed | Proposed |
| | Therapeutic | Proposed | Dose Range | Dose Range |
| | Dose | Dose (mg) | 40%-60% | 45%-55% |
| | (LDTD) | (50% | LDTD | LDTD |
| Agent | (mg) | LDTD) | (mg) | (mg) |
| sitagliptin | 25 | 12.5 | 10-15 | 11.25-12.5 |
| vildagliptin | 50 | 25 | 20-30 | 22.5-27.5 |
| saxagliptin | 2.5 | 1.25 | 1-1.5 | 1.125-1.375 |
| linagliptin | 5 | 2.5 | 2-3 | 2.25-2.75 |
| alogliptin | 6.25 | 3.125 | 2.5-3.75 | 2.8125-3.4375 |
| metformin | 500 | 250 | 200-300 | 225-275 |
| acetohexamide | 250 | 125 | 100-150 | 112.5-137.5 |
| tolazamide | 100 | 50 | 40-60 | 45-55 |
| tolbutamide | 250 | 125 | 100-150 | 112.5-137.5 |
| glibenclamide | 1.25 | 0.625 | 0.5-0.75 | 0.5625-0.6875 |
| (glyburide) | | | | |
| gliclazide | 30 | 15 | 12-18 | 13.5-16.5 |
| glipizide | 2.5 | 1.25 | 1-1.5 | 1.125-1.375 |
| gliquidone | 30 | 15 | 12-18 | 13.5-16.5 |
| glimepiride | 1 | 0.5 | 0.4-0.6 | 0.45-55 |

In some embodiments, the pharmaceutical composition comprises: (a) sitagliptin as a DPP IV inhibitor; (b) metformin as a biguanide; and (c) glimepiride as a sulfonylurea. In some embodiments, the dose of sitagliptin is from about 10 mg to about 15 mg, the dose of metformin is from about 200 mg to about 300 mg, and the dose of glimepiride is from about 0.4 mg to about 0.6 mg.

In some embodiments, the dose of sitagliptin is from about 11.25 mg to about 12.5 mg, the dose of metformin is from about 225 mg to about 275 mg, and the dose of glimepiride is from about 0.45 mg to about 0.55 mg. In some embodiments, metformin is metformin hydrochloride.

In some embodiments, the dose of each of (a) a DPP IV inhibitor; (b) a biguanide; and (c) a sulfonylurea is about 50% of the lowest diabetes therapeutic dose (LDTD) for each of (a), (b), and (c). In some embodiments, the dose of sitagliptin is about 12.5 mg, the dose of metformin is about 250 mg, and the dose of glimepiride is about 0.5 mg. In some embodiments, the dose of sitagliptin is about 12.5 mg, the dose of metformin hydrochloride is about 250 mg, and the dose of glimepiride is about 0.5 mg.

In some embodiments, the dose of the DPP IV inhibitor is from about 60% to about 75% of the lowest diabetes therapeutic dose.

In some embodiments, the dose of the DPP IV inhibitor is about 60%, about 61%, about 62%, about 63%, about 64%, about 65%, about 66%, about 67%, about 68%, about 69%, about 70%, about 71%, about 72%, about 73%, about 74%, or about 75% of the lowest diabetes therapeutic dose. In some embodiments, the dose of the DPP IV inhibitor is about 70% of the lowest diabetes therapeutic dose. In some embodiments, the DPP IV inhibitor is sitagliptin. In some embodiments, the dose of sitagliptin is about 15, about 15.25, about 15.5, about 15.75, about 16, about 16.25, about 16.5, about 16.75, about 17, about 17.25, about 17.5, about 17.75, about 18, about 18.25, about 18.5, or about 18.75 mg.

In some embodiments, the dose of the biguanide is from about 60% to about 75% of the lowest diabetes therapeutic dose. In some embodiments, the dose of the biguanide inhibitor is about 60%, about 61%, about 62%, about 63%, about 64%, about 65%, about 66%, about 67%, about 68%, about 69%, about 70%, about 71%, about 72%, about 73%, about 74%, or about 75% of the lowest diabetes therapeutic dose. In some embodiments, the dose of the biguanide is about 70% of the lowest diabetes therapeutic dose. In some embodiments, the biguanide is metformin or metformin hydrochloride. In some embodiments, the dose of metformin or metformin hydrochloride is about 300, about 305, about 310, about 315, about 320, about 325, about 330, about 335, about 340, about 345, about 350, about 355, about 360, about 365, about 370, or about 375 mg.

In some embodiments, the dose of the sulfonylurea is from about 60% to about 75% of the lowest diabetes therapeutic dose.

In some embodiments, the dose of the sulfonylurea is about 60%, about 61%, about 62%, about 63%, about 64%, about 65%, about 66%, about 67%, about 68%, about 69%, about 70%, about 71%, about 72%, about 73%, about 74%, or about 75% of the lowest diabetes therapeutic dose. In some embodiments, the dose of the sulfonylurea is about 70% of the lowest diabetes therapeutic dose. In some embodiments, the sulfonylurea is glimepiride. In some embodiments, the dose of glimepiride is about 0.60, about 0.60, about 0.61, about 0.62, about 0.63, about 0.64, about 0.65, about 0.66, about 0.67, about 0.68, about 0.69, about 0.70, about 0.71, about 0.72, about 0.73, about 0.74, or about 0.75 mg.

In some embodiments, the lowest diabetes therapeutic dose (LDTD) and the corresponding proposed dose and proposed dose range for the following compounds are as described in Table 4.

TABLE 4

| | Lowest | | | |
|---|---|---|---|---|
| | Diabetes | | Proposed | Proposed |
| | Therapeutic | Proposed | Dose Range | Dose Range |
| | Dose | Dose (mg) | 60%-75% | 60%-70% |
| | (LDTD) | 70% | LDTD | LDTD |
| Agent | (mg) | LDTD | (mg) | (mg) |
| sitagliptin | 25 | 17.5 | 15-18.75 | 15-17.5 |
| vildagliptin | 50 | 35 | 30-37.5 | 30-35 |
| saxagliptin | 2.5 | 1.75 | 1.5-1.875 | 1.5-1.75 |
| linagliptin | 5 | 3.5 | 3-3.75 | 3-3.5 |
| alogliptin | 6.25 | 4.375 | 3.75-4.6875 | 3.75-4.375 |
| metformin | 500 | 350 | 300-375 | 300-350 |
| acetohexamide | 250 | 175 | 150-187.5 | 150-175 |
| tolazamide | 100 | 70 | 60-75 | 60-70 |
| tolbutamide | 250 | 175 | 150-187.5 | 150-175 |
| glibenclamide | 1.25 | 0.875 | 0.75-0.9375 | 0.75-0.875 |
| (glyburide) | | | | |
| gliclazide | 30 | 21 | 18-22.5 | 18-21 |
| glipizide | 2.5 | 1.75 | 1.5-1.875 | 1.5-1.75 |
| gliquidone | 30 | 21 | 18-22.5 | 18-21 |
| glimepiride | 1 | 0.7 | 0.6-0.75 | 0.6-0.70 |

In some embodiments, the pharmaceutical composition comprises: (a) sitagliptin as a DPP IV inhibitor; (b) metformin as a biguanide; and (c) glimepiride as a sulfonylurea. In some embodiments, the dose of sitagliptin is from about 15 mg to about 18.75 mg, the dose of metformin hydrochloride is from about 300 mg to about 375 mg, and the dose of glimepiride is from about 0.6 mg to about 0.75 mg. In some embodiments, the dose of sitagliptin is from about 15 mg to about 17.5 mg, the dose of metformin hydrochloride is from about 300 mg to about 350 mg, and the dose of glimepiride is from about 0.6 mg to about 0.70 mg. In some embodiments, metformin is metformin hydrochloride.

In some embodiments, the dose of (a) a DPP IV inhibitor; (b) a biguanide; and (c) a sulfonylurea is about 70% of the lowest diabetes therapeutic dose (LDTD) for each of (a), (b), and (c). In some embodiments, the dose of sitagliptin is about 17.5 mg, the dose of metformin is about 350 mg, and the dose of glimepiride is about 0.7 mg. In some embodiments, the dose of sitagliptin is about 17.5 mg, the dose of metformin hydrochloride is about 350 mg, and the dose of glimepiride is about 0.7 mg.

In some embodiments, the dose of (a) a DPP IV inhibitor; (b) a biguanide; and (c) a sulfonylurea is about 70% of the lowest diabetes therapeutic dose (LDTD) for each of (a) and (b), and about 50% of the lowest diabetes therapeutic dose (LDTD) for (c).

In some embodiments, the lowest diabetes therapeutic dose (LDTD) and the corresponding proposed dose and proposed dose range for the following compounds are as described in Table 5.

TABLE 5

| | Lowest | | | | |
|---|---|---|---|---|---|
| | Diabetes | | | Proposed | |
| | Therapeutic | Proposed | | Dose Range | |
| | Dose (LDTD) | Dose (mg) | | (% LDTD) | |
| Agent | (mg) | (% LDTD) | | (mg) | |
| sitagliptin | 25 | 17.5 | (70) | 16.25-18.75 | (65-75) |
| vildagliptin | 50 | 35 | (70) | 32.5-37.5 | (65-75) |
| saxagliptin | 2.5 | 1.75 | (70) | 1.625-1.875 | (65-75) |
| linagliptin | 5.0 | 3.5 | (70) | 3.25-3.75 | (65-75) |
| alogliptin | 6.25 | 4.375 | (70) | 4.0625-4.6875 | (65-75) |
| metformin | 500 | 350 | (70) | 325-375 | (65-75) |
| acetohexamide | 250 | 125 | (50) | (45-55) | |
| tolazamide | 100 | 50 | (50) | 45-55 | (45-55) |
| tolbutamide | 250 | 125 | (50) | 112.5-137.5 | (45-55) |
| glibenclamide | 1.25 | 0.625 | (50) | 0.5625-0.6875 | (45-55) |
| (glyburide) | | | | | |
| gliclazide | 30 | 15 | (50) | 13.5-16.5 | (45-55) |
| glipizide | 2.5 | 1.25 | (50) | 1.125-1.375 | (45-55) |
| gliquidone | 30 | 15 | (50) | 13.5-16.5 | (45-55) |
| glimepiride | 1 | 0.5 | (50) | 0.45-0.55 | (45-55) |

In some embodiments, the pharmaceutical composition comprises: (a) sitagliptin as a DPP W inhibitor; (b) metformin as a biguanide; and (c) glimepiride as a sulfonylurea. In some embodiments, the dose of sitagliptin is about 17.5 mg, the dose of metformin is about 350 mg, and the dose of glimepiride is about 0.5 mg. In some embodiments, the dose of sitagliptin is about 17.5 mg, the dose of metformin hydrochloride is about 350 mg, and the dose of glimepiride is about 0.5 mg.

### Formulations

In some embodiments, the DPP IV inhibitor, biguanide, and sulfonylurea are provided in one formulation. In some embodiments, the DPP IV inhibitor, biguanide, and sulfonylurea are each provided in a separate formulation. In some embodiments, two of the DPP IV inhibitor, biguanide, and sulfonylurea are provided in one formulation. In some embodiments, the DPP IV inhibitor and biguanide are provided in one formulation. In some embodiments, the DPP IV inhibitor and sulfonylurea are provided in one formulation. In some embodiments, the biguanide and sulfonylurea are provided in one formulation. In some embodiments, the pharmaceutical composition is in the form of pill, tablet, or capsule. In some embodiments, the pharmaceutical composition is in the form of pill. In some embodiments, the pharmaceutical composition is in the form of tablet. In some embodiments, the pharmaceutical composition is in the form of capsule. In some embodiments, the pharmaceutical composition is suitable for oral administration.

Other suitable formulations include, but are not limited to, those suitable for rectal, topical, buccal, parenteral (e.g., subcutaneous, intramuscular, intradermal, or intravenous) rectal, vaginal, or aerosol administration, although the most suitable form of administration in any given case will depend on the degree and severity of the condition being treated and on the nature of the particular compound being used. For example, disclosed compositions are formulated as a unit dose.

Exemplary pharmaceutical compositions used in the form of a pharmaceutical preparation, for example, in solid, semisolid, or liquid form, which includes one or more of a disclosed compound, as an active ingredient, in admixture with an organic or inorganic carrier or excipient suitable for external, enteral, or parenteral applications. The active ingredient, in some embodiments, are compounded, for example, with the usual non-toxic, pharmaceutically acceptable carriers for tablets, pellets, capsules, suppositories, solutions, emulsions, suspensions, and any other form suitable for use. The active object compound is included in the pharmaceutical composition in an amount sufficient to produce the desired effect upon the process or condition of the disease.

For preparing solid compositions such as tablets, the principal active ingredient, in some embodiments, are mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a disclosed compound or a non-toxic pharmaceutically acceptable salt thereof. In some embodiments, when referring to these preformulation compositions as homogeneous, it is meant that the active ingredient is dispersed evenly throughout the composition so that the composition are readily subdivided into equally effective unit dosage forms such as tablets, pills, and capsules.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions, in some embodiments, also comprise buffering agents. Solid compositions of a similar type, in some embodiments, are also employed as fillers in soft and hard-filled gelatin capsules using such excipients as lactose or milk sugars, as well as high molecular weight polyethylene glycols and the like.

In some embodiments, a tablet is made by compression or molding, optionally with one or more accessory ingredients. Compressed tablets, in some embodiments, are prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets, in some embodiments, are made by molding in a suitable machine a mixture of the subject composition moistened with an inert liquid diluent. In some embodiments, capsules are prepared by encapsulating tablets in hard-gelatin capsules (e.g., over-encapsulation). Tablets, and other solid dosage forms, such as dragees, capsules, pills and granules, in some embodiments, are optionally scored or prepared with coatings and shells, such as enteric coatings and other coatings well known in the pharmaceutical-formulating art.

### Methods of Treatment

The pharmaceutical compositions described herein are useful for treating a metabolic disorder in a subject in need thereof. The pharmaceutical compositions described herein are useful for treating diabetes in a subject in need thereof.

The high incidence of therapeutic failure is a major contributor to the high rate of long-term hyperglycemia-associated complications or chronic damages (including microvascular complications such as diabetic nephropathy, retinopathy or neuropathy, and macrovascular complications such as coronary heart disease, cerebrovascular disease, and peripheral vascular disease) in patients with type 2 diabetes. Therefore, there is an unmet medical need for methods, medicaments, and pharmaceutical compositions with a good efficacy with regard to glycemic control, with regard to disease-modifying properties and with regard to reduction of cardiovascular morbidity and mortality while at the same time showing an improved safety profile.

In some embodiments, the treatment or methods of the present disclosure result in one or more of the following:
i. preventing, slowing progression of, delaying, or treating a metabolic disorder;
ii. preventing, slowing progression of, delaying, or treating diabetes;
iii. improving glycemic control and/or for reducing of fasting plasma glucose, of postprandial plasma glucose, of continuously measured blood glucose, and/or of glycosylated hemoglobin HbAlc;
iv. preventing, slowing, delaying or reversing progression from impaired glucose tolerance, impaired fasting blood glucose, and/or insulin resistance from metabolic syndrome and/or type 2 diabetes mellitus;
v. preventing, slowing progression of, delaying or treating of a condition or disorder selected from the group consisting of vascular and non-vascular complications of diabetes mellitus;
vi. preventing, slowing progression of, delaying, or treating impairment of renal function;
vii. preventing, slowing progression of, delaying, or treating retinal vascular disease;
viii. reducing body weight and/or body fat or preventing an increase in body weight and/or body fat or facilitating a reduction in body weight and/or body fat;
ix. preventing or treating the degeneration of pancreatic beta cells and/or for improving and/or restoring the functionality of pancreatic beta cells and/or restoring the functionality of pancreatic insulin secretion;
x. preventing, slowing, delaying or treating diseases or conditions attributed to an abnormal accumulation of ectopic fat;
xi. maintaining and/or improving the insulin sensitivity and/or for treating or preventing hyperinsulinemia and/or insulin resistance,
xii. preventing, slowing progression of, delaying, or treating new onset diabetes after transplantation (NODAT) and/or post-transplant metabolic syndrome (PTMS);
xiii. preventing, delaying, or reducing NODAT and/or PTMS associated complications including microvascular and macrovascular diseases and events, graft rejection, infection, and death;
xiv. treating hyperuricemia and hyperuricemia associated conditions;
xv. treating or preventing kidney stones; and/or
xvi. treating hyponatremia.

In some embodiments, the treatment results in slowing progression of, delaying or treating a metabolic disorder, in particular of type 2 diabetes mellitus.

In some embodiments, the treatment results in an improvement in glycemic control in a patient in need thereof, in particular in patients with type 2 diabetes mellitus.

In some embodiments, the treatment results in an improvement in glycemic control in a patient with insufficient glycemic control despite monotherapy with an antidiabetic drug or despite combination therapy with two antidiabetic drugs.

In some embodiments, the treatment results in glucose lowering effects, effects on insulin levels, or combinations thereof. In some embodiments, the treatment results in glucose lowering effects, effects on insulin levels, or combinations thereof without any adverse events or low incidence of adverse evidence.

In some embodiments, the treatment results in glucose lowering effects at about 0.5 hour, about 1 hour, about 1.5 hours, about 2 hours, about 2.5 hours, about 3 hours, about 3.5 hours, about 4 hours, about 4.5 hours, about 5 hours, about 5.5 hours, about 6 hours, or more than about 6 hours following treatment. In some embodiments, the glucose lowering effects is determined by measuring primary endpoints, secondary endpoints, tertiary endpoints, or combinations thereof.

In some embodiments, the primary and secondary endpoints are the mean absolute change in plasma glucose and serum insulin, respectively at a certain time post-prandial from pre-prandial following the administration of a single dose of the treatment. In some embodiments, the primary and secondary endpoints are the mean absolute change in plasma glucose and serum insulin, respectively at about 0.5 hour, about 1 hour, about 1.5 hours, about 2 hours, about 2.5 hours, about 3 hours, about 3.5 hours, about 4 hours, about 4.5 hours, about 5 hours, about 5.5 hours, about 6 hours, or more than about 6 hours post-prandial from pre-prandial following the administration of a single dose of the treatment. In some embodiments, the primary and secondary endpoints are the mean absolute change in plasma glucose and serum insulin, respectively at about 2 hours post-prandial from pre-prandial following the administration of a single dose of the treatment. In some embodiments, the primary endpoint and secondary endpoint are compared to plasma glucose, serum insulin, or combinations thereof pre-prandial.

In some embodiments, the tertiary endpoints are determined as the area under the concentration time curve (AUC) of plasma glucose, serum insulin, or combinations thereof post-dose. In some embodiments, the tertiary endpoints are determined as the area under the concentration time curve (AUC) of plasma glucose, serum insulin, or combinations thereof post-meal. In some embodiments, the tertiary endpoints are determined as about 0.5 hour, about 1 hour, about 1.5 hours, about 2 hours, about 2.5 hours, about 3 hours, about 3.5 hours, about 4 hours, about 4.5 hours, about 5 hours, about 5.5 hours, about 6 hours, about 6.5 hours, about 7 hours, about 7.5 hours, about 8 hours, or more than about 8 hours of the AUC of plasma glucose, serum insulin, or combinations thereof post-dose. In some embodiments, the tertiary endpoints are determined as about 0.5 hour, about 1 hour, about 1.5 hours, about 2 hours, about 2.5 hours, about 3 hours, about 3.5 hours, about 4 hours, about 4.5 hours, about 5 hours, about 5.5 hours, about 6 hours, about 6.5 hours, about 7 hours, about 7.5 hours, about 8 hours, or more than about 8 hours of the AUC of plasma glucose, serum insulin, or combinations thereof post-meal. In some embodiments, the tertiary endpoints are compared to plasma glucose, serum insulin, or combinations thereof pre-prandial.

In some embodiments, treatment using compositions described herein (e.g., Composition A) results in a glucose lowering effect. In some embodiments, treatment using compositions described herein (e.g., Composition A) results in a glucose lowering effect by at least or about 5%, 10%, 15%, 20%, 30%, 40%, 50%, 60%, 70%, 80%, 90%, 95%, or more than 95% as compared to no treatment, placebo treatment, or treatment with one or two active agents of the composition. In some embodiments, the treatment results in post-prandial plasma glucose being lowered by at least or about 5%, 10%, 15%, 20%, 30%, 40%, 50%, 60%, 70%, 80%, 90%, 95%, or more than 95% as compared to no treatment, placebo treatment, or treatment with one or two active agents of the composition. In some embodiments, the treatment results in the about 0.5 hour, about 1 hour, about 1.5 hours, about 2 hours, about 2.5 hours, about 3 hours, about 3.5 hours, about 4 hours, about 4.5 hours, about 5 hours, about 5.5 hours, about 6 hours, about 6.5 hours, about 7 hours, about 7.5 hours, about 8 hours, or more than about 8 hours post-prandial plasma glucose being lowered at least or about 5%, 10%, 15%, 20%, 30%, 40%, 50%, 60%, 70%, 80%, 90%, 95%, or more than 95% as compared to no treatment, placebo treatment, or treatment with one or two active agents of the composition. In some embodiments, the treatment results in the about 0.5 hour to about 6 hours, about 1 hour to about 5 hours, or about 2 hours to about 4 hours post-prandial plasma glucose being lowered by at least or about 5%, 10%, 15%, 20%, 30%, 40%, 50%, 60%, 70%, 80%, 90%, 95%, or more than 95% as compared to no treatment, placebo treatment, or treatment with one or two active agents of the composition. In some embodiments, the glucose lowering effects of the treatment is compared pre-prandial.

In some embodiments, treatment using compositions described herein (e.g., Composition A) results in increased insulin levels. In some embodiments, the treatment using compositions described herein (e.g., Composition A) results in increased insulin levels by at least or about 5%, 10%, 15%, 20%, 30%, 40%, 50%, 60%, 70%, 80%, 90%, 95%, or more than 95% as compared to no treatment, placebo treatment, or treatment with one or two active agents of the composition. In some embodiments, the treatment results in post-prandial insulin levels being increased by at least or about 5%, 10%, 15%, 20%, 30%, 40%, 50%, 60%, 70%, 80%, 90%, 95%, or more than 95% as compared to no treatment, placebo treatment, or treatment with one or two active agents of the composition. In some embodiments, the treatment results in the about 0.5 hour, about 1 hour, about 1.5 hours, about 2 hours, about 2.5 hours, about 3 hours, about 3.5 hours, about 4 hours, about 4.5 hours, about 5 hours, about 5.5 hours, about 6 hours, about 6.5 hours, about 7 hours, about 7.5 hours, about 8 hours, or more than about 8 hours post-prandial insulin levels being increased by at least or about 5%, 10%, 15%, 20%, 30%, 40%, 50%, 60%, 70%, 80%, 90%, 95%, or more than 95% as compared to no treatment, placebo treatment, or treatment with one or two active agents of the composition. In some embodiments, the treatment results in the about 0.5 hour to about 6 hours, about 1 hour to about 5 hours, or about 2 hours to about 4 hours post-prandial insulin levels being increased by at least or about 5%, 10%, 15%, 20%, 30%, 40%, 50%, 60%, 70%, 80%, 90%, 95%, or more than 95% as compared to no treatment, placebo treatment, or treatment with one or two active agents of the composition. In some embodiments, the increased insulin levels of the treatment is compared pre-prandial.

In some embodiments, the treatment using compositions described herein (e.g., Composition A) results in plasma glucose levels, insulin levels, or combinations thereof returning to pre-prandial levels at a faster rate as compared to no treatment, placebo treatment, or treatment with one or two active agents of the composition. In some embodiments, the treatment using compositions described herein (e.g., Composition A) results in plasma glucose levels, insulin levels, or combinations thereof returning to pre-prandial levels at least or about 5%, 10%, 15%, 20%, 30%, 40%, 50%, 60%, 70%, 80%, 90%, 95%, or more than 95% faster as compared to no treatment, placebo treatment, or treatment with one or two active agents of the composition.

In some embodiments, the treatment results in slowing or delaying progression from impaired glucose tolerance (IGT), impaired fasting blood glucose (IFG), insulin resistance and/or metabolic syndrome to type 2 diabetes mellitus.

In some embodiments, the method results in prevention, slowing progression of, delaying or treatment of a condition or disorder from the group consisting of complications of diabetes mellitus.

In some embodiments, the treatment results in a reduction in the weight or prevention of an increase of the weight in a patient in need thereof.

In some embodiments, the method results in efficacious treatment of metabolic disorders, such as diabetes mellitus, impaired glucose tolerance (IGT), impaired fasting blood glucose (IFG), and/or hyperglycemia, with good pharmacological and/or pharmacokinetic and/or physicochemical properties.

In some embodiments, the method results in efficacious treatment of metabolic disorders, such as diabetes mellitus, impaired glucose tolerance (IGT), impaired fasting blood glucose (IFG), and/or hyperglycemia, with greater long term tolerability and reduced risk of side effects (e.g., low blood sugar, pancreatic cancer, hypersensitivity reactions including anaphylaxis, angioedema, rash, urticaria, cutaneous vasculitis, and exfoliative skin conditions including Stevens-Johnson syndrome; hepatic enzyme elevations; acute pancreatitis, including fatal and nonfatal hemorrhagic and necrotizing pancreatitis; worsening renal function, including acute renal failure (sometimes requiring dialysis); severe and disabling arthralgia; constipation; vomiting; headache; myalgia; pain in extremity; back pain; pruritus; and/or pemphigoid, joint pain, lactic acidosis, vitamin B12 and folic acid deficiency, nasopharyngitis, upper respiratory tract infection).

In some embodiments, treatment results in improved treatment of diabetes that is greater than the treatment obtained with the full lowest diabetic therapeutic dose of any one of the DPP IV inhibitor, the biguanide, and the sulfonylurea in the pharmaceutical composition.

In some embodiments, treatment results in improved treatment of diabetes that is greater than the treatment obtained with the full lowest diabetic therapeutic dose of the DPP IV inhibitor in the pharmaceutical composition. In some embodiments, treatment results in improved treatment of diabetes that is greater than the treatment obtained with the full lowest diabetic therapeutic dose of the biguanide in the pharmaceutical composition. In some embodiments, treatment results in improved treatment of diabetes that is greater than the treatment obtained with the full lowest diabetic therapeutic dose of the sulfonylurea in the pharmaceutical composition.

In some embodiments, treatment results in greater long term tolerability and reduced risk of side effects when compared to treatment with the full lowest diabetic therapeutic dose of any one of the DPP IV inhibitor, the biguanide, and the sulfonylurea in the pharmaceutical composition. In some embodiments, the treatment results in greater long term tolerability and reduced risk of side effects when compared to treatment with the full lowest diabetic therapeutic dose of the DPP IV inhibitor in the pharmaceutical composition. In some embodiments, the treatment results in greater long term tolerability and reduced risk of side effects when compared to treatment with the full lowest diabetic therapeutic dose of the biguanide in the pharmaceutical composition. In some embodiments, the treatment results in greater long term tolerability and reduced risk of side effects when compared to treatment with the full lowest diabetic therapeutic dose of the sulfonylurea in the pharmaceutical composition.

In some embodiments, treatment results in an improvement in diabetes and/or associated conditions that is greater than or equal to the improvement obtained with the combination of any two of the DPP IV inhibitor, the biguanide, and the sulfonylurea in the pharmaceutical composition. In some embodiments, treatment results in an improvement in diabetes and/or associated conditions that is greater than or equal to the improvement obtained with a combination of any two of the DPP IV inhibitor, the biguanide, and the sulfonylurea in the pharmaceutical composition, wherein the dose of each the DPP IV inhibitor, the biguanide, and the sulfonylurea is about 25% of the lowest diabetic therapeutic dose. In some embodiments, treatment results in an improvement in diabetes and/or associated conditions that is greater than or equal to the improvement obtained with a combination of any two of the DPP IV inhibitor, the biguanide, and the sulfonylurea in the pharmaceutical composition, wherein the dose of each the DPP IV inhibitor, the biguanide, and the sulfonylurea is about 33% of the lowest diabetic therapeutic dose. In some embodiments, treatment results in an improvement in diabetes and/or associated conditions that is greater than or equal to the improvement obtained with a combination of any two of the DPP IV inhibitor, the biguanide, and the sulfonylurea in the pharmaceutical composition, wherein the dose of each the DPP IV inhibitor, the biguanide, and the sulfonylurea is about 50% of the lowest diabetic therapeutic dose. In some embodiments, treatment results in an improvement in diabetes and/or associated conditions that is greater than or equal to the improvement obtained with a combination of any two of the DPP IV inhibitor, the biguanide, and the sulfonylurea in the pharmaceutical composition, wherein the dose of each the DPP IV inhibitor, the biguanide, and the sulfonylurea is about 70% of the lowest diabetic therapeutic dose. In some embodiments, treatment results in an improvement in diabetes and/or associated conditions that is greater than or equal to the improvement obtained with a combination of any two of the DPP IV inhibitor, the biguanide, and the sulfonylurea in the pharmaceutical composition, wherein the dose of the DPP IV inhibitor and the biguanide are about 70% of the lowest diabetic therapeutic dose for each of the DPP IV inhibitor and the biguanide, and the dose of the sulfonylurea is about 50% of the lowest diabetic therapeutic dose for the sulfonylurea.

In some embodiments, the treatment results in greater long term tolerability and reduced risk of side effects when compared to treatment with a combination of any two of the DPP IV inhibitor, the biguanide, and the sulfonylurea in the pharmaceutical composition, wherein the dose of each the DPP IV inhibitor, the biguanide, and the sulfonylurea is about 50% of the lowest diabetic therapeutic dose.

In some embodiments, the treatment is the initial or first-line treatment of diabetes. In some embodiments, the subject has a very mild elevation of blood sugar prior to treatment. In some embodiments, the subject is not on any previous diabetic therapy prior to treatment. In some embodiments, the subject has a very mild elevation of blood sugar prior to treatment and is not on any previous diabetic therapy prior to treatment. In some embodiments, the subject has persisting elevation of blood sugar after treatment with one or two of a DPP IV inhibitor, a biguanide, or a sulfonylurea at the LDTD or higher dose.

This present disclosure recognizes that the use of the DPP IV inhibitor in the pharmaceutical compositions disclosed herein in some embodiments provides beneficial therapeutic effects, which include, but are not limited to, significant reduction in blood sugar, significant reduction in blood sugar among subjects with mild elevation in blood sugar, greater long term tolerability, and reduced risk of side effects.

It is also recognized herein that in some embodiments, the triple low-dose combination formulation described herein comprising a DPP IV inhibitor, a biguanide, and a sulfonylurea provides reductions in blood sugar greater than the LDTD of each individual drug given singly. For example, in some embodiments, a triple combination formulation comprising 70% DPP IV inhibitor, 50% biguanide, and 70% sulfonylurea provides reductions in blood sugar greater than, or substantially greater than, the LDTD of the DPP IV inhibitor, or the LDTD of biguanide, or the LDTD of the sulfonylurea, given singly. As another example, in some embodiments, a triple combination formulation comprising 50% DPP IV inhibitor, 50% biguanide, and 50% sulfonylurea provides reductions in blood sugar greater than, or substantially greater than, the LDTD of the DPP IV inhibitor, or the LDTD of biguanide or the LDTD of the sulfonylurea, given singly.

It is also recognized herein that in some embodiments, the triple low-dose combination formulation described herein comprising a DPP IV inhibitor, a biguanide, and a sulfonylurea provides reductions in blood sugar greater than twice the LDTD of each individual drug given singly. For example, in some embodiments, a triple combination formulation comprising 70% DPP IV inhibitor, 50% biguanide, and 70% sulfonylurea provides reductions in blood sugar greater than, or substantially greater than twice the LDTD of each individual drug given singly. As another example, in some embodiments, a triple combination formulation comprising 50% DPP IV inhibitor, 50% biguanide, and 50% sulfonylurea provides reductions in blood sugar greater than, or substantially greater than twice the LDTD of each individual drug given singly. The disclosure will be further understood by the following non-limiting examples.

### NUMBERED EMBODIMENTS

Numbered embodiment 1 comprises a pharmaceutical composition, comprising: a) a low-dose, therapeutically-effective amount of a dipeptidyl peptidase IV (DPP IV) inhibitor; b) a low-dose, therapeutically-effective amount of a biguanide; c) a low-dose, therapeutically-effective amount of a sulfonylurea; and d) at least one pharmaceutically-acceptable excipient, wherein (a), (b), and (c) are each at about 20% to about 75% of a lowest diabetes therapeutic dose (LDTD). Numbered embodiment 2 comprises the pharmaceutical composition of numbered embodiment 1, wherein the DPP IV inhibitor is a gliptin. Numbered embodiment 3 comprises the pharmaceutical composition of numbered embodiments 1-2, wherein the DPP IV inhibitor is sitagliptin, vildagliptin, saxagliptin, linagliptin, gemigliptin, anagliptin, teneligliptin, alogliptin, trelagliptin, omarigliptin, evogliptin, gosogliptin, dutogliptin, or a pharmaceutically acceptable salt or hydrate thereof. Numbered embodiment 4 comprises the pharmaceutical composition of numbered embodiments 1-3, wherein the DPP IV inhibitor is sitagliptin or a pharmaceutically acceptable salt thereof. Numbered embodiment 5 comprises the pharmaceutical composition of numbered embodiments 1-4, wherein the DPP IV inhibitor is sitagliptin phosphate. Numbered embodiment 6 comprises the pharmaceutical composition of numbered embodiments 1-5, wherein the biguanide is metformin or a pharmaceutically acceptable salt or hydrate thereof. Numbered embodiment 7 comprises the pharmaceutical composition of numbered embodiments 1-6, wherein the biguanide is metformin hydrochloride. Numbered embodiment 8 comprises the pharmaceutical composition of numbered embodiments 1-7, wherein the metformin is formulated for immediate release. Numbered embodiment 9 comprises the pharmaceutical composition of numbered embodiments 1-8, wherein the metformin is formulated for slow release. Numbered embodiment 10 comprises the pharmaceutical composition of numbered embodiments 1-9, wherein sulfonylurea is acetohexamide, carbutamide, chlorpropamide, glycyclamide (tolhexamide), metahexamide, tolazamide, tolbutamide, glibenclamide (glyburide), glibornuride, gliclazide, glipizide, gliquidone, glisoxepide, glyclopyramide, glimepiride, or a pharmaceutically acceptable salt or hydrate thereof. Numbered embodiment 11 comprises the pharmaceutical composition of numbered embodiments 1-10, wherein the sulfonylurea is glimepiride. Numbered embodiment 12 comprises the pharmaceutical composition of numbered embodiments 1-11, wherein a dose of each (a), (b), and (c) is from about 40% to about 70% of the lowest diabetes therapeutic dose (LDTD). Numbered embodiment 13 comprises the pharmaceutical composition of numbered embodiments 1-12, wherein a dose of each (a), (b), and (c) is from about 40% to about 60% of the lowest diabetes therapeutic dose (LDTD). Numbered embodiment 14 comprises the pharmaceutical composition of numbered embodiments 1-13, wherein a dose of each (a), (b), and (c) is from about 45% to about 55% of the lowest diabetes therapeutic dose (LDTD). Numbered embodiment 15 comprises the pharmaceutical composition of numbered embodiments 1-14, wherein the DPP IV inhibitor is about 70% of the lowest diabetes therapeutic dose (LDTD) for the DPP IV inhibitor. Numbered embodiment 16 comprises the pharmaceutical composition of numbered embodiments 1-15, wherein the DPP IV inhibitor is sitagliptin, and a dose of sitagliptin is about 17.5 mg. Numbered embodiment 17 comprises the pharmaceutical composition of numbered embodiments 1-16, wherein the DPP IV inhibitor is about 50% of the lowest diabetes therapeutic dose (LDTD) for the DPP IV inhibitor. Numbered embodiment 18 comprises the pharmaceutical composition of numbered embodiments 1-17, wherein the DPP IV inhibitor is sitagliptin, and a dose of sitagliptin is about 12.5 mg. Numbered embodiment 19 comprises the pharmaceutical composition of numbered embodiments 1-18, wherein the biguanide is about 70% of the lowest diabetes therapeutic dose (LDTD) for the biguanide. Numbered embodiment 20 comprises the pharmaceutical composition of numbered embodiments 1-19, wherein the biguanide is metformin hydrochloride, and a dose of metformin hydrochloride is about 350 mg. Numbered embodiment 21 comprises the pharmaceutical composition of numbered embodiments 1-20, wherein the biguanide is about 50% of the lowest diabetes therapeutic dose (LDTD) for the biguanide. Numbered embodiment 22 comprises the pharmaceutical composition of numbered embodiments 1-21, wherein the biguanide is metformin hydrochloride, and a dose of metformin hydrochloride is about 250 mg. Numbered embodiment 23 comprises the pharmaceutical composition of numbered embodiments 1-22, wherein the sulfonylurea is about 50% of the lowest diabetes therapeutic dose (LDTD) for the sulfonylurea. Numbered embodiment 24 comprises the pharmaceutical composition of numbered embodiments 1-23, wherein the sulfonylurea is glimepiride, and a dose of the glimepiride is about 0.5 mg. Numbered embodiment 25 comprises the pharmaceutical composition of numbered embodiments 1-24, wherein the DPP IV inhibitor is sitagliptin, the biguanide is metformin, and the sulfonylurea is glimepiride. Numbered embodiment 26 comprises the pharmaceutical composition of numbered embodiments 1-25, wherein a dose of sitagliptin is from about 5.0 mg to about 18.75 mg, a dose of metformin is from about 100 mg to about 375 mg, and a dose of glimepiride is from about 0.2 mg to about 0.75 mg. Numbered embodiment 27 comprises the pharmaceutical composition of numbered embodiments 1-28, wherein the dose of sitagliptin is from about 10 mg to about 16.25 mg, the dose of metformin is from about 200 mg to about 325 mg, and the dose of glimepiride is from about 0.4 mg to about 0.65 mg. Numbered embodiment 28 comprises the pharmaceutical composition of numbered embodiments 1-27, wherein the dose of sitagliptin is from about 10 mg to about 15 mg, the dose of metformin is from about 200 mg to about 300 mg, and the dose of glimepiride is from about 0.4 mg to about 0.6 mg. Numbered embodiment 29 comprises the pharmaceutical composition of numbered embodiments 1-28, wherein the dose of sitagliptin is from about 11.25 mg to about 13.75 mg, the dose of metformin is from about 225 mg to about 275 mg, and the dose of glimepiride is from about 0.45 mg to about 0.55 mg. Numbered embodiment 30 comprises the pharmaceutical composition of numbered embodiments 1-29, wherein the dose of sitagliptin is about 12.5 mg, the dose of metformin is about 250 mg, and the dose of glimepiride is about 0.5 mg. Numbered embodiment 31 comprises the pharmaceutical composition of numbered embodiments 1-30, wherein the dose of each (a), (b), and (c) is from about 30% to about 40% of the lowest diabetes therapeutic dose (LDTD). Numbered embodiment 32 comprises the pharmaceutical composition of numbered embodiments 1-31, wherein the dose of each (a), (b), and (c) is from about 30% to about 35% of the lowest diabetes therapeutic dose (LDTD). Numbered embodiment 33 comprises the pharmaceutical composition of numbered embodiments 1-32, wherein the sulfonylurea is about 33% of the lowest diabetes therapeutic dose (LDTD) for the sulfonylurea. Numbered embodiment 34 comprises the pharmaceutical composition of numbered embodiments 1-33, wherein the sulfonylurea is glimepiride, and the dose of sulfonylurea is about 0.33 mg. Numbered embodiment 35 comprises the pharmaceutical composition of numbered embodiments 1-34, wherein the DPP IV inhibitor is sitagliptin, the biguanide is metformin, and the sulfonylurea is glimepiride. Numbered embodiment 36 comprises the pharmaceutical composition of numbered embodiments 1-35, wherein the dose of sitagliptin is from about 7.5 mg to about 10 mg, the dose of metformin is from about 150 mg to about 200 mg, and the dose of glimepiride is from about 0.3 mg to about 0.4 mg. Numbered embodiment 37 comprises the pharmaceutical composition of numbered embodiments 1-36, wherein the dose of sitagliptin is about 8.25 mg, the dose of metformin is about 165 mg, and the dose of glimepiride is about 0.33 mg. Numbered embodiment 38 comprises the pharmaceutical composition of numbered embodiments 1-37, wherein the dose of each (a), (b), and (c) is from about 20% to about 30% of the lowest diabetes therapeutic dose (LDTD). Numbered embodiment 39 comprises the pharmaceutical composition of numbered embodiments 1-38, wherein the dose of each (a), (b), and (c) is from about 22% to about 28% of the lowest diabetes therapeutic dose (LDTD). Numbered embodiment 40 comprises the pharmaceutical composition of numbered embodiments 1-39, wherein the sulfonylurea is about 25% of the lowest diabetes therapeutic dose (LDTD) for the sulfonylurea. Numbered embodiment 41 comprises the pharmaceutical composition of numbered embodiments 1-40, wherein the sulfonylurea is glimepiride, and the dose of sulfonylurea is about 0.25 mg. Numbered embodiment 42 comprises the pharmaceutical composition of numbered embodiments 1-41, wherein the DPP IV inhibitor is sitagliptin, the biguanide is metformin, and the sulfonylurea is glimepiride. Numbered embodiment 43 comprises the pharmaceutical composition of numbered embodiments 1-42, wherein the dose of sitagliptin is from about 5 mg to about 7.5 mg, the dose of metformin is from about 100 mg to about 150 mg, and the dose of glimepiride is from about 0.2 mg to about 0.3 mg. Numbered embodiment 44 comprises the pharmaceutical composition of numbered embodiments 1-43, wherein the dose of sitagliptin is about 6.25 mg, the dose of metformin is about 150 mg, and the dose of glimepiride is about 0.25 mg. Numbered embodiment 45 comprises the pharmaceutical composition of numbered embodiments 1-44, wherein the pharmaceutical composition is in the form of pill, tablet, or capsule. Numbered embodiment 46 comprises the pharmaceutical composition of numbered embodiments 1-45, wherein the pharmaceutical composition is suitable for oral administration. Numbered embodiment 47 comprises the pharmaceutical composition of numbered embodiments 1-46, wherein the pharmaceutical composition does not comprise any further additional anti-hyperglycemic or anti-diabetic agents. Numbered embodiment 48 comprises the pharmaceutical composition of numbered embodiments 1-47, wherein the combination of a), b), and c) produces a synergistic effect. Numbered embodiment 49 comprises the pharmaceutical composition of numbered embodiments 1-48, wherein the pharmaceutical composition produces a larger decrease in 2 hour post-prandial glucose as compared to a maximum decrease in the post-prandial glucose obtained from about 100 mg of sitagliptin. Numbered embodiment 50 comprises the pharmaceutical composition of numbered embodiments 1-49, wherein the pharmaceutical composition produces a larger decrease in 2 hour post-prandial glucose as compared to a maximum decrease in the post-prandial glucose obtained from about 850 mg of metformin. Numbered embodiment 51 comprises the pharmaceutical composition of numbered embodiments 1-50, wherein the pharmaceutical composition produces a larger decrease in 2 hour post-prandial glucose as compared to a maximum decrease in the post-prandial glucose obtained from about 1700 mg of metformin. Numbered embodiment 52 comprises a pharmaceutical composition, comprising: a) a low-dose, therapeutically-effective amount of a dipeptidyl peptidase IV (DPP IV) inhibitor; b) a low-dose, therapeutically-effective amount of a biguanide; c) a low-dose, therapeutically-effective amount of a sulfonylurea; and d) at least one pharmaceutically-acceptable excipient, wherein (a) and (b) are each at about 65%-75% of a lowest diabetes therapeutic dose (LDTD), and (c) is at about 45%-55% of the lowest diabetes therapeutic dose (LDTD). Numbered embodiment 53 comprises the pharmaceutical composition of numbered embodiments 1-52, wherein the DPP IV inhibitor is sitagliptin and a dose of sitagliptin is from about 16.25 mg to about 18.75 mg. Numbered embodiment 54 comprises the pharmaceutical composition of numbered embodiments 1-53, wherein the biguanide is metformin and a dose of metformin is from about 325 mg to about 375 mg. Numbered embodiment 55 comprises the pharmaceutical composition of numbered embodiments 1-54, wherein the sulfonylurea is glimepiride, and a dose of glimepiride from about 0.45 mg to about 0.55 mg. Numbered embodiment 56 comprises the pharmaceutical composition of numbered embodiments 1-55, wherein the DPP IV inhibitor is at about 70% of the lowest diabetes therapeutic dose (LDTD) for the DPP IV inhibitor. Numbered embodiment 57 comprises the pharmaceutical composition of numbered embodiments 1-56, wherein the biguanide is at about 70% of the lowest diabetes therapeutic dose (LDTD) for the biguanide. Numbered embodiment 58 comprises the pharmaceutical composition of numbered embodiments 1-57, wherein the sulfonylurea is at about 50% of the lowest diabetes therapeutic dose (LDTD) for the sulfonylurea. Numbered embodiment 59 comprises the pharmaceutical composition of numbered embodiments 1-58, wherein the DPP IV inhibitor is sitagliptin, the biguanide is metformin, and the sulfonylurea is glimepiride. Numbered embodiment 60 comprises the pharmaceutical composition of numbered embodiments 1-59, wherein the dose of sitagliptin is about 17.5 mg, the dose of metformin is about 350 mg, and the dose of glimepiride is about 0.5 mg. Numbered embodiment 61 comprises the pharmaceutical composition of numbered embodiments 1-60, wherein the DPP IV inhibitor is sitagliptin and the dose of the sitagliptin is about 17.5 mg. Numbered embodiment 62 comprises the pharmaceutical composition of numbered embodiments 1-61, wherein biguanide is metformin and the dose of the metformin is about 350 mg. Numbered embodiment 63 comprises the pharmaceutical composition of numbered embodiments 1-62, wherein the sulfonylurea is glimepiride and the dose of the glimepiride is about 0.5 mg. Numbered embodiment 64 comprises the pharmaceutical composition of numbered embodiments 1-63, wherein the pharmaceutical composition is suitable for oral administration. Numbered embodiment 65 comprises the pharmaceutical composition of numbered embodiments 1-64, wherein the pharmaceutical composition is in the form of pill, tablet or capsule. Numbered embodiment 66 comprises the pharmaceutical composition of numbered embodiments 1-65, wherein the metformin is formulated for immediate release. Numbered embodiment 67 comprises the pharmaceutical composition of numbered embodiments 1-66, wherein the metformin is formulated for slow release. Numbered embodiment 68 comprises the pharmaceutical composition of numbered embodiments 1-67, wherein the pharmaceutical composition does not comprise any further additional anti-hyperglycemic or anti-diabetic agents. Numbered embodiment 69 comprises the pharmaceutical composition of numbered embodiments 1-68, wherein the combination of a), b), and c) produces a synergistic effect. Numbered embodiment 70 comprises the pharmaceutical composition of numbered embodiments 1-69, wherein the pharmaceutical composition produces a larger decrease in 2 hour post-prandial glucose as compared to a maximum decrease in the post-prandial glucose obtained from about 100 mg of sitagliptin. Numbered embodiment 71 comprises the pharmaceutical composition of numbered embodiments 1-70, wherein the pharmaceutical composition produces a larger decrease in 2 hour post-prandial glucose as compared to a maximum decrease in the post-prandial glucose obtained from about 850 mg of metformin. Numbered embodiment 72 comprises the pharmaceutical composition of numbered embodiments 1-71, wherein the pharmaceutical composition produces a larger decrease in 2 hour post-prandial glucose as compared to a maximum decrease in the post-prandial glucose obtained from about 1700 mg of metformin. Numbered embodiment 73 comprises a pharmaceutical composition, comprising a combination of: a) about 17.5 mg of sitagliptin; b) about 350 mg of metformin; c) about 0.5 mg of glimepiride; and d) at least one pharmaceutically-acceptable excipient. Numbered embodiment 74 comprises the pharmaceutical composition of numbered embodiments 1-73, wherein the combination is synergistic. Numbered embodiment 75 comprises the pharmaceutical composition of numbered embodiments 1-74, wherein the pharmaceutical composition is in the form of pill, tablet, or capsule. Numbered embodiment 76 comprises the pharmaceutical composition of numbered embodiments 1-75, wherein the pharmaceutical composition is suitable for oral administration. Numbered embodiment 77 comprises the pharmaceutical composition of numbered embodiments 1-76, wherein the metformin is formulated for immediate release Numbered embodiment 78 comprises the pharmaceutical composition of numbered embodiments 1-77, wherein the metformin is formulated for slow release. Numbered embodiment 79 comprises the pharmaceutical composition of numbered embodiments 1-78, wherein the pharmaceutical composition does not comprise any further additional anti-hyperglycemic or anti-diabetic agents. Numbered embodiment 80 comprises the pharmaceutical composition of numbered embodiments 1-79, wherein the combination of a), b), and c) produces a synergistic effect. Numbered embodiment 81 comprises the pharmaceutical composition of numbered embodiments 1-80, wherein the pharmaceutical composition produces a larger decrease in 2 hour post-prandial glucose as compared to a maximum decrease in the post-prandial glucose obtained from about 100 mg of sitagliptin. Numbered embodiment 82 comprises the pharmaceutical composition of numbered embodiments 1-81, wherein the pharmaceutical composition produces a larger decrease in 2 hour post-prandial glucose as compared to a maximum decrease in the post-prandial glucose obtained from about 850 mg of metformin. Numbered embodiment 83 comprises the pharmaceutical composition of numbered embodiments 1-82, wherein the pharmaceutical composition produces a larger decrease in 2 hour post-prandial glucose as compared to a maximum decrease in the post-prandial glucose obtained from about 1700 mg of metformin. Numbered embodiment 84 comprises a synergistic, ultra-low dose, anti-diabetic drug combination, consisting of: a) about 16.25 mg to about 18.75 mg of sitagliptin, or a salt or hydrate thereof; b) about 325 mg to about 375 mg of metformin, or a salt or hydrate thereof; c) about 0.45 mg to about 0.55 mg of glimepiride, or a salt or hydrate thereof; and d) at least one excipient. Numbered embodiment 85 comprises the combination of numbered embodiments 1-84, wherein the combination does not comprise any further additional anti-hyperglycemic or anti-diabetic agents. Numbered embodiment 86 comprises the combination of numbered embodiments 1-85, wherein the combination of a), b), and c) produces a synergistic effect. Numbered embodiment 87 comprises the combination of numbered embodiments 1-86, wherein the combination produces a larger decrease in 2 hour post-prandial glucose as compared to a maximum decrease in the post-prandial glucose obtained from about 100 mg of sitagliptin, from about 850 mg of metformin, or from about 1700 mg of metformin. Numbered embodiment 88 comprises a method of treating diabetes in a subject in need thereof comprising administering the pharmaceutical composition as described herein. Numbered embodiment 89 comprises the method of numbered embodiments 1-88, wherein the subject has persisting elevation of blood sugar after treatment with one or two of a DPP IV inhibitor, a biguanide, or a sulfonylurea at the LDTD or higher dose. Numbered embodiment 90 comprises the method of numbered embodiments 1-89, wherein the administration of the pharmaceutical composition is an initial or first-line treatment of diabetes. Numbered embodiment 91 comprises a method of improving, slowing the progression of, or delaying a metabolic disorder, wherein the metabolic disorder comprises diabetes mellitus, impaired glucose tolerance, impaired fasting blood glucose, hyperglycemia, postprandial hyperglycemia, overweight, obesity, metabolic syndrome, impaired renal function, gestational diabetes, new onset diabetes after transplantation (NODAT) and complications associated therewith, or post-transplant metabolic syndrome (PTMS) and complications associated therewith, comprising administering to a subject in need thereof the pharmaceutical composition as described herein. Numbered embodiment 92 comprises a method of treating diabetes in a subject in need thereof comprising administering a synergistic, ultra-low dose, anti-diabetic drug combination, consisting of: a) about 16.25 mg to about 18.75 mg of sitagliptin, or a salt or hydrate thereof; b) about 325 mg to about 375 mg of metformin, or a salt or hydrate thereof; c) about 0.45 mg to about 0.55 mg of glimepiride, or a salt or hydrate thereof; and d) at least one excipient. Numbered embodiment 93 comprises the method of numbered embodiments 1-92, wherein the combination does not comprise any further additional anti-hyperglycemic or anti-diabetic agents. Numbered embodiment 94 comprises the method of numbered embodiments 1-93, wherein the combination of a), b), and c) produces a synergistic effect. Numbered embodiment 95 comprises the method of numbered embodiments 1-94, wherein the combination produces a larger decrease in 2 hour post-prandial glucose as compared to a maximum decrease in the post-prandial glucose obtained from about 100 mg of sitagliptin, from about 850 mg of metformin, or from about 1700 mg of metformin.

### EXAMPLES

The examples set forth below are provided to give those of ordinary skill in the art with a complete disclosure and description of how to make and use the claimed embodiments, and are not intended to limit the scope of what is disclosed herein. Modifications that are obvious to persons of skill in the art are intended to be within the scope of the following claims.

### Example 1

### Study of a Combination of a DPP IV Inhibitor, Biguanide, and Sulfonylurea in Patients with Diabetes Mellitus

The purpose of this study is to evaluate the safety and effectiveness sitagliptin, metformin or metformin hydrochloride, and glimepiride in patients with diabetes mellitus.

Patient entry criteria

Inclusion criteria: Type 2 diabetes mellitus; On no drug treatment or on a single oral agent; If on no drug, fasting blood glucose (FBG)≥8 mmol/l and glycosylated hemoglobin (HbAlc)≥7.0 mmol/l; If on one drug, FBG≥7.5 mmol/l and HbAlc≥6.5 mmol/l.

Exclusion criteria: FBG>10 mmol or HbAlc>8.5 mmol; Glomerular filtration rate (GFR)<45 mL/min; Clinical history of microvascular disease or neuropathy; Any contraindication to treatment with a DPP-IV inhibitor, biguanide or sulfonylurea.

Study treatments:

Study treatment A: A fixed dose combination of sitagliptin 5-7.5 mg (20%-30% of LDTD), glimepiride 0.2-0.3 mg (20%-30% of LDTD), and metformin 100-150 mg (20%-30% of LDTD); or matching placebo and taken once a day in the morning.

Study treatment B: A fixed dose combination of sitagliptin 7.5-10 mg (30%-40% of LDTD), glimepiride 0.3-0.4 mg (30%-40% of LDTD), and metformin 150-200 mg (30%-40% of LDTD); or matching placebo and taken once a day in the morning.

Study treatment C: A fixed dose combination of sitagliptin 11.25-12.5 mg (45%-55% of LDTD), glimepiride 0.45-0.55 mg (45%-55% of LDTD), and metformin 225-275 mg (45%-55% of LDTD); or matching placebo and taken once a day in the morning.

Study treatment D: A fixed dose combination of sitagliptin 15-18.75 mg (60%-75% of LDTD), glimepiride 0.60-0.75 mg (60%-75% of LDTD), and metformin 300-375 mg (60%-75% of LDTD); or matching placebo and taken once a day in the morning.

Study treatment E:

TABLE 5
Treatment groups and doses in ultra-low-
dose glucose-lowering drug combinations

| | | | Human | Animal | Animal | |
|---|---|---|---|---|---|---|
| | | | Dose | Dose | Concen- | Animal |
| | | | Level | Level | tration | Volume |
| Group | Test Article | | (mg) | (mg/kg) | (mg/mL) | (mL/kg) |
| 1 | Vehicle | Metformin | 0 | 0 | 0 | 10 |
| 2 | Compo- | Glimep- | 0.5 | 0.044 | 0.0044 | 10 |
| | sition A | iride | | | | |
| | | Sita- | 17.5 | 1.545 | 0.1545 | |
| | | gliptin | | | | |
| | | Metformin | 350 | 30.833 | 3.0833 | |
| 3 | 1K-MET | Metformin | 1000 | 88.095 | 8.8095 | 10 |

Taken once a day in the morning

Treatment duration: 6 weeks

Study Outcomes

Primary: FBG

Secondary: Continuous blood glucose (CBG) values; Tolerability and safety (T&S); Particularly, hypoglycemic episodes diagnosed on the basis of symptoms or continuous blood glucose values; Adherence; Percent tablets taken (ADH).

Statistical Considerations

Assumptions regarding primary outcome for each study treatment-placebo comparison: Initial FBG level: mean=8 mmol/l; SD=2; Difference between study treatment and placebo groups at end of follow-up: 1.2 mmol/l; Beta=0.05; Alpha=0.85. Required sample size=100.

### Example 2

### Study of a Combination of a DPP IV Inhibitor, Biguanide, and Sulfonylurea in Patients with Diabetes Mellitus

The purpose of this study is to evaluate the safety and effectiveness sitagliptin, metformin or metformin hydrochloride, and glimepiride in patients with diabetes mellitus.

Patient Entry Criteria

Inclusion criteria: Type 2 diabetes mellitus; On no drug treatment or on a single oral agent; If on no drug, fasting blood glucose (FBG)≥8 mmol/l and glycosylated hemoglobin (HbAlc)≥7.0 mmol/l; If on one drug, FBG≥7.5 mmol/l and HbAlc≥6.5 mmol/l.

Exclusion criteria: FBG>10 mmol or HbAlc>8.5 mmol; Glomerular filtration rate (GFR)<45 mL/min; Clinical history of microvascular disease or neuropathy; Any contraindication to treatment with a DPP-4 inhibitor, biguanide or sulfonylurea.

Study treatment: A fixed dose combination of sitagliptin 17.5 mg (70% of LDTD), glimepiride 0.5 mg (50% of LDTD), and metformin 350 mg (70% of LDTD); or matching placebo and taken once a day in the morning.

Treatment duration: 6 weeks

Study Outcomes

Primary: FBG

Secondary: Continuous blood glucose (CBG) values; Tolerability and safety (T&S); Particularly, hypoglycemic episodes diagnosed on the basis of symptoms or continuous blood glucose values; Adherence; Percent tablets taken (ADH).

Statistical Considerations

Assumptions regarding primary study outcome: Initial FBG level: mean=8 mmol/L; SD=2; Difference between study treatment and placebo groups at end of follow-up: 1.2 mmol/L; Beta=0.05; Alpha=0.85. Required sample size=100.

### Example 3

### Analysis of Glucose and Insulin Profiles in Patients with Type 2 Diabetes

The purpose of this study was to evaluate the pharmacodynamics (glucose-lowering effects) and tolerability of Composition A in patients with diabetes mellitus.

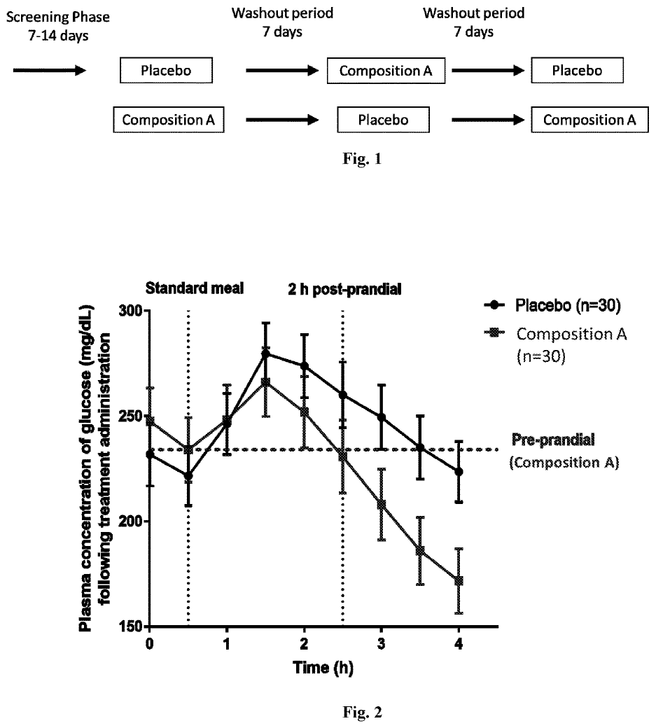

In the present study, thirty adult patients with type 2 diabetes (23 females and 7 males), either treatment naive, previously treated or currently treated with only one glucose lowering therapeutic class excluding insulin and glucagon like peptide-1 analogues, were randomized to receive either placebo or Composition A comprising 350 mg of metformin, 17.5 mg of sitagliptin, and 0.5 mg of glimepiride in a 3 treatment, 3 sequence, 3 period, crossover study, with one week washout in between periods as shown in FIG. 1. The primary and secondary endpoints were the mean absolute change in plasma glucose and serum insulin respectively at 2 hour (h) post-prandial from pre-prandial following the administration of a single dose of the treatments. The tertiary outcomes were the 120-minutes and 210-minutes of the area under the concentration-time curve (AUC) of plasma glucose and serum insulin post-meal, and the 120-minutes and 240-minutes of the area under the concentration-time curve (AUC) of plasma glucose and serum insulin post-dose. An ad hoc analysis was performed to determine the differences between the study groups in the mean absolute change in plasma glucose and serum insulin from pre-prandial at the following time points after the administration of a single dose of the study treatments: 1, 1.5, 2, 2.5, 3, 3.5 and 4 h.

Participating patients withheld taking any glucose-lowering therapy 24 h prior to the screening visit and for 7 days prior to the study visits and until the study exit. During the study visits, blood samples were collected from the participants before the administration of a single dose of the randomized treatment and at the following times after treatment administration: 0.5, 1, 1.5, 2, 2.5, 3, 3.5 and 4 h. Participants consumed a standard meal (550 kcal; composed of 55% carbohydrate, 15% protein, and 30% fat) immediately after the 0.5 h blood collection. Measurement of plasma glucose and serum insulin were performed on all samples by validated methods. Plasma glucose was measured.

Methods

Statistical Analysis

Data for each treatment group were pooled. A one-way analysis of variance (ANOVA) was performed to examine the differences between treatments in the primary (mean absolute change in plasma glucose at 2 h post-prandial from pre-prandial) and secondary endpoint (mean absolute change in serum insulin at 2 h post-prandial from pre-prandial) using SPSS version 17.0. Post hoc multiple pairwise comparisons were performed using the Tukey test. The AUC₁₂₀ₘᵢₙ and AUC₂₁₀ₘᵢₙ of glucose and insulin post-meal and the AUC₁₂₀ₘᵢₙ and AUC₂₄₀ₘᵢₙ post-dose were calculated using the standard trapezoidal method. WinNonlin version 8.2 was used to compare pairs of the study treatments and to estimate the sequence and period effects. An ad hoc analysis using R Statistical Programming version 3.6.1 was performed to determine the differences between the study groups in the mean absolute change in plasma glucose and serum insulin from pre-prandial concentrations (0.5 h collection time point) at the following time points after the administration of a single dose of the study treatments: 1, 1.5, 2, 2.5, 3, 3.5 and 4 h. A p-value<0.05 was considered statistically significant.

Results

The baseline characteristics are summarized in Table 6. Thirty patients with type 2 diabetes were recruited in the present study. Baseline plasma glycated hemoglobin (HBA1c) and fasting plasma glucose in the participating patients ranged from ≥6.1% to ≤11.9% and >119 to 260 mg/dL, respectively (Table 6).

TABLE 6
Demographics and characteristics of the study population at screening

| | | | | | | Duration | | | |
|---|---|---|---|---|---|---|---|---|---|
| | | | Body | | | of type 2 | | | eGFR |
| Subject | Gender | Age | weight | Height | BMI | diabetes | HBA_{1c} | FPG | (mL/min/ |
| ID | (M/F) | (y) | (kg) | (cm) | (kg/m²) | (y) | (%) | (mg/dL) | 1.73 m²) |
| S-01 | F | 59 | 64 | 148 | 29.2 | 3 | 10.7 | 180 | 137 |
| S-02 | M | 47 | 76 | 161 | 29.3 | 6 | 7.5 | 199 | 145 |
| S-03 | F | 44 | 75 | 147 | 34.7 | 4 | 11.9 | 206 | 196 |
| S-04 | F | 48 | 115 | 148 | 52.5 | 19 | 8.4 | 169 | 125 |
| S-05 | F | 53 | 59 | 148 | 26.9 | 2 | 11.6 | 198 | 151 |
| S-06 | F | 52 | 45 | 142 | 22.3 | 1 | 8.1 | 129 | 72 |
| S-07 | M | 56 | 51 | 158 | 20.4 | 3 | 9.3 | 188 | 157 |
| S-08 | M | 62 | 76 | 161 | 29.3 | 2 | 7 | 139 | 92 |
| S-09 | F | 45 | 50 | 135 | 27.4 | 2 | 8.4 | 190 | 142 |
| S-10 | M | 43 | 74 | 170 | 25.6 | 3 | 6.5 | 157 | 73 |
| S-11 | F | 35 | 66 | 160 | 25.8 | 1 | 8.5 | 221 | 156 |
| S-12 | F | 59 | 52 | 154 | 21.9 | 1 | 7.1 | 159 | 107 |
| S-13 | F | 46 | 54 | 156 | 22.2 | 2 | 10.3 | 219 | 148 |
| S-14 | F | 48 | 60 | 149.5 | 26.9 | 2 | 11 | 190 | 48 |
| S-15 | F | 46 | 64 | 152 | 27.7 | 10 | 7.9 | 149 | 132 |
| S-16 | F | 53 | 60 | 157 | 24.3 | 3 | 10.9 | 235 | 183 |
| S-17 | F | 50 | 71 | 156 | 29.2 | 9 | 10.5 | 240 | 165 |
| S-18 | F | 54 | 51 | 141 | 25.7 | 6 | 7.9 | 157 | 120 |
| S-19 | F | 55 | 54 | 152 | 23.4 | 1 | 11.2 | 223 | 97 |
| S-20 | M | 46 | 105 | 167 | 37.7 | 5 | 6.1 | 120 | 108 |
| S-21 | F | 43 | 58 | 150.5 | 25.6 | 3 | 9.1 | 160 | 180 |
| S-22 | M | 45 | 65 | 170 | 22.5 | 2 | 8.2 | 240 | 60 |
| S-23 | F | 43 | 104 | 161 | 40.1 | 3 | 10.3 | 234 | 112 |
| S-24 | F | 42 | 82 | 161 | 31.6 | 6 | 9.1 | 180 | 94 |
| S-25 | F | 41 | 103 | 155 | 42.9 | 1 | 6.6 | 119 | 129 |
| S-26 | F | 45 | 77 | 152 | 33.3 | 1 | 8.3 | 141 | 59 |
| S-27 | F | 51 | 55 | 149 | 24.8 | 1 | 10.1 | 260 | 132 |
| S-28 | F | 39 | 70 | 156 | 28.8 | 6 | 11.5 | 219 | 106 |
| S-29 | M | 54 | 66 | 165.5 | 24.1 | 3 | 8.2 | 199 | 79 |
| S-30 | F | 58 | 87 | 150.5 | 38.4 | 10 | 8.5 | 216 | 83 |
| Summary | 23 F (77%) | 48.7 ± 6.6 | 69.6 ± 18.1 | 154.4 ± 8.3 | 29.2 ± 7.2 | 4.0 ± 3.9 | 9.0 ± 1.7 | 188.1 ± 39.5 | 119.0 ± 39.6 |
| | 7 M (23%) | | | | | | | | |

a. Summary presented as number (percentage) or mean ± SD.
BMI = body mass index, eGFR = estimated glomerular filtration rate, FPG = fasting plasma glucose, HBA_{1c} = glycated hemoglobin.

In terms of efficacy, there was an overall statistically significant difference among treatments for the primary endpoint (p<0.05, Table 7), and the secondary endpoint (p<0.05, Table 8), and the tertiary outcomes (p<0.05, Table 9).

TABLE 7
The effect of a single dose of the study treatments on
the 2 hour post-prandial plasma glucose (2 h PPG) from
pre-prandial in patients with type 2 diabetes (n = 30).

| Comparison | Treatment | Estimate (95% CI) | | p-value |
|---|---|---|---|---|
| Pre-prandial | Placebo | 221.6 | (192.8, 250.4) | |
| plasma glucose | Composition A | 234.0 | (202.7, 265.3) | |
| (95% CI) | | | | |
| 2 h PPG | Placebo | 260.13 | (228.4, 291.9) | |
| (95% CI) | Composition A | 230.7 | (195.3, 266.0) | |
| Pairwise | Composition A | −41.9 | (−58.1, −25.6) | 7.4 × 10⁻⁸* |
| comparison | vs Placebo | | | |
| (Tukey Test) | | | | |

*Statistically significant.

TABLE 8
The effect of a single dose of the study treatments
on the 2 hour post-prandial serum insulin from pre-
prandial in patients with type 2 diabetes (n = 30).

| Comparison | Treatment | Estimate (95% CI) | | P-value |
|---|---|---|---|---|
| Pre-prandial serum | Placebo | 12.3 | (9.8, 14.7) | |
| insulin (95% CI) | Composition A | 14.8 | (10.4, 19.2) | |
| 2 h post-prandial | Placebo | 28.7 | (22.9, 34.6) | |
| serum insulin (95% CI) | Composition A | 47.7 | (36.8, 58.5) | |
| Pairwise comparison | Composition A | 16.4 | (−26.8, −6.0) | 0.0009* |
| (Tukey Test) | vs Placebo | | | |

*Statistically significant

TABLE 9
The tertiary endpoints following the administration of a single dose
of the study treatments in patients with type 2 diabetes (n = 30).

| | | | |
|---|---|---|---|
| Comparison | Treatment | Mean ± SD | P-value |

Area under the concentration-time
curve of plasma glucose (mg · h/dL)

| | | | |
|---|---|---|---|
| AUC₀₋₁₂₀ post-meal | Placebo | 520.3 ± 159.7 | |
| | Composition A | 499.2 ± 178.9 | |
| Pairwise comparison | Composition A | | 0.045* |
| | vs. Placebo | | |
| AUC0-210 post-meal | Placebo | 883.5 ± 281.3 | |
| | Composition A | 796.7 ± 310.3 | |
| Pairwise comparison | Composition A | | 0.0005* |
| | vs. Placebo | | |
| AUC₀₋₁₂₀ post-dose | Placebo | 500.2 ± 157.8 | |
| | Composition A | 499.0 ± 174.5 | |
| Pairwise comparison | Composition A | | 0.7 |
| | vs. Placebo | | |
| AUC₀₋₂₄₀ post-dose | Placebo | 996.8 ± 320.4 | |
| | Composition A | 917.1 ± 351.7 | |
| Pairwise comparison | Composition A | | 0.003* |
| | vs. Placebo | | |

Area under the concentration-time
curve (AUC) of serum insulin (μIU · h/L)

| | | | |
|---|---|---|---|
| AUC₀₋₁₂₀ post-meal | Placebo | 51.6 ± 24.8 | |
| | Composition A | 71.3 ± 46.7 | |
| Pairwise comparison | Composition A | | 0.0001* |
| | vs. Placebo | | |
| AUC₀₋₂₁₀ post-meal | Placebo | 87.4 ± 41.0 | |
| | Composition A | 132.1 ± 75.6 | |
| Pairwise comparison | Composition A | | 0.000001* |
| | vs. Placebo | | |
| AUC₀₋₁₂₀ post-dose | Placebo | 43.3 ± 20.8 | |
| | Composition A | 55.7 ± 38.3 | |
| Pairwise comparison | Composition A | | 0.004* |
| | vs. Placebo | | |
| AUC₀₋₂₄₀ post-dose | Placebo | 93.9 ± 44.0 | |
| | Composition A | 139.5 ± 80.4 | |
| Pairwise comparison | Composition A | | 0.000002* |
| | vs. Placebo | | |

*Statistically significant p-value < 0.05

In terms of safety and tolerability, no adverse events were associated with the administration of a single dose of either placebo or Composition A in patients with type 2 diabetes (n=30).

Effect on Plasma Glucose

Time course of plasma glucose: The time course of plasma glucose following a single dose of Composition A and placebo is presented in FIG. 2. In the placebo group, plasma glucose increased after the standard meal, as would be expected, by a mean of 58 mg/dL over the pre-prandial concentration, achieving its peak at the 1.5 h time point (1 h post-prandial), then plasma glucose started to decrease gradually returning to approximately the pre-prandial concentration at the 4 h post-dose time point. In the Composition A group, plasma glucose increased by a mean of 32.1 mg/dL (achieved at the 1.5 h collection time point) over the pre-prandial concentration, then plasma glucose decreased steadily until the last collection time point (4 h). By contrast to placebo, in the Composition A group, plasma glucose remained below the pre-prandial over the 2.5 to 4 h collection time points.

Primary endpoint: The primary endpoint was the mean absolute change in the 2 h post-prandial plasma glucose (2 h PPG) from pre-prandial. Composition A significantly reduced the plasma concentrations of glucose at this endpoint compared to placebo (Table 7 and FIG. 2). The difference in the mean absolute change in the 2 h PPG from pre-prandial between the Composition A and placebo groups was −41.9 mg/dL (p=7.4×10⁻⁸, Table 7).

Ad Hoc Analysis: Composition A achieved significantly lower plasma glucose over the entire 1.5-4 h sampling window compared to Placebo (Table 10). The differences in the mean absolute change in plasma glucose from pre-prandial at the 1.5 to 4 h post-dose time points were statistically significant between the Composition A and Placebo groups (Table 10). Over this time interval (1.5-4 h post-dose), the differences in the mean absolute change in plasma glucose from pre-prandial between the Composition A and placebo groups ranged from −25.9 to −64.3 mg/dL (Table 10). The glucose-lowering effects of Composition A peaked at the 4 h time point (Table 10).

TABLE 10
The effect of a single dose of the study treatments on
the absolute change in plasma glucose from pre-prandial
in patients with type 2 diabetes (n = 30).
Plasma glucose (mg/dL)

| | | |
|---|---|---|
| Collection | | |
| time points | Overall | Composition A |
| post-dose | p-value | vs Placebo |

| | | | | |
|---|---|---|---|---|
| 1 | h | 1.0 × 10⁻⁵* | −10.5 | (−21.7, 0.7; 0.07) |
| 1.5 | h | 2.1 × 10⁻¹²* | −25.9 | (−39.2, −12.7; 0.00003*) |
| 2 | h | 1.1 × 10⁻¹²* | −34.5 | (−49.9, −19.0; 0.000002*) |
| 2.5 | h | 1.2 × 10⁻¹³* | −41.9 | (−58.1, −25.6; 0.0000001*) |
| 3 | h | 1.1 × 10⁻¹³* | −53.9 | (−71.7, −36.0; 0.0000000*) |
| 3.5 | h | 9.1 × 10⁻¹⁴* | −61.5 | (−80.1 −42.8; 0.0000000*) |
| 4 | h | 1.4 × 10⁻¹²* | −64.3 | (−84.2 −44.4; 0.0000000*) |

*Statistically significant

Effect on Serum Insulin

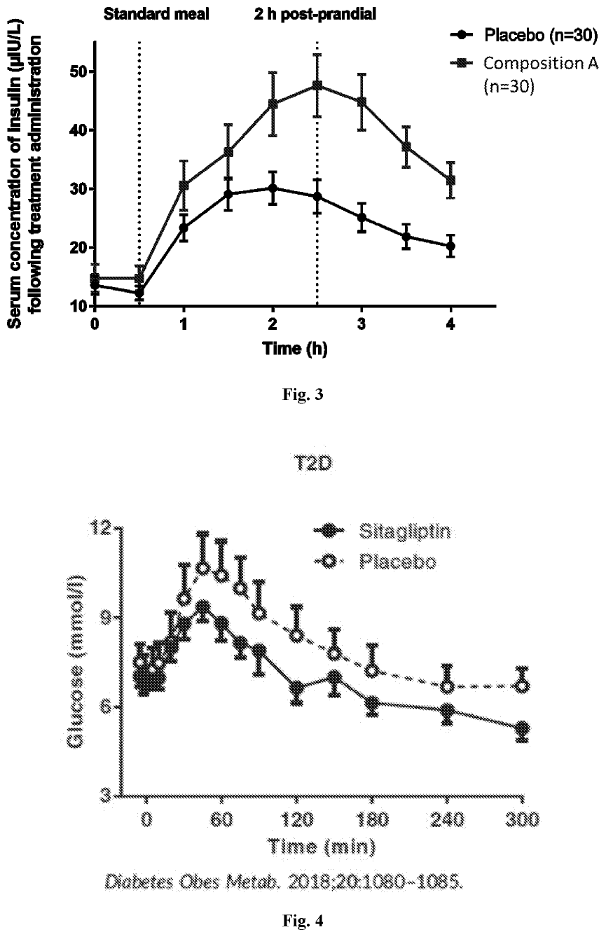

Time course of serum insulin: The Composition A group achieved higher concentrations of serum insulin compared to the placebo group as seen in FIG. 3. Serum insulin achieved its peak at the 2.5 h collection time point, then started to decrease gradually but did not recover to the pre-prandial concentration by the 4 h time point (FIG. 3). Likewise, in the placebo group, serum insulin did not recover to the pre-prandial concentration by the 4 h time point (FIG. 3). Secondary endpoint: The secondary endpoint was the mean absolute change in the 2 h post-prandial serum insulin from pre-prandial. Composition A significantly increased serum insulin from pre-prandial by 16.4 μIU/L at this time point compared to placebo (p=0.0009, Table 11 and FIG. 3).

TABLE 11
Serum insulin (μIU/mL)

| | | |
|---|---|---|
| Collection | | |
| time points | Overall | Composition A |
| post-dose | p-value | vs Placebo |

| | | | | |
|---|---|---|---|---|
| 1 | h | 0.2 | 4.7 | (−2.5; 12.0; 0.3) |
| 1.5 | h | 0.03* | 4.7 | (−3.8, 13.1; 0.4) |
| 2 | h | 0.0004* | 11.8 | (1.8, 21.8; 0.02*) |
| 2.5 | h | 1.7 × 10⁻⁵* | 16.4 | (6.04, 26.8; 0.0009*) |
| 3 | h | 2.0 × 10⁻⁷* | 17.2 | (8.2, 26.1; 0.00005*) |
| 3.5 | h | 1.5 × 10⁻⁶* | 12.8 | (5.8, 19.8; 0.0001*) |
| 4 | h | 3.1 × 10⁻⁶* | 8.7 | (2.7, 14.6; 0.002*) |

*Statistically significant

Ad hoc analysis: The difference in the mean absolute change in serum insulin from pre-prandial between the Composition A and placebo groups at the 2 to 4 h post-dose time points was statistically significant (Table 11). A maximum difference of 17.2 μIU/L was achieved between Composition A and placebo at the 3 h post-dose (Table 11).

Tertiary outcomes: Composition A achieved significantly lower area under the concentration-time curve (AUC) of plasma glucose over the entire blood sampling window compared to placebo. See Table 9. Composition A achieved a significantly higher area under the concentration-time curve (AUC) of serum insulin over the entire blood sampling window compared to placebo. See Table 9.

The data shows that Composition A has significant glucose-lowering effects and were not associated with any adverse events following a single dose administration in male and female patients with type 2 diabetes.

### Example 4

### Comparator Analysis

Time course of plasma glucose following single dose of monotherapy based on published studies was compared to Composition A.

Table 12 shows percentage change in plasma glucose from pre-prandial adjusted for placebo at various doses of single monotherapies as compared to Composition A.

TABLE 12
Percentage Change

| | At 2 h PPG | Maximum |
|---|---|---|
| Sitagliptin 100 mg | −6.3 | −20.3^{a} |
| Acarbose 100 mg | −5.3 | −24.5^{b} |
| Metformin 850 mg | −9.4 | Same as 2 h |
| Metformin 1700 mg | −21.9 | Same as 2 h |
| Composition A | −26.4 | |

^{a}Maximum achieved at 1.5 h post-prandial
^{b}Maximum achieved at 1 h post-prandial

As seen in FIG. 4, difference in mean 2 h post-prandial glucose (PPG) vs placebo (180 min) is ˜1 mmol/L. Baseline (fasting PG) was much lower compared to Composition A (drug-naïve in sitagliptin 100 mg). Maximum difference between sitagliptin and placebo is at 120 min post-dose (90 min post-meal) of 1.5 mmol/L. Maximum PPG and serum insulin were achieved at ˜1 h post-dose (0.5 h post-meal).

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A pharmaceutical composition, comprising:
a) a low-dose, therapeutically-effective amount of a dipeptidyl peptidase IV (DPP IV) inhibitor;
b) a low-dose, therapeutically-effective amount of a biguanide;
c) a low-dose, therapeutically-effective amount of a sulfonylurea; and
d) at least one pharmaceutically-acceptable excipient, wherein (a), (b), and (c) are each at about 20% to about 75% of a lowest diabetes therapeutic dose (LDTD).

2. The pharmaceutical composition of claim 1, wherein the DPP IV inhibitor is a gliptin.

3. The pharmaceutical composition of claim 1, wherein the DPP IV inhibitor is sitagliptin, vildagliptin, saxagliptin, linagliptin, gemigliptin, anagliptin, teneligliptin, alogliptin, trelagliptin, omarigliptin, evogliptin, gosogliptin, dutogliptin, or a pharmaceutically acceptable salt or hydrate thereof.

4. The pharmaceutical composition of claim 1, wherein the DPP IV inhibitor is sitagliptin or a pharmaceutically acceptable salt thereof.

5. The pharmaceutical composition of claim 4, wherein the DPP IV inhibitor is sitagliptin phosphate.

6. The pharmaceutical composition of claim 1, wherein the biguanide is metformin or a pharmaceutically acceptable salt or hydrate thereof.

7. The pharmaceutical composition of claim 6, wherein the biguanide is metformin hydrochloride.

8. The pharmaceutical composition of claim 6 or claim 7, wherein the metformin is formulated for immediate release.

9. The pharmaceutical composition of claim 6 or claim 7, wherein the metformin is formulated for slow release.

10. The pharmaceutical composition of claim 1, wherein the sulfonylurea is acetohexamide, carbutamide, chlorpropamide, glycyclamide (tolhexamide), metahexamide, tolazamide, tolbutamide, glibenclamide (glyburide), glibornuride, gliclazide, glipizide, gliquidone, glisoxepide, glyclopyramide, glimepiride, or a pharmaceutically acceptable salt or hydrate thereof.

11. The pharmaceutical composition of claim 10, wherein the sulfonylurea is glimepiride.

12. The pharmaceutical composition of any one of claims 1-11, wherein a dose of each (a), (b), and (c) is from about 40% to about 70% of the lowest diabetes therapeutic dose (LDTD).

13. The pharmaceutical composition of any one of claims 1-11, wherein a dose of each (a), (b), and (c) is from about 40% to about 60% of the lowest diabetes therapeutic dose (LDTD).

14. The pharmaceutical composition of any one of claims 1-11, wherein a dose of each (a), (b), and (c) is from about 45% to about 55% of the lowest diabetes therapeutic dose (LDTD).

15. The pharmaceutical composition of claim 12, wherein the DPP IV inhibitor is about 70% of the lowest diabetes therapeutic dose (LDTD) for the DPP IV inhibitor.

16. The pharmaceutical composition of claim 1, wherein the DPP IV inhibitor is sitagliptin, and a dose of sitagliptin is about 17.5 mg.

17. The pharmaceutical composition of any one of claims 1-14, wherein the DPP IV inhibitor is about 50% of the lowest diabetes therapeutic dose (LDTD) for the DPP IV inhibitor.

18. The pharmaceutical composition of claim 1, wherein the DPP IV inhibitor is sitagliptin, and a dose of sitagliptin is about 12.5 mg.

19. The pharmaceutical composition of any one of claims 1-18, wherein the biguanide is about 70% of the lowest diabetes therapeutic dose (LDTD) for the biguanide.

20. The pharmaceutical composition of claim 1, wherein the biguanide is metformin hydrochloride, and a dose of metformin hydrochloride is about 350 mg.

21. The pharmaceutical composition of any one of claims 1-18, wherein the biguanide is about 50% of the lowest diabetes therapeutic dose (LDTD) for the biguanide.

22. The pharmaceutical composition of claim 1, wherein the biguanide is metformin hydrochloride, and a dose of metformin hydrochloride is about 250 mg.

23. The pharmaceutical composition of any one of claims 1-22, wherein the sulfonylurea is about 50% of the lowest diabetes therapeutic dose (LDTD) for the sulfonylurea.

24. The pharmaceutical composition of claim 1, wherein the sulfonylurea is glimepiride, and a dose of the glimepiride is about 0.5 mg.

25. The pharmaceutical composition of claim 1, wherein the DPP W inhibitor is sitagliptin, the biguanide is metformin, and the sulfonylurea is glimepiride.

26. The pharmaceutical composition of claim 25, wherein a dose of sitagliptin is from about 5.0 mg to about 18.75 mg, a dose of metformin is from about 100 mg to about 375 mg, and a dose of glimepiride is from about 0.2 mg to about 0.75 mg.

27. The pharmaceutical composition of claim 25, wherein the dose of sitagliptin is from about 10 mg to about 16.25 mg, the dose of metformin is from about 200 mg to about 325 mg, and the dose of glimepiride is from about 0.4 mg to about 0.65 mg.

28. The pharmaceutical composition of claim 25, wherein the dose of sitagliptin is from about 10 mg to about 15 mg, the dose of metformin is from about 200 mg to about 300 mg, and the dose of glimepiride is from about 0.4 mg to about 0.6 mg.

29. The pharmaceutical composition of claim 25, wherein the dose of sitagliptin is from about 11.25 mg to about 13.75 mg, the dose of metformin is from about 225 mg to about 275 mg, and the dose of glimepiride is from about 0.45 mg to about 0.55 mg.

30. The pharmaceutical composition of claim 25, wherein the dose of sitagliptin is about 12.5 mg, the dose of metformin is about 250 mg, and the dose of glimepiride is about 0.5 mg.

31. The pharmaceutical composition of any one of claims 1-11, wherein the dose of each (a), (b), and (c) is from about 30% to about 40% of the lowest diabetes therapeutic dose (LDTD).

32. The pharmaceutical composition of any one of claims 1-11, wherein the dose of each (a), (b), and (c) is from about 30% to about 35% of the lowest diabetes therapeutic dose (LDTD).

33. The pharmaceutical composition of claim 31 or claim 32, wherein the sulfonylurea is about 33% of the lowest diabetes therapeutic dose (LDTD) for the sulfonylurea.

34. The pharmaceutical composition of claim 33, wherein the sulfonylurea is glimepiride, and the dose of sulfonylurea is about 0.33 mg.

35. The pharmaceutical composition of claim 31, wherein the DPP IV inhibitor is sitagliptin, the biguanide is metformin, and the sulfonylurea is glimepiride.

36. The pharmaceutical composition of claim 35, wherein the dose of sitagliptin is from about 7.5 mg to about 10 mg, the dose of metformin is from about 150 mg to about 200 mg, and the dose of glimepiride is from about 0.3 mg to about 0.4 mg.

37. The pharmaceutical composition of claim 35, wherein the dose of sitagliptin is about 8.25 mg, the dose of metformin is about 165 mg, and the dose of glimepiride is about 0.33 mg.

38. The pharmaceutical composition of any one of claims 1-11, wherein the dose of each (a), (b), and (c) is from about 20% to about 30% of the lowest diabetes therapeutic dose (LDTD).

39. The pharmaceutical composition of any one of claims 1-11, wherein the dose of each (a), (b), and (c) is from about 22% to about 28% of the lowest diabetes therapeutic dose (LDTD).

40. The pharmaceutical composition of claim 38 or 39, wherein the sulfonylurea is about 25% of the lowest diabetes therapeutic dose (LDTD) for the sulfonylurea.

41. The pharmaceutical composition of claim 33, wherein the sulfonylurea is glimepiride, and the dose of sulfonylurea is about 0.25 mg.

42. The pharmaceutical composition of claim 38, wherein the DPP IV inhibitor is sitagliptin, the biguanide is metformin, and the sulfonylurea is glimepiride.

43. The pharmaceutical composition of claim 42, wherein the dose of sitagliptin is from about 5 mg to about 7.5 mg, the dose of metformin is from about 100 mg to about 150 mg, and the dose of glimepiride is from about 0.2 mg to about 0.3 mg.

44. The pharmaceutical composition of claim 42, wherein the dose of sitagliptin is about 6.25 mg, the dose of metformin is about 150 mg, and the dose of glimepiride is about 0.25 mg.

45. The pharmaceutical composition of any one of claims 1-44, wherein the pharmaceutical composition is in the form of pill, tablet, or capsule.

46. The pharmaceutical composition of any one of claims 1-44, wherein the pharmaceutical composition is suitable for oral administration.

47. The pharmaceutical composition of any of claims 1-46, wherein the pharmaceutical composition does not comprise any further additional anti-hyperglycemic or anti-diabetic agents.

48. The pharmaceutical composition of any of claims 1-46, wherein the combination of a), b), and c) produces a synergistic effect.

49. The pharmaceutical composition of any of claims 1-48, wherein the pharmaceutical composition produces a larger decrease in 2 hour post-prandial glucose as compared to a maximum decrease in the post-prandial glucose obtained from about 100 mg of sitagliptin.

50. The pharmaceutical composition of any of claims 1-48, wherein the pharmaceutical composition produces a larger decrease in 2 hour post-prandial glucose as compared to a maximum decrease in the post-prandial glucose obtained from about 850 mg of metformin.

51. The pharmaceutical composition of any of claims 1-48, wherein the pharmaceutical composition produces a larger decrease in 2 hour post-prandial glucose as compared to a maximum decrease in the post-prandial glucose obtained from about 1700 mg of metformin.

52. A pharmaceutical composition, comprising:
a) a low-dose, therapeutically-effective amount of a dipeptidyl peptidase IV (DPP IV) inhibitor;
b) a low-dose, therapeutically-effective amount of a biguanide;
c) a low-dose, therapeutically-effective amount of a sulfonylurea; and
d) at least one pharmaceutically-acceptable excipient, wherein (a) and (b) are each at about 65%-75% of a lowest diabetes therapeutic dose (LDTD), and (c) is at about 45%-55% of the lowest diabetes therapeutic dose (LDTD).

53. The pharmaceutical composition of claim 52, wherein the DPP IV inhibitor is sitagliptin and a dose of sitagliptin is from about 16.25 mg to about 18.75 mg.

54. The pharmaceutical composition of claim 52, wherein the biguanide is metformin and a dose of metformin is from about 325 mg to about 375 mg.

55. The pharmaceutical composition of claim 52, wherein the sulfonylurea is glimepiride, and a dose of glimepiride from about 0.45 mg to about 0.55 mg.

56. The pharmaceutical composition of claim 52, wherein the DPP IV inhibitor is at about 70% of the lowest diabetes therapeutic dose (LDTD) for the DPP IV inhibitor.

57. The pharmaceutical composition of claim 52, wherein the biguanide is at about 70% of the lowest diabetes therapeutic dose (LDTD) for the biguanide.

58. The pharmaceutical composition of claim 52, wherein the sulfonylurea is at about 50% of the lowest diabetes therapeutic dose (LDTD) for the sulfonylurea.

59. The pharmaceutical composition of claim 52, wherein the DPP IV inhibitor is sitagliptin, the biguanide is metformin, and the sulfonylurea is glimepiride.

60. The pharmaceutical composition of claim 59, wherein the dose of sitagliptin is about 17.5 mg, the dose of metformin is about 350 mg, and the dose of glimepiride is about 0.5 mg.

61. The pharmaceutical composition of claim 52, wherein the DPP IV inhibitor is sitagliptin and the dose of the sitagliptin is about 17.5 mg.

62. The pharmaceutical composition of claim 52, wherein the biguanide is metformin and the dose of the metformin is about 350 mg.

63. The pharmaceutical composition of claim 52, wherein the sulfonylurea is glimepiride and the dose of the glimepiride is about 0.5 mg.

64. The pharmaceutical composition of any one of claims 52-63, wherein the pharmaceutical composition is suitable for oral administration.

65. The pharmaceutical composition of any one of claims 52-63, wherein the pharmaceutical composition is in the form of pill, tablet or capsule.

66. The pharmaceutical composition of any one of claims 52-65, wherein the metformin is formulated for immediate release.

67. The pharmaceutical composition of any one of claims 52-65, wherein the metformin is formulated for slow release.

68. The pharmaceutical composition of any of claims 52-67, wherein the pharmaceutical composition does not comprise any further additional anti-hyperglycemic or anti-diabetic agents.

69. The pharmaceutical composition of any of claims 52-67, wherein the combination of a), b), and c) produces a synergistic effect.

70. The pharmaceutical composition of any of claims 52-67, wherein the pharmaceutical composition produces a larger decrease in 2 hour post-prandial glucose as compared to a maximum decrease in the post-prandial glucose obtained from about 100 mg of sitagliptin.

71. The pharmaceutical composition of any of claims 52-67, wherein the pharmaceutical composition produces a larger decrease in 2 hour post-prandial glucose as compared to a maximum decrease in the post-prandial glucose obtained from about 850 mg of metformin.

72. The pharmaceutical composition of any of claims 52-67, wherein the pharmaceutical composition produces a larger decrease in 2 hour post-prandial glucose as compared to a maximum decrease in the post-prandial glucose obtained from about 1700 mg of metformin.

73. A pharmaceutical composition, comprising a combination of:
a) about 17.5 mg of sitagliptin;
b) about 350 mg of metformin
c) about 0.5 mg of glimepiride; and
d) at least one pharmaceutically-acceptable excipient.

74. The pharmaceutical composition of claim 73, wherein the combination is synergistic.

75. The pharmaceutical composition of claim 73 or 74, wherein the pharmaceutical composition is in the form of pill, tablet, or capsule.

76. The pharmaceutical composition of claim 73 or 74, wherein the pharmaceutical composition is suitable for oral administration.

77. The pharmaceutical composition of any one of claims 73-76, wherein the metformin is formulated for immediate release.

78. The pharmaceutical composition of any one of claims 73-76, wherein the metformin is formulated for slow release.

79. The pharmaceutical composition of any of claims 73-78, wherein the pharmaceutical composition does not comprise any further additional anti-hyperglycemic or anti-diabetic agents.

80. The pharmaceutical composition of any of claims 73-78, wherein the combination of a), b), and c) produces a synergistic effect.

81. The pharmaceutical composition of any of claims 73-80, wherein the pharmaceutical composition produces a larger decrease in 2 hour post-prandial glucose as compared to a maximum decrease in the post-prandial glucose obtained from about 100 mg of sitagliptin.

82. The pharmaceutical composition of any of claims 73-80, wherein the pharmaceutical composition produces a larger decrease in 2 hour post-prandial glucose as compared to a maximum decrease in the post-prandial glucose obtained from about 850 mg of metformin.

83. The pharmaceutical composition of any of claims 73-80, wherein the pharmaceutical composition produces a larger decrease in 2 hour post-prandial glucose as compared to a maximum decrease in the post-prandial glucose obtained from about 1700 mg of metformin.

84. A synergistic, ultra-low dose, anti-diabetic drug combination, consisting of:
a) about 16.25 mg to about 18.75 mg of sitagliptin, or a salt or hydrate thereof;
b) about 325 mg to about 375 mg of metformin, or a salt or hydrate thereof;
c) about 0.45 mg to about 0.55 mg of glimepiride, or a salt or hydrate thereof; and
d) at least one excipient.

85. The combination of claim 84, wherein the combination does not comprise any further additional anti-hyperglycemic or anti-diabetic agents.

86. The combination of claim 84, wherein the combination of a), b), and c) produces a synergistic effect.

87. The combination of claim 84, wherein the combination produces a larger decrease in 2 hour post-prandial glucose as compared to a maximum decrease in the post-prandial glucose obtained from about 100 mg of sitagliptin, from about 850 mg of metformin, or from about 1700 mg of metformin.

88. A method of treating diabetes in a subject in need thereof comprising administering the pharmaceutical composition of any one of claims 1-83.

89. The method of claim 88, wherein the subject has persisting elevation of blood sugar after treatment with one or two of a DPP IV inhibitor, a biguanide, or a sulfonylurea at the LDTD or higher dose.

90. The method of claim 88, wherein the administration of the pharmaceutical composition is an initial or first-line treatment of diabetes.

91. A method of improving, slowing the progression of, or delaying a metabolic disorder, wherein the metabolic disorder comprises diabetes mellitus, impaired glucose tolerance, impaired fasting blood glucose, hyperglycemia, postprandial hyperglycemia, overweight, obesity, metabolic syndrome, impaired renal function, gestational diabetes, new onset diabetes after transplantation (NODAT) and complications associated therewith, or post-transplant metabolic syndrome (PTMS) and complications associated therewith, comprising administering to a subject in need thereof the pharmaceutical composition of any one of claims 1-83.

92. A method of treating diabetes in a subject in need thereof comprising administering a synergistic, ultra-low dose, anti-diabetic drug combination, consisting of:
a) about 16.25 mg to about 18.75 mg of sitagliptin, or a salt or hydrate thereof;
b) about 325 mg to about 375 mg of metformin, or a salt or hydrate thereof;
c) about 0.45 mg to about 0.55 mg of glimepiride, or a salt or hydrate thereof; and
d) at least one excipient.

93. The method of claim 92, wherein the combination does not comprise any further additional anti-hyperglycemic or anti-diabetic agents.

94. The method of claim 92, wherein the combination of a), b), and c) produces a synergistic effect.

95. The method of claim 92, wherein the combination produces a larger decrease in 2 hour post-prandial glucose as compared to a maximum decrease in the post-prandial glucose obtained from about 100 mg of sitagliptin, from about 850 mg of metformin, or from about 1700 mg of metformin.
